(19) United States
(12) Patent Application Publication
(10) Pub. No.: US 2022/0177487 A1
(43) Pub. Date: Jun. 9, 2022

(54) FUSED POLYCYCLIC PYRIDONE COMPOUNDS AS INFLUENZA VIRUS REPLICATION INHIBITORS

(71) Applicant: NIKANG THERAPEUTICS, INC., Wilmington, DE (US)

(72) Inventors: Jiping FU, Danville, CA (US); Yigang HE, Newark, DE (US); Yan LOU, Pleasanton, CA (US)

(21) Appl. No.: 17/600,587

(22) PCT Filed: Mar. 31, 2020
(86) PCT No.: PCT/US2020/025908

§ 371 Date: Sep. 30, 2021

Related U.S. Application Data
(60) Provisional application No. 62/827,754, filed on Apr. 1, 2019.

(51) Int. Cl. C07D 491/22; C07D 471/22; A61K 45/06; A61P 31/16

(52) U.S. Cl. CPC C07D 491/22; C07D 471/22; A61K 9/0053; A61P 31/16; A61K 45/06

(57) ABSTRACT

Provided are compounds of Formula (IA) that inhibit orthomyxovirus replication such as Influenza viruses and are accordingly useful for treatment of viral infections caused by orthomyxoviruses. Also provided are pharmaceutical compositions containing these compounds and methods of using these compounds to treat or prevent viral infections caused by orthomyxovirus such as Influenza viruses.

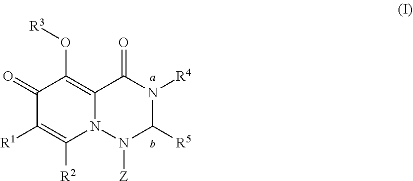

### CROSS-REFERENCE TO RELATED APPLICATIONS

This application claims the benefit of U.S. Provisional Application Ser. No. 62/827,754, filed on Apr. 1, 2019, the entire contents of which are herein incorporated by reference in its entirety.

### FIELD OF THE DISCLOSURE

The invention provides compounds that inhibit orthomyxovirus replication and are accordingly useful for the treatment of viral infections caused by orthomyxoviruses such as Influenza viruses. The invention further provides pharmaceutical compositions containing these compounds and methods of using these compounds to treat viral infections caused by orthomyxovirus such as Influenza viruses.

### BACKGROUND

Orthomyxoviruses have negative-sense single stranded RNA genomes, and replicate in the nucleus of infected cells, as they lack the machinery to generate the cap structure to produce their own mRNA. Members of the orthomyxovirus family have an RNA-dependent RNA polymerase with endonuclease activity that cleaves a section of the capped 5′-end of cellular mRNA and then uses this capped fragment as a primer for synthesis of viral mRNA by transcribing the rest of the viral RNA genome. This process is known as cap-snatching. This is due to the need of mRNA to have a 5′ cap in order to be recognized by the cell's ribosome for translation. The above viral endonuclease has been recognized as a promising target for development of antivirals effective against orthomyxoviruses. ACS Med. Chun. Letters. 2014, vol. 5, 61-64.

The orthomyxovirus family includes influenza A, influenza B and influenza C viruses, all of which can infect humans, as well as several other genera of viruses that generally do not infect humans. Influenza A and B are the most virulent of these pathogens in humans, often accounting for the majority of serious cases of influenza during a typical flu season. It is estimated that influenza kills as many as 40,000 people per year in the U.S., in spite of the widespread use of vaccines to reduce the incidence of influenza. Therefore, there is a need for antiviral therapeutics effective to treat influenza such as influenza A and B viruses.

### SUMMARY

Disclosed herein are compounds that inhibit replication of orthomyxoviruses, including at least one of influenza A, influenza B and influenza C. Without being bound by theory, it is believed these compounds achieve their antiviral effects by inhibiting the endonuclease function of the viral RNA polymerase.

In a first aspect provided is a compound of Formula (IA):

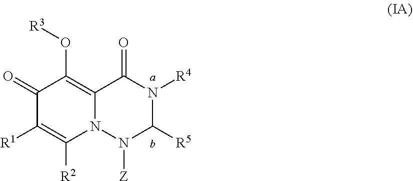

wherein:
R¹ is hydrogen, halo, alkyl, cyano, carboxy, alkoxycarbonyl, aminocarbonyl, alkylaminocarbonyl, dialkylaminocarbonyl, hydroxy, haloalkoxy, or alkyl substituted with one or two substituents independently selected from halo, hydroxy, alkoxy, amino, alkylamino, and dialkylamino;
R² is hydrogen, halo, alkyl, haloalkoxy, hydroxy, or alkyl substituted with one, two, or three substituents independently selected from halo, CN, hydroxy, alkoxy, amino, alkylamino, and dialkylamino;
R³ is hydrogen, —C(O)R⁶, —C(O)—O—R⁷, —C(R⁸R⁹)—O—C(O)R¹⁰, —C(R¹¹R¹²)—O—C(O)—OR¹³,
   —P(═O)(OR¹⁴)(OR¹⁵), —(CR¹⁶R¹⁷)—O—P(═O)(OR¹⁸)(OR¹⁹), —C(O)—N(R²⁰R²¹), or —C(R²²R²³)—O—C(O)N(R²⁴R²⁵) where R⁶, R⁷, R¹⁰, R¹³, R¹⁴, R¹⁵, R¹⁸, R¹⁹, R²⁰, R²¹, R²⁴ and R²⁵ are independently hydrogen, alkyl, phenyl, pyridyl, cycloalkyl, and a 3-6 membered heterocyclic ring wherein alkyl, phenyl, pyridyl, cycloalkyl, and 3-6 membered heterocyclic ring are independently optionally substituted with one or two substituents independently selected from halo, cyano, hydroxy, amino, alkyl, carboxy, alkoxycarbonyl, phenyl, alkoxy, haloalkyl, and haloalkoxy;
   and R⁸, R⁹, R¹¹, R¹², R¹⁶, R¹⁷, R²², and R²³ are independently hydrogen or alkyl;

R⁴ and R⁵ together with the atoms to which they are attached form a ring of formula (a), (b), (c), or (d):

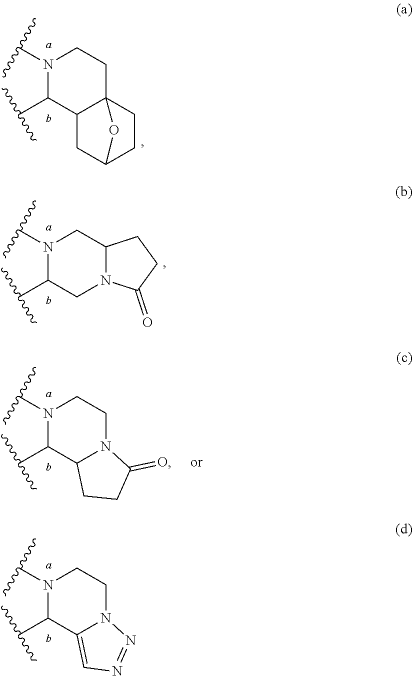 [SMILES: *C1C2CC3CCC2(CCN1*)O3 ; *C1CN2C(=O)CCC2CN1* ; *C1C2CCC(=O)N2CCN1* ; *C1c2cnnn2CCN1*]

wherein ring of formula (a), (b), (c), or (d) can optionally be substituted with one or two substituents independently selected from alkyl, alkoxy, hydroxy, halo, haloalkyl, haloalkoxy, and cyano; and

Z is:

(i) a ring of formula (i):

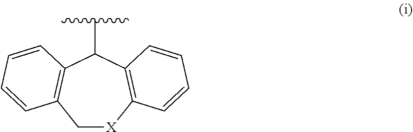

where:

wherein X is CH₂, S, S(O), S(O)₂, or O; and ring of formula (i) is substituted with one, two, three, or four substituents independently selected from hydrogen, halo, alkyl, haloalkyl, alkoxy, haloalkoxy, alkynyl, and cyano; or

(ii) a ring of formula (ii):

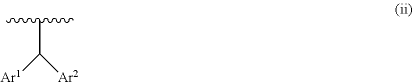

wherein Ar¹ and Ar² are independently selected from phenyl and a 5-6 membered heteroaryl ring containing 1-3 heteroatoms independently selected from N, O and S wherein each of the Ar¹ and Ar² is independently optionally substituted with one, two, or three substituents independently selected from halo, alkyl, haloalkyl, alkoxy, haloalkoxy, alkynyl, and cyano; or

a pharmaceutically acceptable salt thereof.

In an embodiment of the first aspect, provided is a compound of Formula (I):

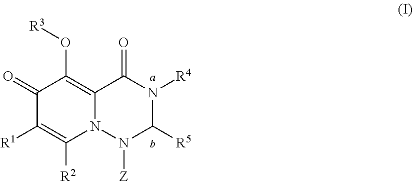

wherein:

R¹ is hydrogen, halo, alkyl, cyano, carboxy, alkoxycarbonyl, aminocarbonyl, alkylaminocarbonyl, dialkylaminocarbonyl, or alkyl substituted with one or two substituents independently selected from halo, hydroxy, alkoxy, amino, alkylamino, and dialkylamino;

R² is hydrogen, halo, alkyl, haloalkoxy, or alkyl substituted with one, two, or three substituents independently selected from halo, CN, hydroxy, alkoxy, amino, alkylamino, and dialkylamino;

and R³, R⁴, R⁵, and Z are as defined in the first aspect above.

In a second aspect, provided is a pharmaceutical composition comprising a compound of the Formula (I) (or any of the embodiments thereof described herein), or a pharmaceutically acceptable salt thereof; and a pharmaceutically acceptable excipient.

In a third aspect, provided is a method of treating a disease, caused by a virus having cap-dependent endonuclease, in a patient, comprising administering to the patient in need thereof a compound of Formula (I) (or any one of embodiments thereof disclosed herein).

In a fourth aspect, provided is a method of treating a disease, caused by Influenza A, Influenza B, and/or Influenza C viruses, in a patient, comprising administering to the patient in need thereof a compound of Formula (I) (or any one of embodiments thereof disclosed herein). In a first sub-embodiment of the fourth aspect, the virus is an Influenza A. In a second sub-embodiment of the fourth aspect, the virus is Influenza B. In a third sub-embodiment of the fourth aspect, the virus is Influenza C. In a fourth sub-embodiment of the fourth aspect, the disease is caused by Influenza A and Influenza B. In a fifth sub-embodiment of the fourth aspect, the disease is caused by Influenza A and Influenza C. In a sixth sub-embodiment of the fourth aspect, the disease is caused by Influenza B and Influenza C.

In a fifth aspect, the disclosure is directed to a compound of Formula (I), (or any embodiments thereof described herein) or a pharmaceutically acceptable salt thereof for use as a medicament or for use in therapy.

In a sixth aspect, provided is the use of a compound of Formula (I) or a pharmaceutically acceptable salt thereof (and any embodiments thereof disclosed herein) in the manufacture of a medicament for treating a disease in a patient caused by an Influenza virus. In a first sub-embodiment of the sixth aspect, the virus is Influenza A, Influenza B, and/or Influenza C viruses. In a second sub-embodiment of the sixth aspect, the virus is an Influenza A. In a third sub-embodiment of the sixth aspect, the virus is Influenza B. In a fourth sub-embodiment of the sixth aspect, the virus is Influenza C. In a fifth sub-embodiment of the sixth aspect, the disease is caused by Influenza A and Influenza B. In a sixth sub-embodiment of the sixth aspect, the disease is caused by Influenza A and Influenza C. In a seventh sub-embodiment of the sixth aspect, the disease is caused by Influenza B and Influenza C.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkynyl” means a linear saturated monovalent hydrocarbon radical of two to six carbon atoms or a branched saturated monovalent hydrocarbon radical of three to six carbon atoms containing a triple bond, e.g., ethynyl, propynyl, and the like.

“Amino” means a —NH₂.

“Aminocarbonyl” means a —CONH₂.

“Alkylamino” means a —NHR radical where R is alkyl as defined above, e.g., methylamino, ethylamino, propylamino, or 2-propylamino, and the like.

“Alkylaminocarbonyl” means a —CONHR radical where R is alkyl as defined above, e.g., methylaminocarbonyl, ethylaminocarbonyl, or 2-propylaminocarbonyl, and the like.

“Alkoxy” means a —OR radical where R is alkyl as defined above, e.g., methoxy, ethoxy, propoxy, or 2-propoxy, n-, iso-, or tert-butoxy, and the like.

“Alkoxycarbonyl” means a —C(O)OR radical where R is alkyl as defined above, e.g., methoxycarbonyl, ethoxycarbonyl, and the like.

“Cycloalkyl” means a monocyclic saturated monovalent hydrocarbon radical of three to ten carbon atoms. Examples include, but are not limited to, cyclopropyl, cyclobutyl, cyclopentyl, or cyclohexyl, and the like.

“Carboxy” means —COOH.

“Cyano” means —CN.

“Dialkylamino” means a —NRR′ radical where R and R′ are independently alkyl as defined above, e.g., dimethylamino, methylethylamino, and the like.

“Dialkylminocarbonyl” means a —CONRR′ radical where R and R′ are alkyl as defined above, e.g., dimethylaminocarbonyl, diethylaminocarbonyl, or (methyl)2-propylaminocarbonyl, and the like.

“Halo” means fluoro, chloro, bromo, or iodo, preferably fluoro or chloro.

“Haloalkyl” means alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means an —OR radical where R is haloalkyl as defined above e.g., —OCF₃,

—OCHF₂, and the like. When R is haloalkyl where the alkyl is substituted with only fluoro, it is referred to in this Application as fluoroalkoxy.

“Heterocyclic” means a saturated or unsaturated monovalent monocyclic ring of 3 to 6 ring atoms in which one, two, or three ring atoms are heteroatom selected from N, O, and S(O)ₙ, where n is an integer from 0 to 2, the remaining ring atoms being carbon, unless otherwise stated. Additionally, one or two ring carbon atoms in the heterocyclyl ring can optionally be replaced by a —C(O)— group. More specifically the term heterocyclyl includes, but is not limited to, pyrrolidinyl, piperidinyl, homopiperidinyl, 2-oxopyrrolidinyl, 2-oxopiperidinyl, morpholinyl, piperazinyl, tetrahydropyranyl, thiomorpholinyl, and the like. When the heterocyclyl ring is unsaturated, it can contain one or two ring double bonds provided that the ring is not aromatic. When the heterocyclyl group contains at least one nitrogen atom, it is also referred to herein as heterocycloamino and is a subset of the heterocyclyl group.

“Heteroaryl” means a monovalent monocyclic or bicyclic aromatic radical of 5 to 10 ring atoms, where one or more, (in one embodiment, one, two, or three), ring atoms are heteroatom selected from N, O, or S, the remaining ring atoms being carbon, unless otherwise stated. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms it is also referred to herein as 5- or 6-membered heteroaryl.

The term “oxo,” as used herein, alone or in combination, refers to ═(O).

When needed, any definition herein may be used in combination with any other definition to describe a composite structural group. By convention, the trailing element of any such definition is that which attaches to the parent moiety. For example, the composite group alkoxyalkyl means that an alkoxy group attached to the parent molecule through an alkyl group.

The present disclosure also includes protected derivatives of compounds of the present disclosure (I). For example, when compounds of the present disclosure contain groups such as hydroxy, carboxy, or any group containing a nitrogen atom(s), these groups can be protected with suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes polymorphic forms of the compound of Formula (I) and/or a pharmaceutically acceptable salt thereof.

The present disclosure also includes prodrugs of the compound of Formula (I) and/or a pharmaceutically acceptable salt thereof. The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Prodrugs are often useful because, in some situations, they may be easier to administer than the compound, or parent drug. They may, for instance, be bioavailable by oral administration whereas the parent drug is not. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference in its entirety.

The compounds of Formula (I) may have asymmetric centers. Compounds of Formula (I) containing an asymmetrically substituted atom may be isolated in optically active or racemic forms. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. All chiral, diastereomeric, all mixtures of chiral or diastereomeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding (S) stereoisomer as an impurity and vice versa.

Certain compounds of Formula (I) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all hydrates of a compound of Formula (I) are within the scope of this disclosure.

The compounds of Formula (I) may also contain unnatural amounts of isotopes at one or more of the atoms that constitute such compounds. Unnatural amounts of an isotope may be defined as ranging from the amount found in nature to an amount 100% of the atom in question, that differ only in the presence of one or more isotopically enriched atoms. Exemplary isotopes that can be incorporated into compounds of Formula (I) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds Formula (I), including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Optional” or “optionally” means that the subsequently described event or circumstance may but need not occur, and that the description includes instances where the event or circumstance occurs and instances in which it does not. For example, “phenyl group optionally substituted with an alkyl group” means that the alkyl may but need not be present, and the description includes situations where the phenyl group is substituted with an alkyl group and situations where the phenyl group is not substituted with alkyl.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

In one embodiment, treating means preventing the disease. In another embodiment, treating means inhibiting or relieving the disease.

A “therapeutically effective amount” means the amount of a compound of Formula (I) and/or a pharmaceutically acceptable salt thereof that, when administered to a patient for treating a disease, is sufficient to affect such treatment for the disease. The “therapeutically effective amount” will vary depending on the compound, the disease and its severity and the age, weight, etc., of the mammal to be treated.

The terms “inhibiting” and “reducing,” or any variation of these terms in relation of endonuclease, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of endonuclease activity of viral RNA polymerase compared to its normal activity.

Representative compounds of Formula (I) are disclosed in Table (I) below

TABLE 1

| Cpd # | Structure | Name |
|---|---|---|
| 1 | 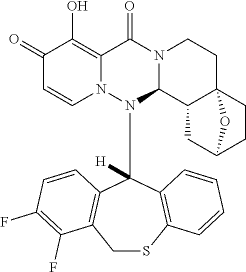 [SMILES: O=C1c2c(O)c(=O)ccn2N([C@@H]2c3ccccc3SCc3c2ccc(F)c3F)[C@@H]2[C@H]3C[C@@H]4CC[C@@]3(CCN12)O4] | (2S,4aS,14aR,14bR)-14-((S)-7,8-difluoro-6,11- dihydro-dibenzo[b,e]thiepin-11-yl)-9-hydroxy- 1,3,4,5,6,14,14a,14b-octahydro-2H-2,4a- epoxypyrido[1′,2′:1,6][1,2,4]tiazino[3,4-a]- isoquinoline-8,10-dione |
| 2 | 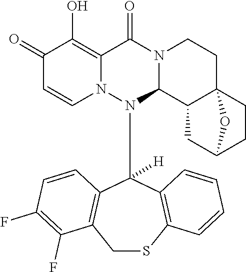 [SMILES: O=C1c2c(O)c(=O)ccn2N([C@H]2c3ccccc3SCc3c2ccc(F)c3F)[C@@H]2[C@H]3C[C@@H]4CC[C@@]3(CCN12)O4] | (2S,4aS,14aR,14bR)-14-((R)-7,8-difluoro-6,11- dihydro-dibenzo[b,e]thiepin-11-yl)-9-hydroxy- 1,3,4,5,6,14,14a,14b-octahydro-2H-2,4a- epoxypyrido[1′,2′:1,6][1,2,4]triazino[3,4-a]- isoquinoline-8,10-dione |
| 3 | 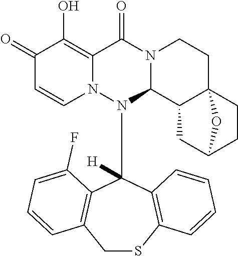 [SMILES: O=C1c2c(O)c(=O)ccn2N([C@@H]2c3ccccc3SCc3cccc(F)c32)[C@@H]2[C@H]3C[C@@H]4CC[C@@]3(CCN12)O4] | (2S,4aS,14aR,14bR)-14-((S)-10-fluoro-6,11- dihydro-dibenzo[b,e]thiepin-11-yl)-9-hydroxy- 1,3,4,5,6,14,14a,14b-octahydro-2H-2,4a- epoxypyrido[1′,2′:1,6][1,2,4]triazino[3,4-a]- isoquinoline-8,10-dione |
| 4 | 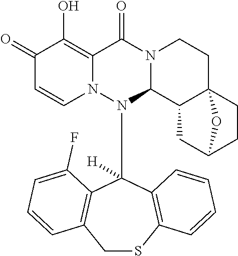 [SMILES: O=C1c2c(O)c(=O)ccn2N([C@H]2c3ccccc3SCc3cccc(F)c32)[C@@H]2[C@H]3C[C@@H]4CC[C@@]3(CCN12)O4] | (2S,4aS,14aR,14bR)-14-((R)-10-fluoro-6,11- dihydro-dibenzo[b,e]thiepin-11-yl)-9-hydroxy- 1,3,4,5,6,14,14a,14b-octahydro-2H-2,4a- epoxypyrido[1′,2′:1,6][1,2,4]triazino[3,4-a]- isoquinoline-8,10-dione |
| 5 | 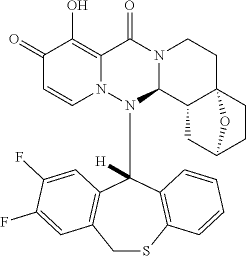 [SMILES: O=C1c2c(O)c(=O)ccn2N([C@H]2c3cc(F)c(F)cc3CSc3ccccc32)[C@@H]2[C@H]3C[C@@H]4CC[C@@]3(CCN12)O4] | (2S,4aS,14aR,14bR)-14-((S)-8,9-difluoro-6,11- dihydro-dibenzo[b,e]thiepin-11-yl)-9-hydroxy- 1,3,4,5,6,14,14a,14b-octahydro-2H-2,4a- epoxypyrido[1′,2′:1,6][1,2,4]triazino[3,4-a]- isoquinoline-8,10-dione |
| 6 | 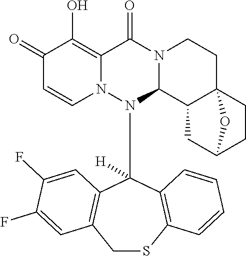 [SMILES: O=C1c2c(O)c(=O)ccn2N([C@@H]2c3cc(F)c(F)cc3CSc3ccccc32)[C@@H]2[C@H]3C[C@@H]4CC[C@@]3(CCN12)O4] | (2S,4aS,14aR,14bR)-14-((R)-8,9-difluoro-6,11- dihydro-dibenzo[b,e]thiepin-11-yl)-9-hydroxy- 1,3,4,5,6,14,14a,14b-octahydro-2H-2,4a- epoxypyrido[1′,2′:1,6][1,2,4]triazino[3,4-a]- isoquinoline-8,10-dione |
| 7 | 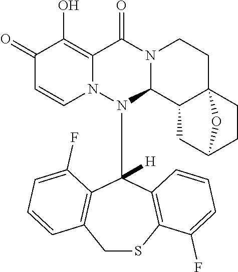 [SMILES: O=C1c2c(O)c(=O)ccn2N([C@@H]2c3cccc(F)c3SCc3cccc(F)c32)[C@@H]2[C@H]3C[C@@H]4CC[C@@]3(CCN12)O4] | (2S,4aS,14aR,14bR)-14-((S)-4,10-difluoro-6,11- dihydro-dibenzo[b,e]thiepin-11-yl)-9-hydroxy- 1,3,4,5,6,14,14a,14b-octahydro-2H-2,4a- epoxypyrido[1′,2′:1,6][1,2,4]triazino[3,4-a]- isoquinoline-8,10-dione |
| 8 | 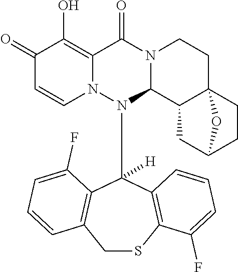 [SMILES: O=C1c2c(O)c(=O)ccn2N([C@H]2c3cccc(F)c3SCc3cccc(F)c32)[C@@H]2[C@H]3C[C@@H]4CC[C@@]3(CCN12)O4] | (2S,4aS,14aR,14bR)-14-((R)-4,10-difluoro- 6,11-dihydro-dibenzo[b,e]thiepin-11-yl)-9-hydroxy- 1,3,4,5,6,14,14a,14b-octahydro-2H-2,4a- epoxypyrido[1′,2′:1,6][1,2,4]triazino[3,4-a]- isoquinoline-8,10-dione |
| 9 | 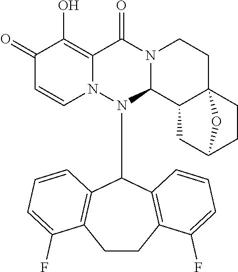 [SMILES: O=C1c2c(O)c(=O)ccn2N(C2c3cccc(F)c3CCc3c(F)cccc32)[C@@H]2[C@H]3C[C@@H]4CC[C@@]3(CCN12)O4] | (2S,4aS,14aR,14bR)-14-(1,9-difluoro-10,11- dihydro-5H-dibenzo[a,d][7]annulen-5-yl)-9-hydroxy- 1,3,4,5,6,14,14a,14b-octahydro-2H-2,4a- epoxypyrido[1′,2′:1,6][1,2,4]triazino[3,4-a]- isoquinoline-8,10-dione |
| 10 | 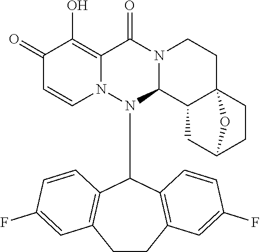 [SMILES: O=C1c2c(O)c(=O)ccn2N(C2c3ccc(F)cc3CCc3cc(F)ccc32)[C@@H]2[C@H]3C[C@@H]4CC[C@@]3(CCN12)O4] | (2S,4aS,14aR,14bR)-14-(2,8-difluoro-10,11- dihydro-5H-dibenzo[a,d][7]annulen-5-yl)-9- hydroxy-1,3,4,5,6,14,14a,14b-octahydro-2H- 2,4a-epoxypyrido[1′,2′:1,6][1,2,4]triazino[3,4- a]-isoquinoline-8,10-dione |
| 11 | 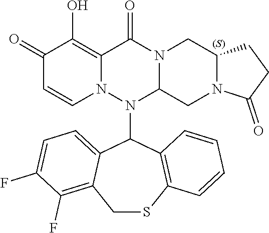 | (14aS)-6-(7,8-difluoro-6,11- dihydrobenzo[b,e]thiepin-11-yl)-11-hydroxy- 5a,6,14,14a-tetrahydro-1H,5H-pyrido[2,1-f]- pyrrolo[1′,2′:4,5]pyrazino[2,1-c][1,2,4]triazine- 3,10,12(2H)-trione |
| 12 | 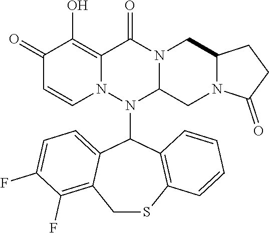 | (14aR)-6-(7,8-difluoro-6,11- dihydrobenzo[b,e]thiepin-11-yl)-11-hydroxy- 5a,6,14,14a-tetrahydro-1H,5H-pyrido[2,1-f]- pyrrolo[1′,2′:4,5]pyrazino[2,1-c][1,2,4]triazine- 3,10,12(2H)-trione |
| 13a | 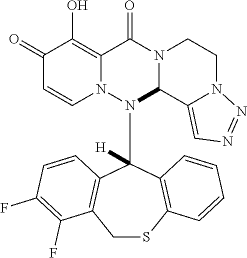 | rac-(R)-14-((S)-7,8-difluoro-6,11- dihydrodibenzo[b,e]thiepin-11-yl)-9-hydroxy- 5,6,14,14a-tetrahydro-[1,2,3]triazolo- [5′,1′:3,4]pyrazino[2,1-c]pyrido[2,1- f][1,2,4]triazine-8,10-dione |
| 13b | 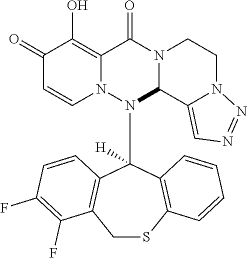 | rac-(R)-14-((R)-7,8-difluoro-6,11- dihydrodibenzo[b,e]-thiepin-11-yl)-9-hydroxy- 5,6,14,14a-tetrahydro-[1,2,3]-triazolo- [5′,1′:3,4]pyrazino[2,1-c]pyrido[2,1- f][1,2,4]triazine-8,10-dione |
| 14 | 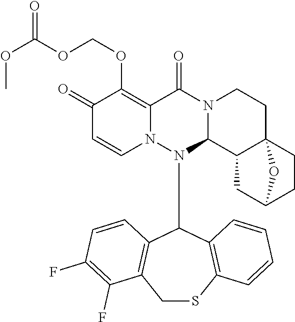 [SMILES: COC(=O)OCOc1c2n(ccc1=O)N(C1c3ccccc3SCc3c1ccc(F)c3F)[C@@H]1[C@H]3C[C@@H]4CC[C@@]3(CCN1C2=O)O4] | (((2S,4aS,14aR,14bR)-14-(7,8-difluoro-6,11- dihydro-dibenzo[b,e]thiein-11-yl)-8,10-dioxo- 1,3,4,5,6,8,10,14,14a,14b-decahydro-2H-2,4a- epoxypyrido[1′,2′:1,6][1,2,4]tiazino[3,4- a]isoquinolin-9-yl)-oxy)methyl methyl carbonate |
| 15 | 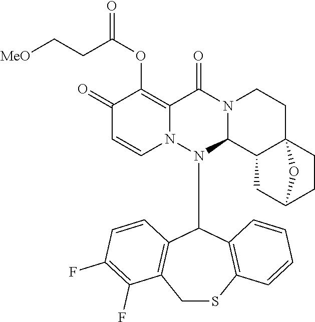 [SMILES: COCCC(=O)Oc1c2n(ccc1=O)N(C1c3ccccc3SCc3c1ccc(F)c3F)[C@@H]1[C@H]3C[C@@H]4CC[C@@]3(CCN1C2=O)O4] | (2S,4aS,14aR,14bR)-14-(7,8-difluoro-6,11- dihydro-dibenzo[b,e]thiepin-11-yl)-8,10-dioxo- 1,3,4,5,6,8,10,14,14a,14b-decahydro-2H-2,4a- epoxypyrido-[1′,2′:1,6][1,2,4]triazino[3,4- a]isoquinolin-9-yl 3-methoxypropanoate |
| 16 | 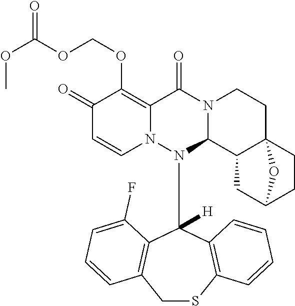 [SMILES: COC(=O)OCOc1c2n(ccc1=O)N([C@@H]1c3ccccc3SCc3cccc(F)c31)[C@@H]1[C@H]3C[C@@H]4CC[C@@]3(CCN1C2=O)O4] | (((2S,4aS,14aR,14bR)-14-((S)-10-fluoro-6,11- dihydro-dibenzo[b,e]thiepin-11-yl)-8,10-dioxo- 1,3,4,5,6,8,10,14,14a,14b-decahydro-2H-2,4a- epoxypyrido-[1′,2′:1,6][1,2,4]triazino[3,4- a]isoquinolin-9-yl)oxy)methyl methyl carbonate |
| 17 | 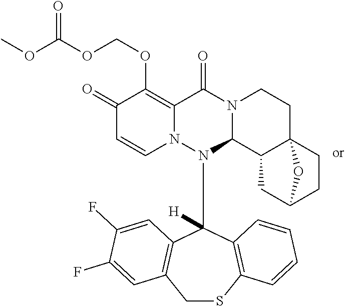 [SMILES: COC(=O)OCOc1c2n(ccc1=O)N([C@H]1c3cc(F)c(F)cc3CSc3ccccc31)[C@@H]1[C@H]3C[C@@H]4CC[C@@]3(CCN1C2=O)O4] | (((2S,4aS,14aR,14bR)-14-((S)-8,9-difluoro- 6,11-dihydrodibenzo-[b,e]thiepin-11-yl)-8,10- dioxo-1,3,4,5,6,8,10,14,14a14b,- decahydro-2H-2,4a-epoxypyrido- [1′,2′:1,6][1,2,4]triazino[3,4- a]isoquinolin-9-yl)oxy)methyl methyl carbonate or |
| | 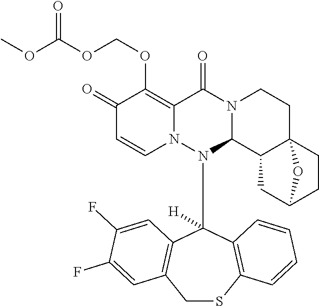 [SMILES: COC(=O)OCOc1c2n(ccc1=O)N([C@@H]1c3cc(F)c(F)cc3CSc3ccccc31)[C@@H]1[C@H]3C[C@@H]4CC[C@@]3(CCN1C2=O)O4] | (((2S,4aS,14aR,14bR)-14-((R)-8,9-difluoro- 6,11-dihydrobenzo-[b,e]thiepin-11-yl)-8,10-dioxo- 1,3,4,5,6,8,10,14,14a,14b-decahydro-2H-2,4a- epoxypyrido-[1′,2′:1,6][1,2,4]triazino[3,4- a]isoquinolin-9-yl)oxy)methyl methyl carbonate |

Additional compound of Formula (I) contemplated are provided in Table 2 below

TABLE 2

| Cpd # | Structure | Name |
|---|---|---|
| 2-1 | 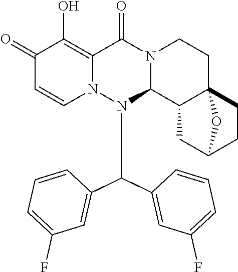 | (2S,4aS,14aR,14bR)-14-(bis(3- fluorophenyl)methyl)-9-hydroxy- 1,3,4,5,6,14,14a,14b-octahydro-2H-2,4a- epoxy-pyrido[1′,2′:1,6][1,2,4]triazino[3,4- a]isoquinoline-8,10-dione |
| 2-2 | 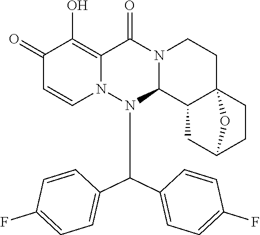 [SMILES: O=C1c2c(O)c(=O)ccn2N(C(c2ccc(F)cc2)c2ccc(F)cc2)[C@@H]2[C@H]3C[C@@H]4CC[C@@]3(CCN12)O4] | (2S,4aS,14aR,14bR)-14-(bis(4- fluorophenyl)methyl)-9-hydroxy- 1,3,4,5,6,14,14a,14b-octahydro-2H-2,4a- epoxy-pyrido[1,′,2′:1,6][1,2,4]triazino[3,4- a]isoquinoline-8,10-dione |
| 2-3 | 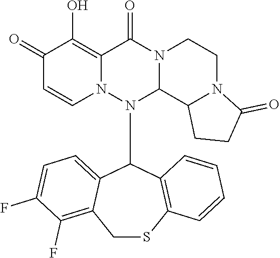 [SMILES: O=C1CCC2C3N(CCN12)C(=O)c1c(O)c(=O)ccn1N3C1c2ccccc2SCc2c1ccc(F)c2F] | 14-(7,8-difluoro-6,11- dihydrodibenzo[b,e]thiepin-11-yl)-9- hydroxy-1,5,6,14,14a,14b- hexahydropyrido[2,1-f]- pyrrolo[2′,1′:3,4]pyrazino[2,1- c][1,2,4]triazine-3,8,10(2H)-trione |
| 2-4 | 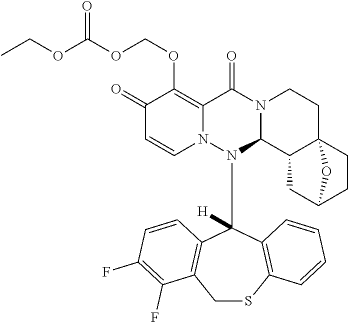 [SMILES: CCOC(=O)OCOc1c2n(ccc1=O)N([C@@H]1c3ccccc3SCc3c1ccc(F)c3F)[C@@H]1[C@H]3C[C@@H]4CC[C@@]3(CCN1C2=O)O4] | (((2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a- epoxypyrido[1′,2′:1,6][1,2,4]triazino[3,4- a]isoquinolin-9-yl)-oxy)methyl ethyl carbonate |
| 2-5 | 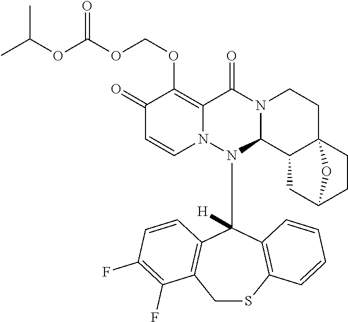 [SMILES: CC(C)OC(=O)OCOc1c2n(ccc1=O)N([C@@H]1c3ccccc3SCc3c1ccc(F)c3F)[C@@H]1[C@H]3C[C@@H]4CC[C@@]3(CCN1C2=O)O4] | (((2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)oxy)methyl isopropyl carbonate |
| 2-6 | 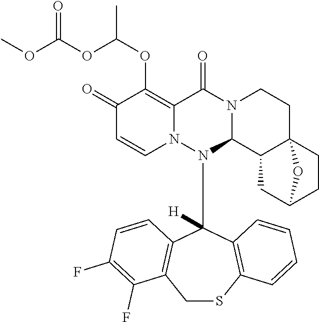 [SMILES: COC(=O)OC(C)Oc1c2n(ccc1=O)N([C@@H]1c3ccccc3SCc3c1ccc(F)c3F)[C@@H]1[C@H]3C[C@@H]4CC[C@@]3(CCN1C2=O)O4] | 1-(((2S,4aS,14aR,14bR)-14-((S)-7,8- difluoro-6,11-dihydro-dibenzo[b,e]thiepin- 11-yl)-8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)oxy)ethyl methyl carbonate |
| 2-7 | 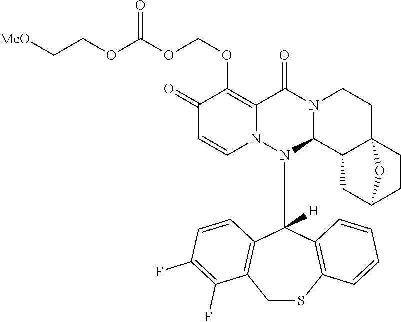 [SMILES: COCCOC(=O)OCOc1c2n(ccc1=O)N([C@@H]1c3ccccc3SCc3c1ccc(F)c3F)[C@@H]1[C@H]3C[C@@H]4CC[C@@]3(CCN1C2=O)O4] | (((2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)oxy)methyl (2-methoxyethyl) carbonate |
| 2-8 | 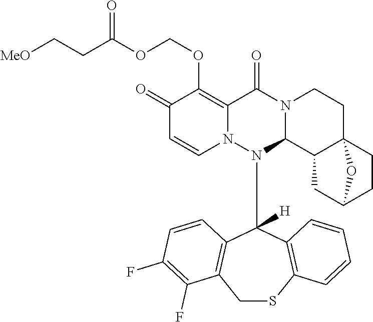 [SMILES: COCCC(=O)OCOc1c2n(ccc1=O)N([C@@H]1c3ccccc3SCc3c1ccc(F)c3F)[C@@H]1[C@H]3C[C@@H]4CC[C@@]3(CCN1C2=O)O4] | (((2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)-oxy)methyl 3-methoxypropanoate |
| 2-9 | 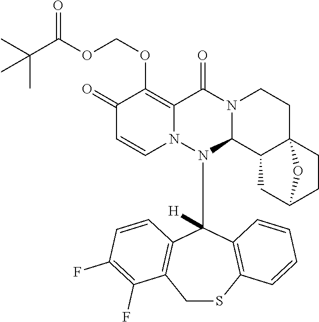 [SMILES: CC(C)(C)C(=O)OCOc1c2n(ccc1=O)N([C@@H]1c3ccccc3SCc3c1ccc(F)c3F)[C@@H]1[C@H]3C[C@@H]4CC[C@@]3(CCN1C2=O)O4] | (((2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)oxy)methyl pivalate |
| 2-10 | 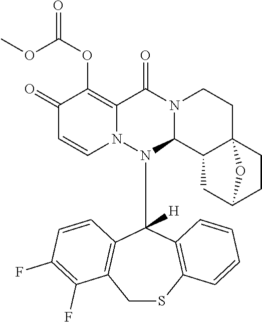 [SMILES: COC(=O)Oc1c2n(ccc1=O)N([C@@H]1c3ccccc3SCc3c1ccc(F)c3F)[C@@H]1[C@H]3C[C@@H]4CC[C@@]3(CCN1C2=O)O4] | (2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl methyl carbonate |
| 2-11 | 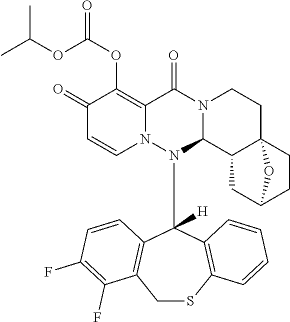 [SMILES: CC(C)OC(=O)Oc1c2n(ccc1=O)N([C@@H]1c3ccccc3SCc3c1ccc(F)c3F)[C@@H]1[C@H]3C[C@@H]4CC[C@@]3(CCN1C2=O)O4] | (2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl isopropyl carbonate |
| 2-12 | 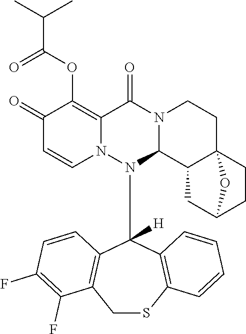 [SMILES: CC(C)C(=O)Oc1c2n(ccc1=O)N([C@@H]1c3ccccc3SCc3c1ccc(F)c3F)[C@@H]1[C@H]3C[C@@H]4CC[C@@]3(CCN1C2=O)O4] | (2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl isobutyrate |
| 2-13 | 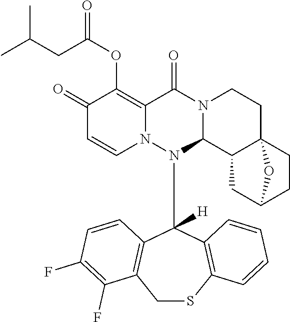 [SMILES: CC(C)CC(=O)Oc1c2n(ccc1=O)N([C@@H]1c3ccccc3SCc3c1ccc(F)c3F)[C@@H]1[C@H]3C[C@@H]4CC[C@@]3(CCN1C2=O)O4] | (2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl 3-methyl-butanoate |
| 2-14 | 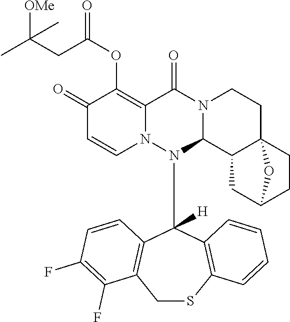 [SMILES: COC(C)(C)CC(=O)Oc1c2n(ccc1=O)N([C@@H]1c3ccccc3SCc3c1ccc(F)c3F)[C@@H]1[C@H]3C[C@@H]4CC[C@@]3(CCN1C2=O)O4] | (2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl 3-methoxy-3-methylbutanoate |
| 2-15 | 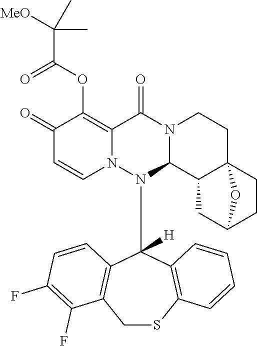 [SMILES: COC(C)(C)C(=O)Oc1c2n(ccc1=O)N([C@@H]1c3ccccc3SCc3c1ccc(F)c3F)[C@@H]1[C@H]3C[C@@H]4CC[C@@]3(CCN1C2=O)O4] | (2S,4aS,14aR,14bR)-14-((S)-7,8-difluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl 2-methoxy-2-methylpropanoate |
| 2-16 | 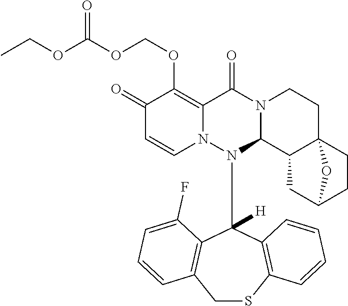 [SMILES: CCOC(=O)OCOc1c2n(ccc1=O)N([C@@H]1c3ccccc3SCc3cccc(F)c31)[C@@H]1[C@H]3C[C@@H]4CC[C@@]3(CCN1C2=O)O4] | ethyl ((((2S,4aS,14aR,14bR)-14-((S)-10- fluoro-6,11-dihydrodibenzo[b,e]thiepin-11- yl)-8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)oxy)methyl) carbonate |
| 2-17 | 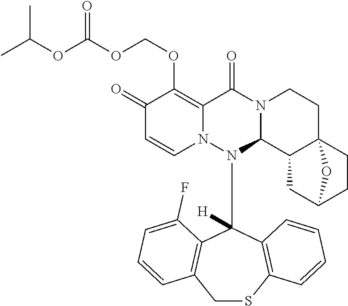 [SMILES: CC(C)OC(=O)OCOc1c2n(ccc1=O)N([C@@H]1c3ccccc3SCc3cccc(F)c31)[C@@H]1[C@H]3C[C@@H]4CC[C@@]3(CCN1C2=O)O4] | (((2S,4aS,14aR,14bR)-14-((S)-10-fluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)-oxy)methyl isopropyl carbonate |
| 2-18 | 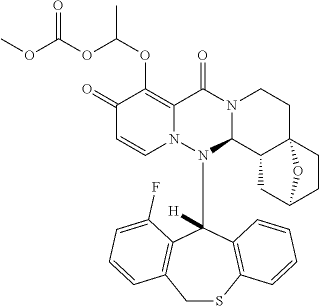 [SMILES: COC(=O)OC(C)Oc1c2n(ccc1=O)N([C@@H]1c3ccccc3SCc3cccc(F)c31)[C@@H]1[C@H]3C[C@@H]4CC[C@@]3(CCN1C2=O)O4] | 1-(((2S,4aS,14aR,14bR)-14-((S)-10-fluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)-oxy)ethyl methyl carbonate |
| 2-19 | 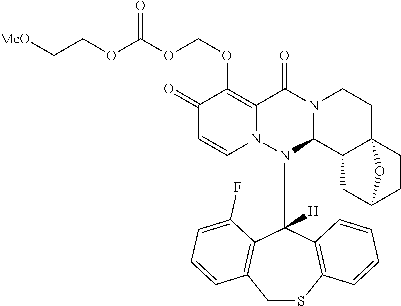 [SMILES: COCCOC(=O)OCOc1c2n(ccc1=O)N([C@@H]1c3ccccc3SCc3cccc(F)c31)[C@@H]1[C@H]3C[C@@H]4CC[C@@]3(CCN1C2=O)O4] | (((2S,4aS,14aR,14bR)-14-((S)-10-fluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)oxy)methyl (2-methoxyethyl) carbonate |
| 2-20 | 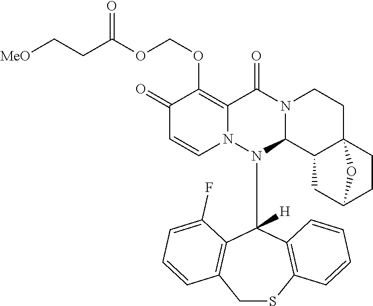 [SMILES: COCCC(=O)OCOc1c2n(ccc1=O)N([C@@H]1c3ccccc3SCc3cccc(F)c31)[C@@H]1[C@H]3C[C@@H]4CC[C@@]3(CCN1C2=O)O4] | (((2S,4aS,14aR,14bR)-14-((S)-10-fluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)-oxy)methyl 3- methoxypropanoate |
| 2-21 | 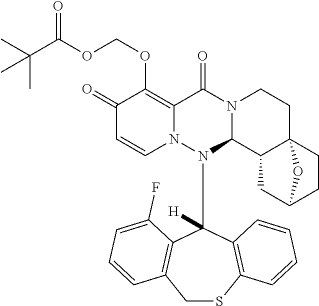 [SMILES: CC(C)(C)C(=O)OCOc1c2n(ccc1=O)N([C@@H]1c3ccccc3SCc3cccc(F)c31)[C@@H]1[C@H]3C[C@@H]4CC[C@@]3(CCN1C2=O)O4] | (((2S,4aS,14aR,14bR)-14-((S)-10-fluoro- 6,11-dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- yl)-oxy)methyl pivalate |
| 2-22 | 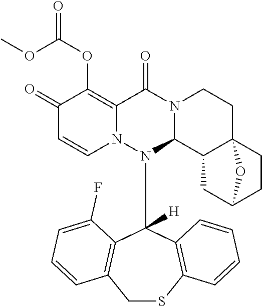 [SMILES: COC(=O)Oc1c2n(ccc1=O)N([C@@H]1c3ccccc3SCc3cccc(F)c31)[C@@H]1[C@H]3C[C@@H]4CC[C@@]3(CCN1C2=O)O4] | (2S,4aS,14aR,14bR)-14-((S)-10-fluoro-6,11- dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- methyl carbonate |
| 2-23 | 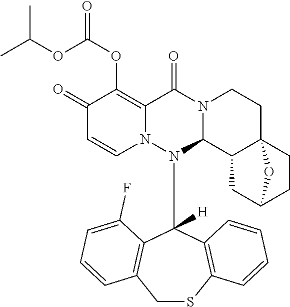 [SMILES: CC(C)OC(=O)Oc1c2n(ccc1=O)N([C@@H]1c3ccccc3SCc3cccc(F)c31)[C@@H]1[C@H]3C[C@@H]4CC[C@@]3(CCN1C2=O)O4] | (2S,4aS,14aR,14bR)-14-((S)-10-fluoro-6,11- dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- isopropyl carbonate |
| 2-24 | 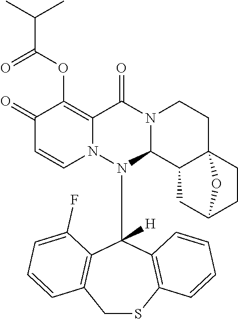 [SMILES: CC(C)C(=O)Oc1c2n(ccc1=O)N([C@@H]1c3ccccc3SCc3cccc(F)c31)[C@@H]1[C@H]3C[C@@H]4CC[C@@]3(CCN1C2=O)O4] | (2S,4aS,14aR,14bR)-14-((S)-10-fluoro-6,11- dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- isobutyrate |
| 2-25 | 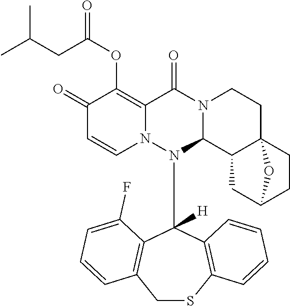 [SMILES: CC(C)CC(=O)Oc1c2n(ccc1=O)N([C@@H]1c3ccccc3SCc3cccc(F)c31)[C@@H]1[C@H]3C[C@@H]4CC[C@@]3(CCN1C2=O)O4] | (2S,4aS,14aR,14bR)-14-((S)-10-fluoro-6,11- dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- 3-methylbutanoate |
| 2-26 | 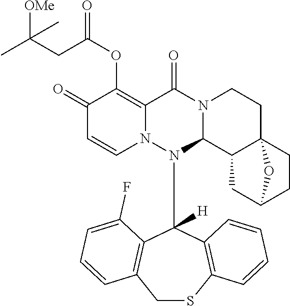 [SMILES: COC(C)(C)CC(=O)Oc1c2n(ccc1=O)N([C@@H]1c3ccccc3SCc3cccc(F)c31)[C@@H]1[C@H]3C[C@@H]4CC[C@@]3(CCN1C2=O)O4] | (2S,4aS,14aR,14bR)-14-((S)-10-fluoro-6,11- dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- 3-methoxy-3- methylbutanoate |
| 2-27 | 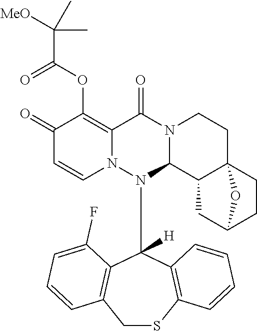 [SMILES: COC(C)(C)C(=O)Oc1c2n(ccc1=O)N([C@@H]1c3ccccc3SCc3cccc(F)c31)[C@@H]1[C@H]3C[C@@H]4CC[C@@]3(CCN1C2=O)O4] | (2S,4aS,14aR,14bR)-14-((S)-10-fluoro-6,11- dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- 2-methoxy-2-methylpropanoate |
| 2-28 | 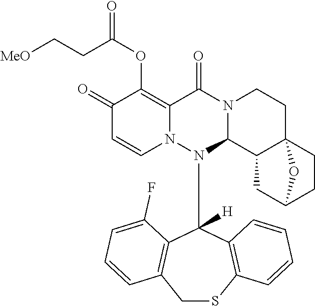 [SMILES: COCCC(=O)Oc1c2n(ccc1=O)N([C@@H]1c3ccccc3SCc3cccc(F)c31)[C@@H]1[C@H]3C[C@@H]4CC[C@@]3(CCN1C2=O)O4] | (2S,4aS,14aR,14bR)-14-((S)-10-fluoro-6,11- dihydro-dibenzo[b,e]thiepin-11-yl)- 8,10-dioxo-1,3,4,5,6,8,10,14,14a,14b- decahydro-2H-2,4a-epoxypyrido- [1′,2′:1,6][1,2,4]triazino[3,4-a]isoquinolin-9- 3-methoxypropanoate |

### EMBODIMENTS

In embodiments 1A-17 below, the present disclosure includes:

1A. In embodiment 1A, the compound of Formula (IA) or a pharmaceutically acceptable salt thereof is as described in the Summary above.

1. In embodiment 1, the compound of Formula (I), or a pharmaceutically acceptable salt thereof, is as described in the Summary above.

2. In embodiment 2, the compound of any one of embodiments 1A and 1, or a pharmaceutically acceptable salt thereof, has the structure of formula (II):

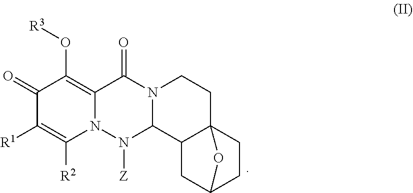

In a first sub-embodiment of embodiment 2, the compound of formula (II), or a pharmaceutically acceptable salt thereof, has the structure:

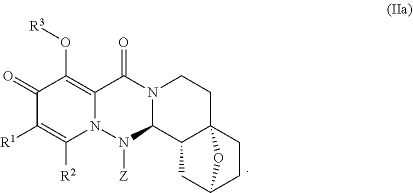

3. In embodiment 3, the compound of any one of embodiments 1A and 1, or a pharmaceutically acceptable salt thereof, has the structure of formula (III):

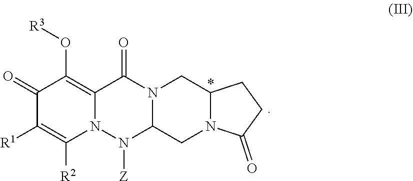

In a first sub-embodiment of embodiment 3, the compound or a pharmaceutically acceptable salt thereof is wherein the stereochemistry at *C is (S). In a second sub-embodiment of embodiment 3, the compound or a pharmaceutical salt thereof is wherein the stereochemistry at *C is (R).

4. In embodiment 4, the compound of any one of embodiments 1A and 1, or a pharmaceutically acceptable salt thereof, has the structure of formula (IV):

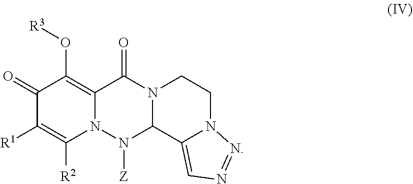

5. In embodiment 5, the compound of any one of embodiments 1A and 1 to 4 and subembodiments contained therein, or a pharmaceutically acceptable salt thereof, is where Z is a ring of formula (i). Within the groups in embodiment 5, in a first subembodiment, the compound, or a pharmaceutically acceptable salt thereof, is where X is S. Within the groups in embodiment 5, in a second subembodiment, the compound, or a pharmaceutically acceptable salt thereof, is where X is S(O). Within the groups in embodiment 5, in a third subembodiment, the compound, or a pharmaceutically acceptable salt thereof, is where X is S(O)₂. Within the groups in embodiment 5, in a first subembodiment, the compound, or a pharmaceutically acceptable salt thereof, is where X is O.

Within the groups in embodiment 5, the compound, or a pharmaceutically acceptable salt thereof, is where Z is a ring of formula (i) substituted with 1, 2, or 3 (preferably 1 or 2) halo. Within the groups in embodiment 5, the compound, or a pharmaceutically acceptable salt thereof, is where Z is a ring of formula (i) substituted with 1, 2, or 3 (preferably 1 or 2) fluoro.

Within the groups in embodiment 5, the compound, or a pharmaceutically acceptable salt thereof, is where Z is a ring of formula:

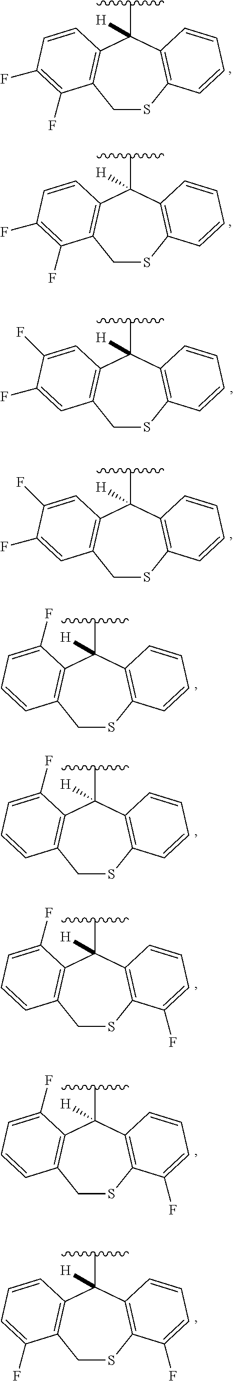

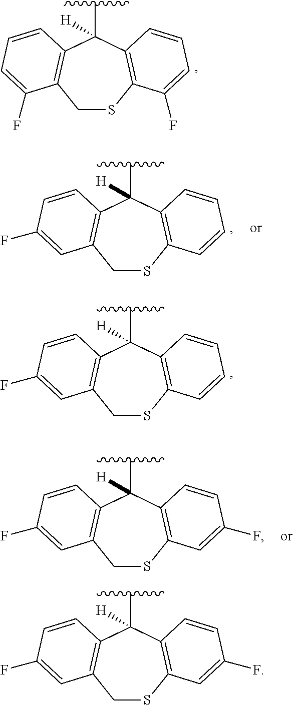

Within the groups in embodiment 5, the compound, or a pharmaceutically acceptable salt thereof, is where Z is a ring of formula:

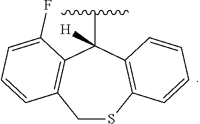 [SMILES: *[C@@H]1c2ccccc2SCc2cccc(F)c21]

Within the groups in embodiment 5, the compound, or a pharmaceutically acceptable salt thereof, is where Z is a ring of formula:

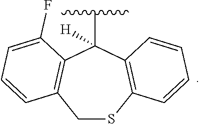 [SMILES: *[C@H]1c2ccccc2SCc2cccc(F)c21]

Within the groups in embodiment 5, the compound, or a pharmaceutically acceptable salt thereof, is where Z is a ring of formula:

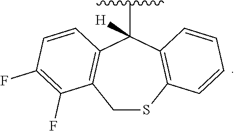 [SMILES: *[C@@H]1c2ccccc2SCc2c1ccc(F)c2F]

Within the groups in embodiment 5, the compound, or a pharmaceutically acceptable salt thereof, is where Z is a ring of formula:

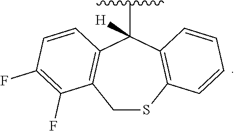 [SMILES: *[C@@H]1c2ccccc2SCc2c1ccc(F)c2F]

6. In embodiment 6, the compound of any one of embodiments 1A and 1 to 4 and subembodiments contained therein, or a pharmaceutically acceptable salt thereof, is where Z is a ring of formula (ii). Within the groups in embodiment 5, in a first subembodiment, the compound, or a pharmaceutically acceptable salt thereof, is where Ar¹ and Ar² are each phenyl, where the phenyl are independently optionally substituted with one, two, or three substituents independently selected from halo, alkyl, haloalkyl, alkoxy, haloalkoxy, alkynyl, and cyano.

Within the groups in embodiment 6, the compound, or a pharmaceutically acceptable salt thereof, is where Z is a ring of formula (ii) substituted with 1, 2, or 3 (preferably 1 or 2) halo. Within the groups in embodiment 6, the compound, or a pharmaceutically acceptable salt thereof, is where Z is a ring of formula (ii) substituted with 1, 2, or 3 (preferably 1 or 2) fluoro.

7. In embodiment 7, the compound of any one of embodiments 1A and 1 to 6 and subembodiments contained therein, or a pharmaceutically acceptable salt thereof, is where R¹ and R² are independently hydrogen, methyl, fluoro, hydroxy, trifluoromethyl, or trifluoromethoxy. In a first subembodiment of embodiment 7, the compound, or pharmaceutically acceptable salt thereof, is where R¹ and R² are each hydrogen.

8. In embodiment 8, the compound of any one of embodiments 1A and 1 to 7 and sub-embodiments contained therein, or a pharmaceutically acceptable salt thereof, is where R³ is hydrogen.

9. In embodiment 9, the compound of any one of embodiments 1A and 1 to 7 and sub-embodiments contained therein, or a pharmaceutically acceptable salt thereof, is where R³ is —C(O)R⁶ where R⁶ is alkyl optionally substituted with alkoxy, preferably R⁶ is methyl, ethyl, isopropyl, 2-methylpropyl, 2-methoxyethyl, 2-methoxy-2-methylethyl, or 2-methoxy-2-methylpropyl.

10. In embodiment 10, the compound of any one of embodiments 1A and 1 to 7 and sub-embodiments contained therein, or a pharmaceutically acceptable salt thereof, is where R³ is —C(O)—O—R⁷ where R⁷ is alkyl, preferably R⁷ is methyl, ethyl, isopropyl, 2-methylpropyl, 2-methoxyethyl, 2-methoxy-2-methylethyl, or 2-methoxy-2-methylpropyl.

11. In embodiment 11, the compound of any one of embodiments 1A and 1 to 7 and sub-embodiments contained therein, or a pharmaceutically acceptable salt thereof, is where R³ is —CH(R⁹)—O—C(O)R¹⁰ where R⁹ is hydrogen or alkyl and R¹⁰ is alkyl optionally substituted with alkoxy, preferably R³ is —CH₂—OC(O)methyl, —CH₂—OC(O)-(2-methoxyethyl), or —CH₂—OC(O)-tert-butyl. In a subembodiment of embodiment 11, R³ is —CH(R⁹)—O—C(O)R¹⁰ where R⁹ is hydrogen or alkyl and R¹⁰ is alkyl, preferably R³ is —CH₂—OC(O)-methyl or —CH₂—OC(O)-tert-butyl.

12. In embodiment 12, the compound of any one of embodiments 1A and 1 to 7 and sub-embodiments contained therein, or a pharmaceutically acceptable salt thereof, is where R³ is

—CH(R¹²)—O—C(O)—OR¹³ where R¹² is hydrogen or alkyl and R¹³ is alkyl optionally substituted with alkoxy, preferably R³ is —CH₂—OC(O)O-methyl, —CH(CH₃)—OC(O)O-methyl, —CH₂—OC(O)O-ethyl, —CH₂—OC(O)O-isopropyl, or —CH₂—OC(O)O-(2-methoxyethyl).

13. In embodiment 13, the compound of any one of embodiments 1A, 1, 5 to 12 and sub-embodiments contained therein or a pharmaceutically acceptable salt thereof is where R⁴ and R⁵ together with the atoms to which they are attached form a ring of formula (a) wherein the ring of formula (a) can optionally be substituted with one or two substituents independently selected from alkyl, alkoxy, hydroxy, halo, haloalkyl, haloalkoxy, and cyano.

14. In embodiment 16, the compound of any one of embodiments 1A, 1, 5 to 12 and sub-embodiments contained therein or a pharmaceutically acceptable salt thereof is where R⁴ and R⁵ together with the atoms to which they are attached form a ring of formula (a) wherein the ring of formula (a) can optionally be substituted with one or two substituents independently selected from alkyl, alkoxy, hydroxy, halo, haloalkyl, haloalkoxy, and cyano, preferably one or two substituents independently selected from methyl, hydroxy, methoxy, or fluoro.

15. In embodiment 14, the compound of any one of embodiments 1A, 1, 5 to 12 and sub-embodiments contained therein or a pharmaceutically acceptable salt thereof is where R⁴ and R⁵ together with the atoms to which they are attached form a ring of formula (b) wherein the ring of formula (b) can optionally be substituted with one or two substituents independently selected from alkyl, alkoxy, hydroxy, halo, haloalkyl, haloalkoxy, and cyano, preferably one or two substituents independently selected from methyl, hydroxy, methoxy, or fluoro.

16. In embodiment 16, the compound of any one of embodiments 1A, 1, 5 to 12 and sub-embodiments contained therein or a pharmaceutically acceptable salt thereof is where R⁴ and R⁵ together with the atoms to which they are attached form a ring of formula (c) wherein the ring of formula (c) can optionally be substituted with one or two substituents independently selected from alkyl, alkoxy, hydroxy, halo, haloalkyl, haloalkoxy, and cyano, preferably one or two substituents independently selected from methyl, hydroxy, methoxy, or fluoro.

17. In embodiment 16, the compound of any one of embodiments 1A, 1, 5 to 12 and sub-embodiments contained therein or a pharmaceutically acceptable salt thereof is where R⁴ and R⁵ together with the atoms to which they are attached form a ring of formula (d) wherein the ring of formula (d) can optionally be substituted with one or two substituents independently selected from alkyl, alkoxy, hydroxy, halo, haloalkyl, haloalkoxy, and cyano, preferably one or two substituents independently selected from methyl, hydroxy, methoxy, or fluoro.

It is understood that the embodiments set forth above include all combination of embodiments and sub-embodiments listed therein. For example, the R³ group listed in embodiment 12 and one or more preferred groups therein, can independently combine with one or more of the embodiments IA and 1-7 and/or sub-embodiments contained therein.

### General Synthetic Scheme

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formulae (IA) and (I) where R¹, R², R³, R⁴ and R⁵ are as defined in the summary can be prepared as illustrated and described in Scheme 1 below.

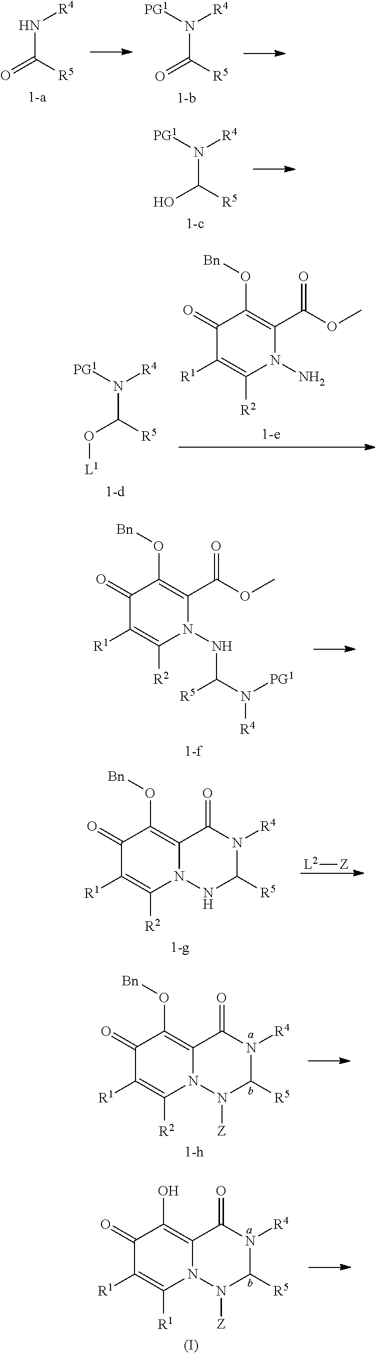

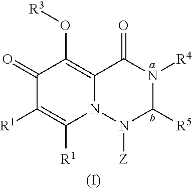

Protection of the nitrogen atom in a compound of formula 1-a where R⁴ and R⁵ together with the atoms to which they are attached form a ring of formula (a) can be carried out by first deprotonation of NH group with a base such as n-butyl lithium, followed by treatment with a suitable protecting group such as a haloformic acid alkyl e.g., allylchloroformate to provide compound of formula 1-b where PG¹ is a protecting group. Treatment of compound 1-b with a reducing reagent such as lithium diisobutylaluminum hydride provides a compound of formula 1-c. Compound 1-c can be converted into a compound of formula 1-d where L¹ is an alkyl group by treating it with an acid such as p-toluene sulfonic acid or methane sulfonic acid in alcohol solvent.

Reaction of a compound of formula 1-d with a compound of formula 1-e where Bn is benzyl or other suitable protecting group and R¹ and R² are as defined in the Summary, in the presence of Lewis acid such as SnCl₄ to provide compound of formula 1-f. Removal of the amino protecting group in 1-f, followed by cyclization of the resulting amine provides a compound of formula 1-g.

Coupling of compound 1-g with a compound of formula L²-Z where L² is a leaving group such as halo, hydroxyl, or acetate and Z is as defined in the Summary, in the presence of T3P, methanesulfonic acid or para-toluenesulfonic acid provides a compound of formula 1-h. Removal of the benzyl group in 1-h by methods well known in the art provides a compound of Formula (I) where R³ is hydrogen. Compounds of Formula (I) where R³ is hydrogen can be converted to corresponding compounds of Formula (I) where R³ is other than hydrogen by methods well known in the art. Detailed synthesis of a compound of formula 1-g is provided in Synthetic Example 1 below.

Alternatively, compounds of Formula (I) where R¹, R², R³, R⁴ and R⁵ are as defined in the summary can be prepared as illustrated and described in Scheme 2 below.

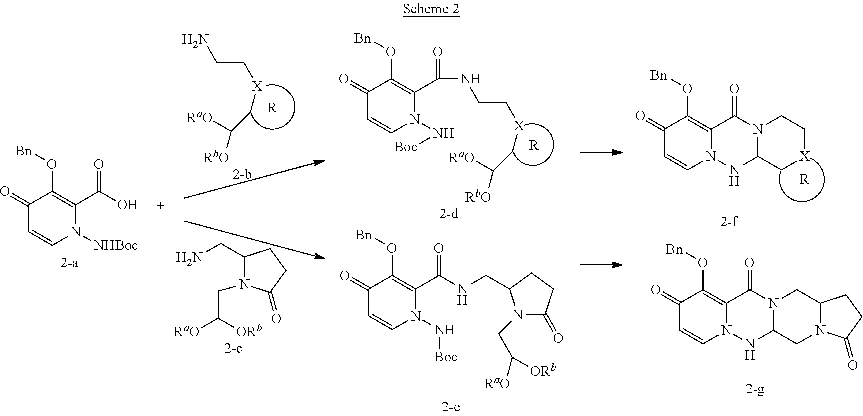

Treatment of compound of formula 2a with a compound of formula 2-b where

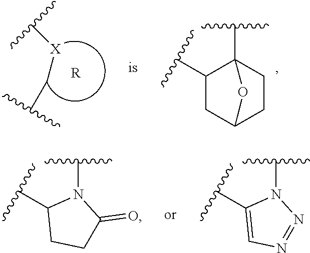

or with a compound of formula 2-c where each R^{a} and R^{b} are alkyl, in the presence of an amide coupling reagent such HATU provides a compound of formula 2-d or 2-e, respectively. Compound of formula 2-d and 2-e can be converted to compounds 2-f and 2-g respectively, by treatment with an acid such as para-toluenesulfonic acid, methanesulfonic acid in a mixture of water and organic solvent such as CH₃CN, followed by cyclization of the resulting aldehydes by methods well known in the art.
Removal of the benzyl group in compounds 2f and 2g, then provides compounds of Formula (I) where R³ is hydrogen which can then be converted to compounds of Formula (I) where R³ is other than hydrogen.

### Utility

The compounds of Formula (I), in free form or in salt form inhibit replication of orthomyxovirus and are therefore indicated for therapy or for use as research chemicals, e.g. as tool compounds such as for the study of replication of an orthomyxovirus, preferably Influenza A, Influenza B or Influenza C, more preferably Influenza A and B. Accordingly, compounds of Formula (I) or a salt thereof are useful in the treatment of an infection caused by an orthymyxovirus, preferably Influenza A, Influenza B or Influenza C, more preferably Influenza A and B, especially in human subjects. In some embodiments, the subject to be treated is a human having or at risk of contracting an influenza viral infection. For example, subjects having pre-existing conditions such as asthma or COPD that can be exacerbated by an influenza infection may be treated with the methods or compounds of Formula (I) or a salt thereof before exhibiting symptoms of an influenza infection. In other embodiments, the subject for treatment by the methods and compounds of Formula (I) or a salt thereof is one diagnosed as having symptoms consistent with an influenza infection. In other embodiments, the subject may be a human who has been tested with known diagnostic methods such as a Rapid Influenza Diagnostic Test (RIDT) or Reverse Transcriptase PCT (RT-PCR) methods to detect the presence of influenza virus, and found to be infected with influenza, regardless of the presence of typical influenza symptoms.

In another embodiment, provided is a method of treating a disease which is caused by an orthomyxovirus, comprising administration of a therapeutically effective amount of a compound of Formula (I) or any of the embodiments thereof described herein to a subject in need of such treatment. In some embodiments, the disease caused by orthomyxovirus selected from Influenza A, Influenza B, and Influenza C, preferably Influenza A and B.

### Pharmaceutical Compositions

In general, the compounds of Formula (I) or a pharmaceutically acceptable salt thereof will be administered in a therapeutically effective amount by any of the accepted modes of administration for agents that serve similar utilities. Therapeutically effective amounts of compounds this disclosure may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the patient, the potency of the compound being utilized, the route and form of administration, and other factors.

The pharmaceutical composition can be formulated for particular routes of administration such as oral administration, parenteral administration, and rectal administration, and the like. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Suitable compositions for oral administration include a compound of Formula (I) or a pharmaceutically acceptable salt thereof in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may also contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate: granulating and disintegrating agents, for example, corn starch, or alginic acid; binding agents, for example, starch, gelatin or acacia; and lubricating agents, for example magnesium stearate, stearic acid or talc. The tablets are uncoated or coated (e.g., enteric coated) by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Certain injectable compositions are aqueous isotonic solutions or suspensions, and suppositories are advantageously prepared from fatty emulsions or suspensions. These compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. These compositions are prepared according to conventional mixing, granulating or coating methods, respectively, and contain about 0.1-75%, or contain about 1-50%, of the active ingredient.

Compositions for transdermal application include a compound of Formula (I) or a pharmaceutically acceptable salt thereof with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Compositions for topical application, e.g., to the skin and eyes, include aqueous solutions, suspensions, ointments, creams, gels or sprayable formulations, e.g., for delivery by aerosol or the like. Such topical delivery systems may pertain to an inhalation or to an intranasal application that may be suitable for use to treat influenza, for example, and may contain solubilizers, stabilizers, tonicity enhancing agents, buffers and preservatives. They may be conveniently delivered in the form of a dry powder (either alone, as a mixture, for example a dry blend with lactose, or a mixed component particle, for example with phospholipids) from a dry powder inhaler or an aerosol spray presentation from a pressurized container, pump, spray, atomizer or nebulizer, with or without the use of a suitable propellant.

The present disclosure further provides anhydrous pharmaceutical compositions and dosage forms comprising the compounds of Formula (I) or a pharmaceutically acceptable salt thereof as active ingredients, since water may facilitate the degradation of certain compounds. Anhydrous pharmaceutical compositions and dosage forms can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary, kits. Examples of suitable packaging include, but are not limited to, hermetically sealed foils, plastics, unit dose containers (e. g., vials), blister packs, and strip packs.

Also provided are pharmaceutical compositions and dosage forms that comprise one or more agents that reduce the rate by which the compound of Formula (I) as an active ingredient will decompose. Such agents, which are referred to herein as “stabilizers,” include, but are not limited to, antioxidants such as ascorbic acid, pH buffers, or salt buffers, etc.

The compound of Formula (I) or a salt thereof may be administered either simultaneously or sequentially with one or more therapeutic co-agent(s). The compound of Formula (I) or a salt thereof may be administered separately, by the same or different route of administration, or together in the same pharmaceutical composition as the co-agent(s). Suitable co-agents for use with the compounds of the invention include antivirals active on influenza viruses, such as neuraminidase inhibitors including oseltamivir, peramivir, zanamivir and laninamivir, laninamivir octanoate, and adamantanes such as amantadine and rimantadine. Additional co-agents for use in these methods include an M2 protein inhibitor, a polymerase inhibitor, a PB2 inhibitor, favipiravir, fludase, ADS-8902, beraprost, Neugene®, ribavirin, compound with CAS Reg. No. 1422050-75-6, VX-787, Flu Mist Quadrivalent®, Fluarix® Quadrivalent, Fluzone® Quadrivalent, Flucelvax® and FluBlok®.

In one embodiment, provided is a product comprising a compound of Formula (I) or a salt thereof and at least one other therapeutic co-agent for simultaneous, separate or sequential use in therapy. In one embodiment, the therapy is the treatment of a viral infection caused by an orthomyxovirus, preferably Influenza A, Influenza B or Influenza C, more preferably Influenza A and B. Products provided as a combined preparation include a composition comprising a compound of formula (I) and at least one of the other therapeutic co-agent(s) together in the same pharmaceutical composition, or the compound of formula (I) and at least one other therapeutic co-agent(s) in separate form, e.g. in the form of a kit for use to treat a subject by the methods described herein. Suitable co-agents include antivirals active on influenza viruses, such as neuraminidase inhibitors including oseltamivir, peramivir, zanamivir and laninamivir, and adamantanes such as amantadine and rimantadine. Optionally, the pharmaceutical composition may comprise a pharmaceutically acceptable carrier, as described above.

In another embodiment, provided is a kit comprising two or more separate pharmaceutical compositions, at least one of which contains a compound of formula (I) or a salt thereof. The other pharmaceutical composition may contain one of the suitable co-agents. In one embodiment, the kit comprises means for separately retaining said compositions, such as a container, divided bottle, or divided foil packet. An example of such a kit is a blister pack, as typically used for the packaging of tablets, capsules and the like. The kit may be used for administering different dosage forms, for example, oral and parenteral, for administering the separate compositions at different dosage intervals, or for titrating the separate compositions against one another. To assist compliance, the kit typically comprises directions for administration.

Accordingly, also provided is the use of a compound of Formula (I) or a salt thereof for treating a viral infection caused by an orthomyxovirus, particularly influenza, which may be Influenza A, Influenza B or Influenza C, wherein the medicament is prepared for administration with a therapeutic co-agent.

### EXAMPLES

The following preparations of intermediates (References) compounds of Formula (I) are given to enable those skilled in the art to more clearly understand and to practice the present disclosure. They should not be considered as limiting the scope of the disclosure, but merely as being illustrative and representative thereof.

### Example 1

### Synthesis of (2R,4aR,14aS,14bS)-14-((R)-7,8-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione[1] and (2S,4aS,14aR,14bR)-14-((R)-7,8-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[2]

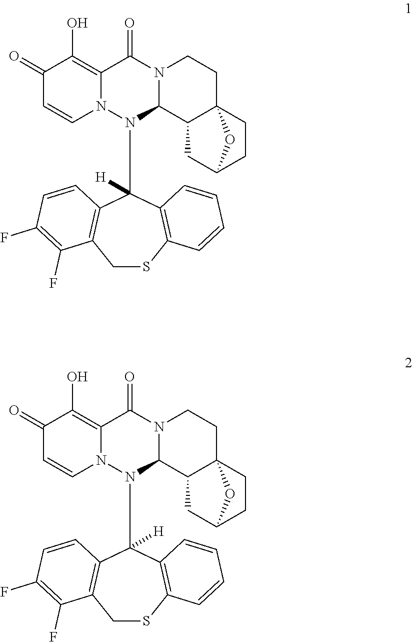 [SMILES: O=C1c2c(O)c(=O)ccn2N([C@@H]2c3ccccc3SCc3c2ccc(F)c3F)[C@@H]2[C@H]3C[C@@H]4CC[C@@]3(CCN12)O4 ; O=C1c2c(O)c(=O)ccn2N([C@H]2c3ccccc3SCc3c2ccc(F)c3F)[C@@H]2[C@H]3C[C@@H]4CC[C@@]3(CCN12)O4]

### Step 1: N-(2,4-dimethoxybenzyl)-2-(furan-2-yl)ethan-1-amine

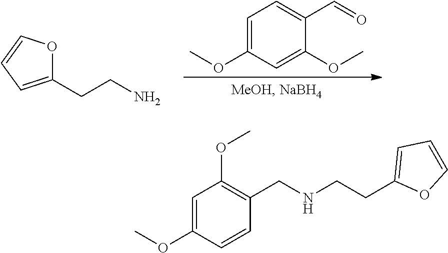

Into a 2.0 L 4-necked round-bottom flask purged and maintained under an inert atmosphere of nitrogen, was placed MeOH (730 mL), 2-(furan-2-yl)ethan-1-amine (73.0 g, 656.8 mmol, 1.0 equiv.), and 2,4-dimethoxybenzaldehyde (109 g, 655.9 mmol, 1.0 equiv.). The resulting solution was stirred for 30 min at room temperature. NaBH₄ (25 g, 660.9 mmol, 1.0 equiv.) was added in portions with stirring at 0° C. over 30 mins. The resulting mixture was stirred for additional 2 hr at room temperature. The reaction was quenched with water and then concentrated under vacuum. The residue was diluted with water and extracted with ethyl acetate. The combined organic layers were dried over anhydrous sodium sulfate and concentrated under vacuum to afford 70 g (crude) of the title compound as dark yellow oil. MS (ES, m/z): [M+1]⁺=262.1.

### Step 2: N-(2,4-dimethoxybenzyl)-N-(2-(furan-2-yl)ethyl)acrylamide

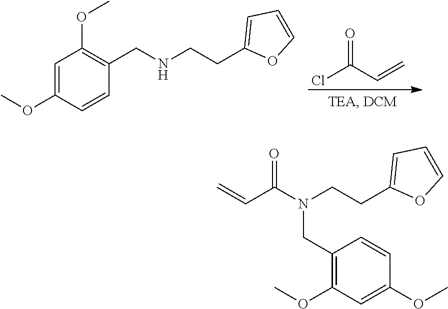

Into a 1 L 4-necked round-bottom flask purged and maintained with an inert atmosphere of nitrogen, was placed [(2,4-dimethoxyphenyl)methyl][2-(furan-2-yl)ethyl]amine (65.0 g, 248.7 mmol, 1.0 equiv.), DCM (650 mL) and TEA (52.0 g, 513.9 mmol, 2.10 equiv.). Prop-2-enoyl chloride (35.0 g, 386.7 mmol, 1.55 equiv.) was added dropwise with stirring at 0° C. After stirring at 0° C. for 30 mins the reaction was quenched with water. The resulting mixture was extracted with DCM and the combined organic layers were washed with water and brine, dried over anhydrous sodium sulfate and concentrated to afford 40 g (50% yield) of the title compound as yellow oil. MS (ES, m/z): [M+1]⁺=316.2.

### Step 3: rac-(4aR,7R,8aS)-2-(2,4-dimethoxybenzyl)-3,4,8,8a-tetrahydro-2H-4a,7-epoxyisoquinolin-1(7H)-one

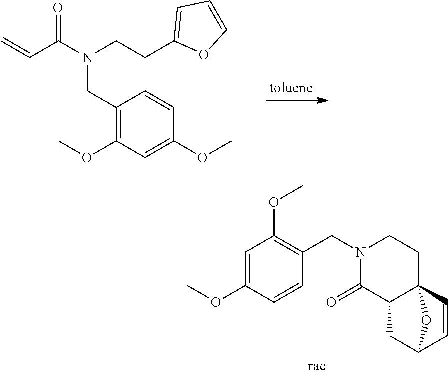 [SMILES: C=CC(=O)N(CCc1ccco1)Cc1ccc(OC)cc1OC ; COc1ccc(CN2CC[C@@]34C=C[C@@H](C[C@@H]3C2=O)O4)c(OC)c1]

Into a 2.0 L round-bottom flask purged and maintained with an inert atmosphere of nitrogen, were placed N-[(2,4-dimethoxyphenyl)methyl]-N-[2-(furan-2-yl)ethyl]prop-2-enamide (40.0 g, 126.8 mmol) and toluene (1200 mL). The resulting solution was stirred for 10 hrs at 110° C. and then concentrated. The residue was applied onto a silica gel column and eluted with ethyl acetate/petroleum ether to afford 32 g (80% yield) of the title compound as light yellow oil. MS (ES, m/z): [M+1]⁺=316.2.

### Step 4: rac-(4aS,7S,8aS)-2-(2,4-dimethoxybenzyl)hexahydro-2H-4a,7-epoxyisoquinolin-1(5H)-one

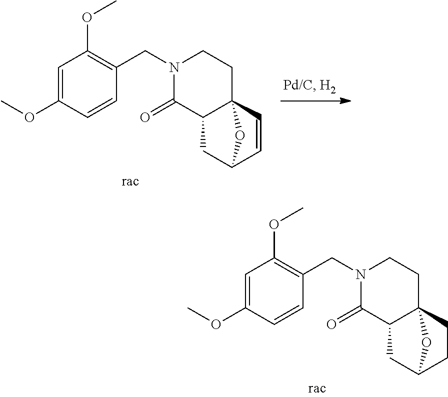 [SMILES: COc1ccc(CN2CC[C@@]34C=C[C@@H](C[C@@H]3C2=O)O4)c(OC)c1 ; COc1ccc(CN2CC[C@@]34CC[C@@H](C[C@@H]3C2=O)O4)c(OC)c1]

Into a 1 L round-bottom flask purged and maintained with an inert atmosphere of nitrogen, was placed rac-(4aR,7R,8aS)-2-(2,4-dimethoxybenzyl)-3,4,8,8a-tetrahydro-2H-4a,7-epoxyisoquinolin-1(7H)-one (32.0 g, 101.5 mmol, 1.0 equiv.), MeOH (600 mL), and Pd/C (3 g, 10% on activated carbon). The resulting mixture was stirred for 1 h under H₂ atmosphere (2-3 atm) at room temperature. The mixture was filtered and the filtrate was concentrated under vacuum to afford 30 g (93%) of the title compound as colorless oil. (ES, m/z): [M+1]⁺=318.2.

### Step 5: rac-(4aS,7S,8aS)-hexahydro-2H-4a,7-epoxyisoquinolin-1(5H)-one

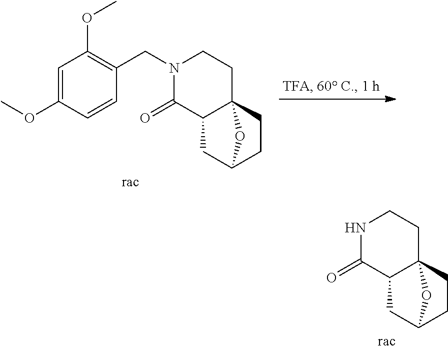 [SMILES: COc1ccc(CN2CC[C@@]34CC[C@@H](C[C@@H]3C2=O)O4)c(OC)c1 ; O=C1NCC[C@@]23CC[C@@H](C[C@H]12)O3]

Into a 50 mL 3-necked round-bottom flask purged and maintained with an inert atmosphere of nitrogen, were placed rac-(4aS,7S,8aS)-2-(2,4-dimethoxybenzyl)hexahydro-2H-4a,7-epoxyisoquinolin-1(5H)-one (1.5 g, 4.75 mmol, 1.0 equiv.) and TFA (20 mL). The reaction mixture was stirred for 1 h at 60° C. and then concentrated. The residue was diluted with water. The pH of the aqueous solution was adjusted to 7 with saturated sodium bicarbonate solution. The resulting solution was extracted with DCM. The combined organic layers were concentrated under vacuum to afford 1.3 g (crude) of the title compound as a white solid. (ES, m/z): [M+1]⁺+=168.2.

### Step 6: rac-allyl (4aS,7S,8aS)-1-oxooctahydro-2H-4a,7-epoxyisoquinoline-2-carboxylate

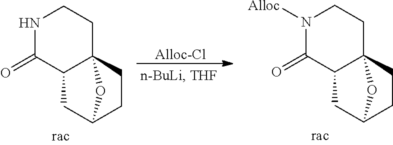

To a 50 mL 3-necked round-bottom flask purged and maintained with an inert atmosphere of nitrogen, were placed rac-(4aS,7S,8aS)-hexahydro-2H-4a,7-epoxyisoquinolin-1(5H)-one (1.30 g, 7.78 mmol, 1.0 equiv.), and THF (15 mL). n-BuLi (3.11 mL, 2.5 M in hexanes, 7.78 mmol, 1.0 equiv.) was added dropwise with stirring at −78° C. and the resulting solution was stirred for 1 h at −78° C. Prop-2-en-1-yl carbonochloridate (0.93 g, 7.72 mmol, 1.0 equiv.) was added dropwise with stirring at −78° C. and the resulting reaction mixture was stirred for 1 h at −78° C. The reaction was quenched with water and the mixture was extracted with ethyl acetate. The combined organic layers were washed with water and brine, dried over anhydrous sodium sulfate and concentrated. The residue was purified by silica gel column chromatography, eluted with ethyl acetate/petroleum ether, to give 0.75 g (63% for two steps) of the title compound. (ES, m/z): [M+1]⁺=252.2.

### Step 7: rac-allyl (4aS,7S,8aS)-1-hydroxyoctahydro-2H-4a,7-epoxyisoquinoline-2-carboxylate

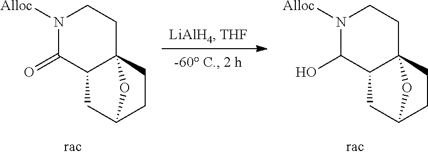

To a stirred solution of rac-allyl (4aS,7S,8aS)-1-oxooctahydro-2H-4a,7-epoxyisoquinoline-2-carboxylate (1000 mg, 3.98 mmol, 1.0 equiv.) in THF (10 mL) was added LiAlH₄ (226.56 mg, 5.9 mmol, 1.5 equiv.) in portions at −78° C. under N₂ atmosphere. The resulting mixture was stirred for 1 h at −78° C. under N₂ atmosphere and then quenched with aq. NaHCO₃ (5.0 mL) at 0° C. The resulting mixture was extracted with EtOAc and the combined organic layers were dried over anhydrous Na₂SO₄. After filtration, the filtrate was concentrated under reduced pressure to afford the title compound (800 mg). The crude product was used in the next step directly without further purification. MS (ES, m/z): [M+41+23]⁺=317.1.

### Step 8: rac-allyl (4aS,7S,8aS)-1-methoxyoctahydro-2H-4a,7-epoxyisoquinoline-2-carboxylate

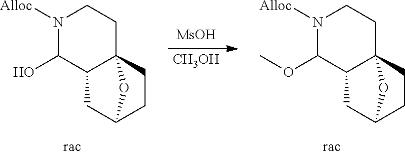

To a stirred solution of rac-allyl (4aS,7S,8aS)-1-hydroxyoctahydro-2H-4a,7-epoxy-isoquinoline-2-carboxylate (850 mg, 3.36 mmol, 1.0 equiv.) in MeOH (10 mL) was added methanesulfonic acid (32.2 mg, 0.34 mmol, 0.1 equiv.) at room temperature under N₂ atmosphere. The resulting mixture was stirred for 16 h at room temperature and then quenched with aq. NaHCO₃ solution (510 mL). The aqueous layer was extracted with EtOAc and the combined organic layers were dried over anhydrous Na₂SO₄. After filtration, the filtrate was concentrated under reduced pressure. The residue was purified by silica gel column chromatography, eluted with PE/EA (1/1), to afford the title compound (210 mg, 23.4%) as light yellow oil.

### Step 9: rac-allyl (4aS,7S,8aR)-1-((3-(benzyloxy)-2-(ethoxycarbonyl)-4-oxopyridin-1(4H)-yl)amino)octahydro-2H-4a,7-epoxyisoquinoline-2-carboxylate

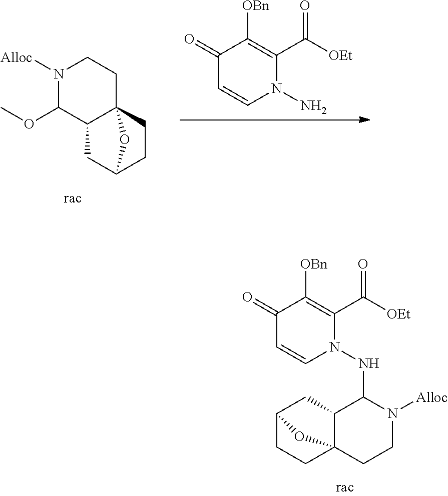

To a stirred solution of rac-allyl (4aS,7S,8aS)-1-methoxyoctahydro-2H-4a,7-epoxyisoquinoline-2-carboxylate (40 mg, 0.150 mmol, 1 equiv.) and ethyl 1-amino-3-(benzyloxy)-4-oxo-1,4-dihydropyridine-2-carboxylate (43.14 mg, 0.150 mmol, 1 equiv.) in MeCN (1 mL) was added SnCl₄ (77.96 mg, 0.299 mmol, 2 equiv.) dropwise at −20° C. under N₂ atmosphere. The reaction mixture was stirred at rt overnight and then quenched with aq. NaHCO₃ (5 mL) at −0° C. and the resulting mixture was extracted with EtOAc. The combined organic layers were dried over anhydrous Na₂SO₄. After filtration, the filtrate was concentrated under reduced pressure to give the title compound (40 mg, 51%) which was used in the next step directly without further purification. MS (ES, m/z): [M+1]⁺=524.3.

### Step 10: (2R,4aR,14aS,14bS)-9-(benzyloxy)-1,3,4,5,6,14,14a, 14b-octahydro-2H-2,4a-epoxypyrido[1′,2′:1,6][1,2,4]triazino[3,4-a]isoquinoline-8,10-dione and (2S,4aS,14aR,14bR)-9-(benzyloxy)-1,3,4,5,6,14,14a, 14b-octahydro-2H-2,4a-epoxypyrido[1′,2′:1,6][1,2,4]triazino[3,4-a]isoquinoline-8,10-dione

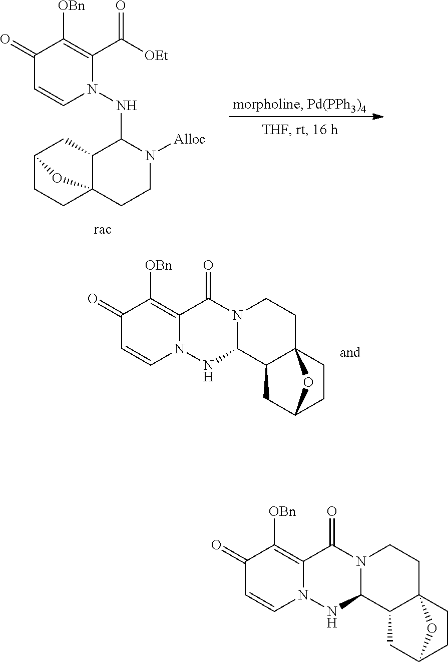

To a stirred solution of rac-allyl (4aS,7S,8aR)-1-((3-(benzyloxy)-2-(ethoxycarbonyl)-4-oxopyridin-1(4H)-yl)amino)octahydro-2H-4a,7-epoxyisoquinoline-2-carboxylate and allyl (4aR,7R,8aS)-1-((3-(benzyloxy)-2-(ethoxycarbonyl)-4-oxopyridin-1(4H)-yl)amino)octahydro-2H-4a,7-epoxyisoquinoline-2-carboxylate (800 mg, 1.5 mmol, 1.0 equiv.) and morpholine (266.2 mg, 3.1 mmol, 2.0 equiv.) in THE (20 mL) was added Pd(PPh₃)₄ (176.6 mg, 0.160 mmol, 0.1 equiv.) in portions at room temperature under N₂ atmosphere. The resulting mixture was stirred at rt for 16 hours, diluted with Et₂O (20 mL). The precipitated solids were collected by filtration and washed with Et₂O to give 500 mg of product as a yellow solid. MS (ES, m/z): [M+1]⁺=394.2. The solid was purified by chiral prep-HPLC (CHIRALPAK IA-3) to afford two enantiomers. (2R,4aR,14aS,14bS)-9-(benzyloxy)-1,3,4,5,6,14,14a,14b-octahydro-2H-2,4a-epoxypyrido[1′,2′:1,6][1,2,4]triazino[3,4-a]isoquinoline-8,10-dione trifluoroacetic acid salt salt: tR 1.47 min, (240 mg, 32.0%) and (2S,4aS,14aR,14bR)-9-(benzyloxy)-1,3,4,5,6,14,14a,14b-octahydro-2H-2,4a-epoxypyrido[1′,2′:1,6][1,2,4]triazino[3,4-a]isoquinoline-8,10-dione trifluoroacetic acid salt: tR 3.3 min, (220 mg, 29.3%).

### Step 11: (2S,4aS,14aR,14bR)-9-(benzyloxy)-14-(7,8-difluoro-6,11-dihydrodibenzo[b,e]thiepin-11-yl)-1,3,4,5,6,14,14a,14b-octahydro-2H-2,4a-epoxypyrido[1′,2′:1,6][1,2,4]triazino[3,4-a]-isoquinoline-8,10-dione

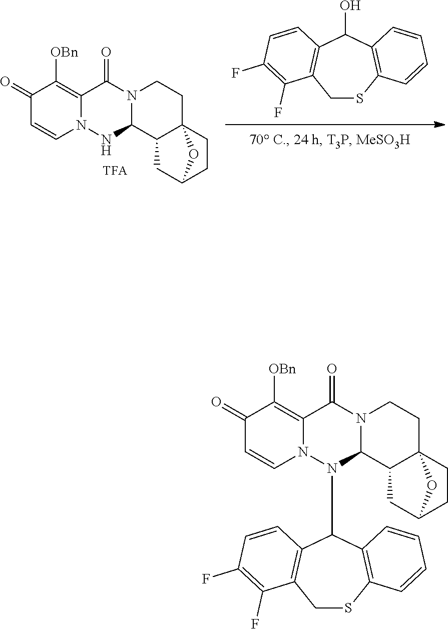

To a stirred solution of (2S,4aS,14aR,14bR)-9-(benzyloxy)-1,3,4,5,6,14,14a,14b-octahydro-2H-2,4a-epoxypyrido[1′,2′: 1,6][1,2,4]triazino[3,4-a]isoquinoline-8,10-dione trifluoroacetic acid salt (220 mg, 0.45 mmol, 1.0 equiv.) and 7,8-difluoro-6,11-dihydrodibenzo[b,e]thiepin-11-ol (119 mg, 0.45 mmol, 1.0 equiv.) in T3P (2 mL) and EtOAc (2 mL) was added methanesulfonic acid (43 mg, 0.45 mmol, 1.0 equiv.) at rt under N₂ atmosphere. The resulting mixture was stirred for 24 h at 70° C. under N₂ atmosphere. The reaction was quenched with H₂O. The resulting mixture was extracted with EtOAc and the combined organic layers were dried over anhydrous Na₂SO₄. After filtration, the filtrate was concentrated under reduced pressure to give the title product (390 mg, crude). The crude product was used in the next step directly without further purification. MS (ES, m/z): [M+1]⁺=640.3.

### Step 12: (2S,4aS,14aR,14bR)-14-((S)-7,8-difluoro-6,11-dihydrodibenzo[b,e]thiepin-11-yl)-9-hydroxy-1,3,4,5,6,14,14a,14b-octahydro-2H-2,4a-epoxypyrido[1′,2′: 1,6][1,2,4]triazino[3,4-a]isoquinoline-8,10-dione[1] and (2S,4aS,14aR,14bR)-14-((R)-7,8-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[2]

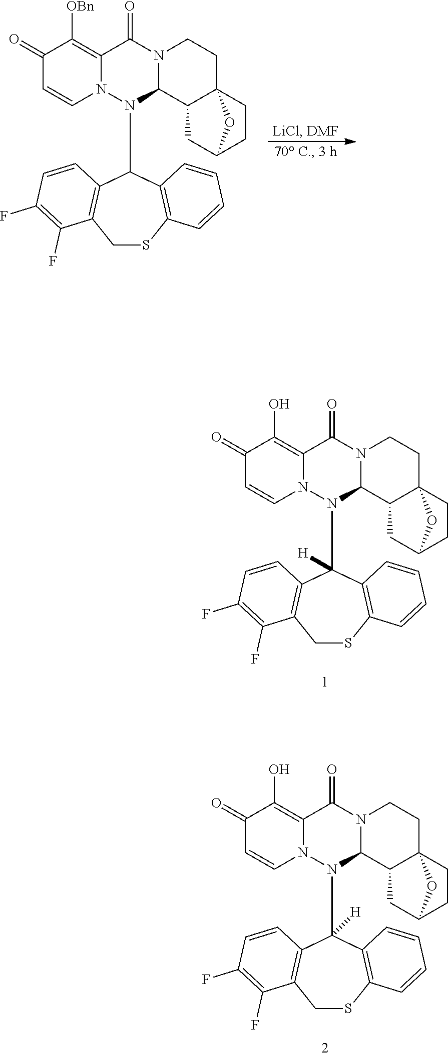

A stirred mixture of (2S,4aS,14aR,14bR)-9-(benzyloxy)-14-(7,8-difluoro-6,11-dihydrodibenzo[b,e]thiepin-11-yl)-1,3,4,5,6,14,14a,14b-octahydro-2H-2,4a-epoxypyrido[1′,2′: 1,6][1,2,4]triazino[3,4-a]isoquinoline-8,10-dione (390 mg, 0.61 mmol, 110 equiv.) and LiCl (258 mg, 6.1 mmol, 10.0 equiv.) in DMF (4.0 mL) was stirred at 70° C. for 3 h. The reaction solution was purified by Prep-HPLC to afford (2S,4aS,14aR,14bR)-14-((S)-7,8-difluoro-6,11-dihydrodibenzo[b,e]thiepin-11-yl)-9-hydroxy-1,3,4,5,6,14,14a,14b-octahydro-2H-2,4a-epoxypyrido[1′,2′: 1,6][1,2,4]triazino[3,4-a]isoquinoline-8,10-dione 111 and (2S,4aS,14aR,14bR)-14-((R)-7,8-difluoro-6,11-dihydrodibenzo[b,e]thiepin-1 l-yl)-9-hydroxy-1,3,4,5,6,14,14a,14b-octahydro-2H-2,4a-epoxypyrido[1′,2′: 1,6][1,2,4]triazino-[3,4-a]isoquinoline-8,10-dione[2] as a white solid. Compound [1]: MS (ES, m/z): [M+1]⁺=550.2. Compound [2]: MS (ES, m/z): [M+1]⁺=550.2.

### Example 3 and 4

### Synthesis of (2S,4aS,14aR,14bR)-14-((S)-10-fluoro-6,11-dihydrodibenzo[b,e]thiepin-1 l-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[3] and (2S,4aS,14aR,14bR)-14-((R)-10-fluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[4]

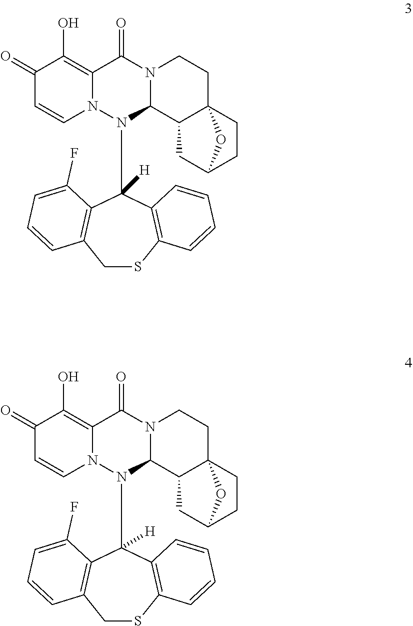 [SMILES: O=C1c2c(O)c(=O)ccn2N([C@@H]2c3ccccc3SCc3cccc(F)c32)[C@@H]2[C@H]3C[C@@H]4CC[C@@]3(CCN12)O4 ; O=C1c2c(O)c(=O)ccn2N([C@H]2c3ccccc3SCc3cccc(F)c32)[C@@H]2[C@H]3C[C@@H]4CC[C@@]3(CCN12)O4]

### Step 1: (2S,4aS,14aR,14bR)-9-(benzyloxy)-14-(10-fluoro-6,11-dihydrodibenzo[b,e]thiepin-11-yl)-1,3,4,5,6,14,14a,14b-octahydro-2H-2,4a-epoxypyrido[1′,2′:1,6][1,2,4]triazino[3,4-a]isoquinoline-8,10-dione

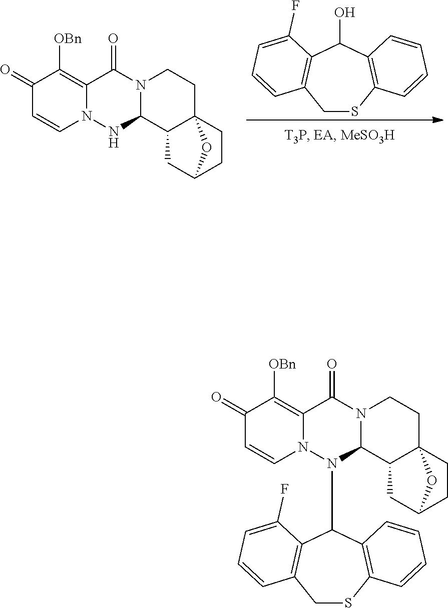

To a stirred solution of (2S,4aS,14aR,14bR)-9-(benzyloxy)-1,3,4,5,6,14,14a,14b-octahydro-2H-2,4a-epoxypyrido[1′,2′:1,6][1,2,4]triazino[3,4-a]isoquinoline-8,10-dione (80.0 mg, 0.20 mmol, 1.0 equiv.) and 10-fluoro-6,11-dihydrodibenzo[b,e]thiepin-11-ol (49.3 mg, 0.20 mmol, 1.0 equiv.) in T3P (1.0 mL) and EtOAc (1.0 mL) was added methanesulfonic acid (19.2 mg, 0.20 mmol, 1.0 equiv.) at room temperature under N₂ atmosphere. The resulting mixture was stirred for 16 h at 70° C. under N₂ atmosphere and then quenched with H₂O. The resulting mixture was extracted with EtOAc. The combined organic layers were dried over anhydrous Na₂SO₄. After filtration, the filtrate was concentrated under reduced pressure to give the crude title product (140 mg, crude). It was used in the next step directly without further purification. MS (ES, m/z): [M+1]⁺=622.3.

### Step 2: (2S,4aS,14aR,14bR)-14-((S)-10-fluoro-6,11-dihydrodibenzo[b,e]thiepin-11-yl)-9-hydroxy-1,3,4,5,6,14,14a,14b-octahydro-2H-2,4a-epoxypyrido[1′,2′: 1,6][1,2,4]triazino[3,4-a]isoquinoline-8,10-dione[3] and (2S,4aS,14aR,14bR)-14-((R)-10-fluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[4]

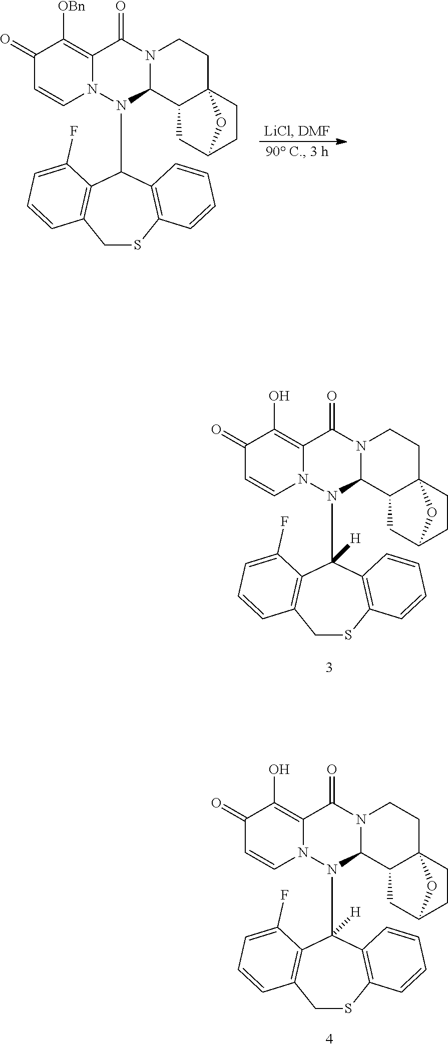

A stirred mixture of (2S,4aS,14aR,14bR)-9-(benzyloxy)-14-(10-fluoro-6,11-dihydrodibenzo[b,e]thiepin-11-yl)-1,3,4,5,6,14,14a, 14b-octahydro-2H-2,4a-epoxypyrido[1′,2′:1,6][1,2,4]triazino[3,4-a]isoquinoline-8,10-dione (140 mg, crude, 0.23 mmol, 1.0 equiv.) and LiCl (42.4 mg, 1.0 mmol, 5.0 equiv.) in DMA (1.0 mL) was stirred at 90° C. for 3 h. The crude product was purified by Prep-HPLC to afford the title product [3] (10 mg) a light yellow solid and [4] (15 mg) as a light yellow solid. Compound [3]: MS (ES, m/z): [M+1]⁺=532.2. Compound [4]: MS (ES, m/z): [M+1]⁺=532.2.

### Example 5 and 6

### Synthesis of (2S,4aS,14aR,14bR)-14-((S)-8,9-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[5] and (2S,4aS,14aR,14bR)-14-((R)-8,9-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[6]

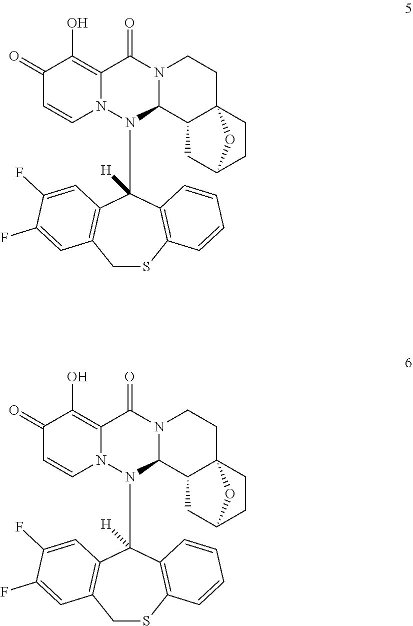 [SMILES: O=C1c2c(O)c(=O)ccn2N([C@H]2c3cc(F)c(F)cc3CSc3ccccc32)[C@@H]2[C@H]3C[C@@H]4CC[C@@]3(CCN12)O4 ; O=C1c2c(O)c(=O)ccn2N([C@@H]2c3cc(F)c(F)cc3CSc3ccccc32)[C@@H]2[C@H]3C[C@@H]4CC[C@@]3(CCN12)O4]

### Step 1: (2S,4aS,14aR,14bR)-9-(benzyloxy)-14-(8,9-difluoro-6,11-dihydrodibenzo[b,e]thiepin-11-yl)-1,3,4,5,6,14,14a, 14b-octahydro-2H-2,4a-epoxypyrido[1′,2′:1,6][1,2,4]triazino[3,4-a]isoquinoline-8,10-dione

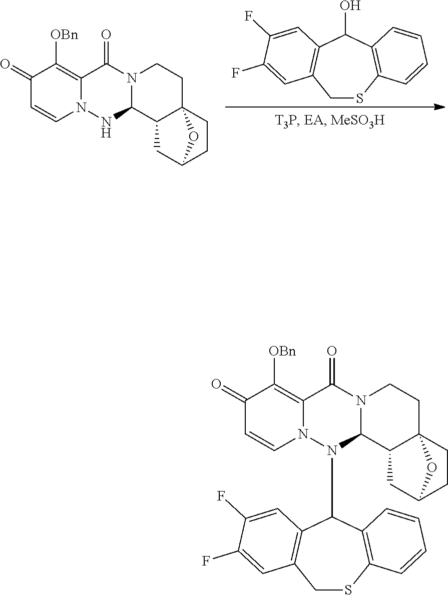

To a stirred solution of (2S,4aS,14aR,14bR)-9-(benzyloxy)-1,3,4,5,6,14,14a,14b-octahydro-2H-2,4a-epoxypyrido[1′,2′:1,6][1,2,4]triazino[3,4-a]isoquinoline-8,10-dione (80 mg, 0.20 mmol, 1 equiv.) and 8,9-difluoro-6,11-dihydrodibenzo[b,e]thiepin-11-ol (52.9 mg, 0.20 mmol, 1.0 equiv.) in T3P (1.0 mL) and EtOAc (1.0 mL) was added methanesulfonic acid (19.2 mg, 0.2 mmol, 1.0 equiv.) at room temperature under N₂ atmosphere. The resulting mixture was stirred for 16 h at 70° C. under N₂ atmosphere and then quenched with H₂O. The resulting mixture was extracted with EtOAc. The combined organic layers were dried over anhydrous Na₂SO₄. After filtration, the filtrate was concentrated under reduced pressure to give the title product (150 mg, crude). It was used in the next step directly without further purification. MS (ES, m/z): [M+1]⁺=640.3.

### Step 2: (2S,4aS,14aR,14bR)-14-((S)-8,9-difluoro-6,11-dihydrodibenzo[b,e]thiepin-1 l-yl)-9-hydroxy-1,3,4,5,6,14,14a,14b-octahydro-2H-2,4a-epoxypyrido[1′,2′: 1,6][1,2,4]triazino[3,4-a]isoquinoline-8,10-dione 151 and (2S,4aS,14aR,14bR)-14-((R)-8,9-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[6]

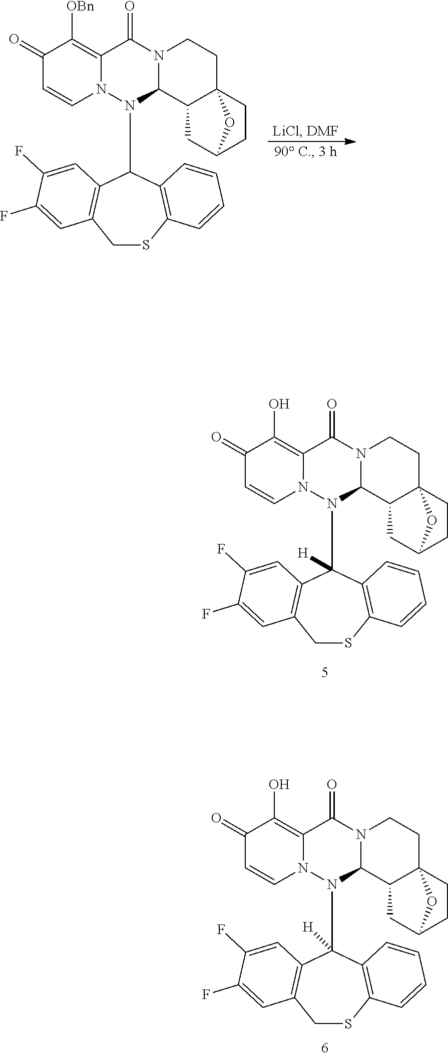

A mixture of (2S,4aS,14aR,14bR)-9-(benzyloxy)-14-(8,9-difluoro-6,11-dihydrodibenzo[b,e]thiepin-11-yl)-1,3,4,5,6,14,14a, 14b-octahydro-2H-2,4a-epoxypyrido[1′,2′:1,6][1,2,4]triazino[3,4-a]isoquinoline-8,10-dione (150 mg, crude, 0.23 mmol, 1.0 equiv.) and LiCl (42.4 mg, 1.0 mmol, 5.0 equiv.) in DMA (1.0 mL) was stirred at 90° C. for 3 h. The reaction solution was purified by Prep-HPLC to afford (2S,4aS,14aR,14bR)-14-((S)-8,9-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione 151 and (2S,4aS,14aR,14bR)-14-((R)-8,9-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[6] as a light yellow solid. Compound [5]: MS (ES, m/z): [M+1]⁺=550.2. Compound [6]: MS (ES, m/z): [M+1]⁺=550.2.

### Example 7 and 8

### Synthesis of (2S,4aS,14aR,14bR)-14-((S)-4,10-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[7] and (2S,4aS,14aR,14bR)-14-((R)-4,10-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[8]

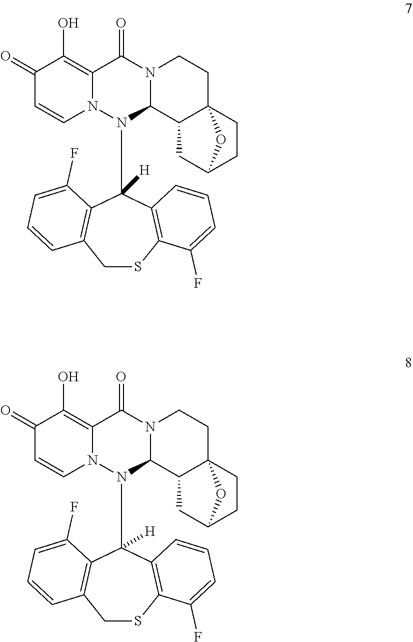 [SMILES: O=C1c2c(O)c(=O)ccn2N([C@@H]2c3cccc(F)c3SCc3cccc(F)c32)[C@@H]2[C@H]3C[C@@H]4CC[C@@]3(CCN12)O4 ; O=C1c2c(O)c(=O)ccn2N([C@H]2c3cccc(F)c3SCc3cccc(F)c32)[C@@H]2[C@H]3C[C@@H]4CC[C@@]3(CCN12)O4]

### Step 1: (2S,4aS,14aR,14bR)-9-(benzyloxy)-14-(4,10-difluoro-6,11-dihydrodibenzo[b,e]thiepin-11-yl)-1,3,4,5,6,14,14a,14b-octahydro-2H-2,4a-epoxypyrido[1′,2′:1,6][1,2,4]triazino[3,4-a]isoquinoline-8,10-dione

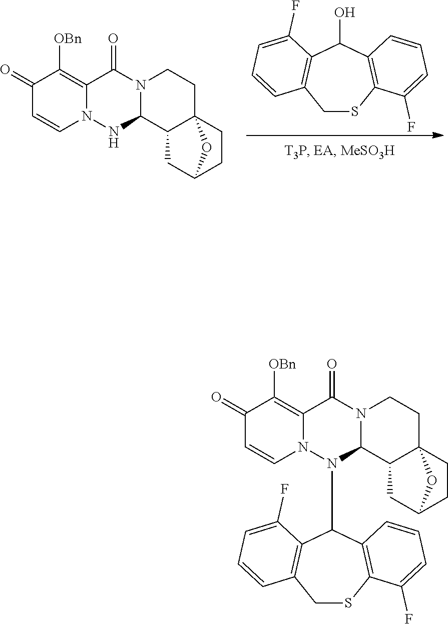

To a stirred solution of (2S,4aS,14aR,14bR)-9-(benzyloxy)-1,3,4,5,6,14,14a,14b-octahydro-2H-2,4a-epoxypyrido[1′,2′:1,6][1,2,4]triazino[3,4-a]isoquinoline-8,10-dione (80 mg, 0.20 mmol, 1.0 equiv.) and 4,10-difluoro-6,11-dihydrodibenzo[b,e]thiepin-11-ol (52.9 mg, 0.20 mmol, 1.0 equiv.) in T3P (1.0 mL) and EA (1.0 mL) was added methanesulfonic acid (19.2 mg, 0.20 mmol, 1 equiv.) at room temperature under N₂ atmosphere. The resulting mixture was stirred for 16 h at 70° C. under N₂ atmosphere and then quenched with H₂O. The resulting mixture was extracted with EtOAc. The combined organic layers were dried over anhydrous Na₂SO₄. After filtration, the filtrate was concentrated under reduced pressure to give the title product (140 mg, crude). It was used in the next step directly without further purification. MS (ES, m/z): [M+1]⁺=640.2.

### Step 2: (2S,4aS,14aR,14bR)-14-((S)-4,10-difluoro-6,11-dihydrodibenzo[b,e]thiepin-11-yl)-9-hydroxy-1,3,4,5,6,14,14a,14b-octahydro-2H-2,4a-epoxypyrido[1′,2′: 1,6][1,2,4]triazino[3,4-a]isoquinoline-8,10-dione[7] and (2S,4aS,14aR,14bR)-14-((R)-4,10-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione[8]

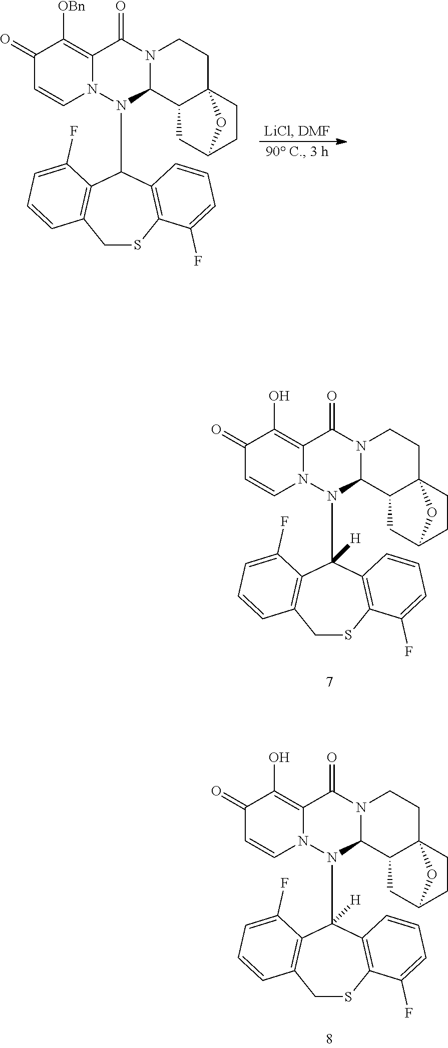

A stirred mixture of (2S,4aS,14aR,14bR)-9-(benzyloxy)-14-(4,10-difluoro-6,11-dihydrodibenzo[b,e]thiepin-1 l-yl)-1,3,4,5,6,14,14a,14b-octahydro-2H-2,4a-epoxypyrido[1′,2′:1,6][1,2,4]triazino[3,4-a]isoquinoline-8,10-dione (140 mg, crude, 0.22 mmol, 1.0 equiv.) and LiCl (42.4 mg, 1.0 mmol, 10.0 equiv.) in DMA (1.0 mL) was stirred at 90° C. for 3 h. The reaction solution was purified by Prep-HPLC to afford (2S,4aS,14aR,14bR)-14-((S)-4,10-difluoro-6,11-dihydrodibenzo[b,e]thiepin-11-yl)-9-hydroxy-1,3,4,5,6,14,14a,14b-octahydro-2H-2,4a-epoxypyrido[1′,2′: 1,6][1,2,4]triazino[3,4-a]isoquinoline-8,10-dione[7] and (2S,4aS,14aR,14bR)-14-((R)-4,10-difluoro-6,11-dihydrodibenzo[b,e]thiepin-11-yl)-9-hydroxy-1,3,4,5,6,14,14a,14b-octahydro-2H-2,4a-epoxypyrido[1′,2′:1,6][1,2,4]-triazino[3,4-a]isoquinoline-8,10-dione[8] as a light yellow solid. Compound [7]: MS (ES, m/z): [M+1]⁺=550.2. Compound [8]: MS (ES, m/z): [M+1]⁺=550.2.

### Example 9

### Synthesis of (2S,4aS,14aR,14bR)-14-(1,9-difluoro-10,11-dihydro-5H-dibenzo[a,d][7]annulen-5-yl)-9-hydroxy-1,3,4,5,6,14,14a,14b-octahydro-2H-2,4a-epoxypyrido[1′,2′:1,6][1,2,4]triazino[3,4-a]isoquinoline-8,10-dione

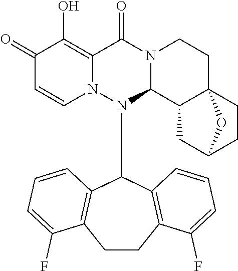 [SMILES: O=C1c2c(O)c(=O)ccn2N(C2c3cccc(F)c3CCc3c(F)cccc32)[C@@H]2[C@H]3C[C@@H]4CC[C@@]3(CCN12)O4]

To a stirred mixture of 1,9-difluoro-10,11-dihydro-5H-dibenzo[a,d][7]annulen-5-ol (50 mg, 0.20 mmol, 1.00 equiv.) and (2S,4aS,14aR,14bR)-9-(benzyloxy)-1,3,4,5,6,14,14a,14b-octahydro-2H-2,4a-epoxypyrido[1′,2′:1,6][1,2,4]triazino[3,4-a]isoquinoline-8,10-dione (80 mg, 0.20 mmol, 1.0 equiv.) in EtOAc (1.0 mL) and T3P in EtOAc (1.0 mL) were added methanesulfonic acid (20 mg, 0.20 mmol, 1.00 equiv.) in one portion at room temperature under N₂ atmosphere. After stirred at 80° C. for 16 h, the reaction mixture was cooled at rt and quenched with H₂O. The mixture was then extracted with EtOAc. The combined organic layers were washed with water and brine, dried over anhydrous Na₂SO₄ and concentrated. The residue was dissolved in DMA (1.0 mL) and LiCl (54 mg, 1.29 mmol, 10.0 equiv.) was added. The resulting mixture was stirred for 16 h at 90° C. under N₂ atmosphere. After cooled at rt, the crude material was purified by Prep-HPLC to afford the title compound (13 mg, 12%). MS (ES, m/z): [M+H]⁺=532.3.

### Example 10

### Synthesis of (2S,4aS,14aR,14bR)-14-(2,8-difluoro-10,11-dihydro-5H-dibenzo[a,d][7]-annulen-5-yl)-9-hydroxy-1,3,4,5,6,14,14a, 14b-octahydro-2H-2,4a-epoxypyrido-[1′,2′:1,6][1,2,4]triazino[3,4-a]isoquinoline-8,10-dione

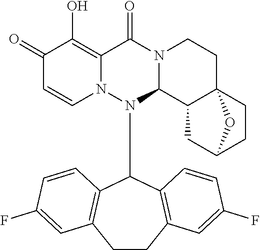 [SMILES: O=C1c2c(O)c(=O)ccn2N(C2c3ccc(F)cc3CCc3cc(F)ccc32)[C@@H]2[C@H]3C[C@@H]4CC[C@@]3(CCN12)O4]

Compound 10 was synthesized by proceeding analogously as described in Example 9 using 2,8-difluoro-10,11-dihydro-5H-dibenzo[a,d][7]annulen-5-ol instead of 1,9-difluoro-10,11-dihydro-5H-dibenzo[a,d][7]annulen-5-ol. MS (ES, m/z): [M+H]⁺=532.3.

### Example 11

### Synthesis of (14aS)-6-(7,8-difluoro-6,11-dihydrodibenzo[b,e]thiepin-1 l-yl)-11-hydroxy-5a,6,14,14a-tetrahydro-1H,5H-pyrido[2,1-f]pyrrolo[1′,2′:4,5]pyrazino[2,1-c][1,2,4]triazine-3,10,12(2H)-trione

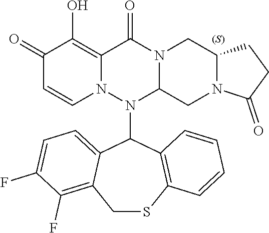

### Step 1: 2-[[(2S)-5-oxopyrrolidin-2-yl]methyl]-dihydroisoindole-1,3-dione

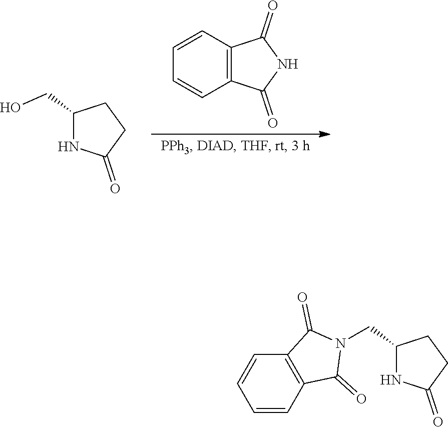

To a stirred solution of (5S)-5-(hydroxymethyl)pyrrolidin-2-one (10.00 g, 86.8 mmol, 1.0 equiv.), phthalimide (14.06 g, 95.5 mmol, 1.1 equiv.) and PPh₃ (25.06 g, 95.5 mmol, 1.1 equiv.) in THE (150 mL) was added DIAD (19.3 g, 95.5 mmol, 1.1 equiv.) dropwise at room temperature under nitrogen atmosphere. The resulting mixture was stirred for 3 h. The precipitated solids were collected by filtration and washed with EtOAc to afford the title compound (13.4 g, 63%) as an off-white solid. MS (ES, m/z): [M+1]⁺=245.1.

### Step 2: 2-[[(2S)-5-oxo-1-(prop-2-en-1-yl)pyrrolidin-2-yl]methyl]isoindole-1,3-dione

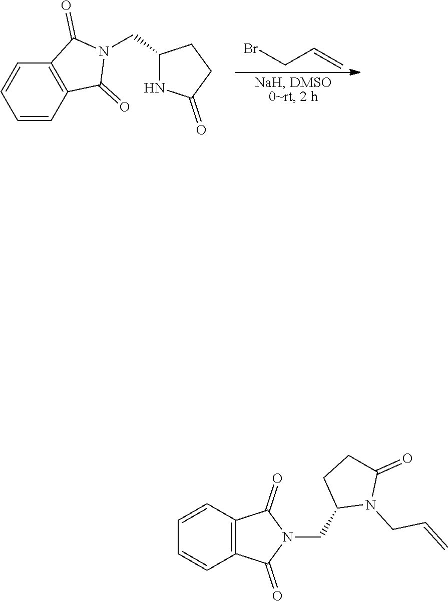

To a stirred solution of 2-[[(2S)-5-oxopyrrolidin-2-yl]methyl]isoindole-1,3-dione (13.4 g, 54.8 mmol, 1.0 equiv.) in DMSO (300 mL) was added NaH (2.63 g, 65.7 mmol, 1.2 equiv., 60% in mineral oil) in portions at room temperature under nitrogen atmosphere. The resulting mixture was stirred for 0.5 h at room temperature. To the above mixture was added allyl bromide (13.27 g, 109.7 mmol, 2.0 equiv.) dropwise over 10 min at room temperature. The resulting mixture was stirred for additional 2 h at room temperature. The resulting mixture was diluted with EtOAc and then quenched with aq. HCl solution (1.0 M) at 0° C. The resulting mixture was extracted with EtOAc and the combined organic layers were washed with water and brine, dried over anhydrous Na₂SO₄. After filtration, the filtrate was concentrated under reduced pressure. The residue was purified by silica gel column chromatography, eluted with PE/EtOAc (1:1), to afford the title compound (13.5 g, 86.5%) as light yellow oil. MS (ES, m/z): [M+1]⁺=285.1.

### Step 3: 2-[(2S)-2-[(1,3-dioxo-3a,7a-dihydroisoindol-2-yl)methyl]-5-oxopyrrolidin-1-yl]acetaldehyde

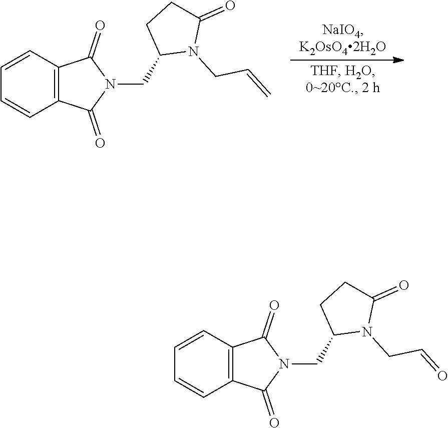 [SMILES: C=CCN1C(=O)CC[C@H]1CN1C(=O)c2ccccc2C1=O ; O=CCN1C(=O)CC[C@H]1CN1C(=O)c2ccccc2C1=O]

To a stirred solution of 2-[[(2S)-5-oxo-1-(prop-2-en-1-yl)pyrrolidin-2-yl]methyl]-3a,7a-dihydroisoindole-1,3-dione (6.00 g, 20.1 mmol, 1.0 equiv) in THE (120 mL) and H₂O (30 mL) were added K₂OSO₄.2H₂O (1.54 g, 4.2 mmol, 0.20 equiv.) and NaIO₄ (22.4 g, 104.7 mmol, 5.0 equiv.) in portions at 0° C. The resulting mixture was stirred for 2 h at room temperature and then filtered. The filter cake was washed with DCM and the filtrate was extracted with DCM. The combined organic layers were washed with brine, dried over anhydrous Na₂SO₄. After filtration, the filtrate was concentrated under reduced pressure to afford the title compound (4.1 g, 67%) as a light brown semi-solid. MS (ES, m/z): [M+1]⁺=287.1.

### Step 4: Synthesis of 2-[[(2S)-1-(2,2-dimethoxyethyl)-5-oxopyrrolidin-2-yl]methyl]-dihydroisoindole-1,3-dione

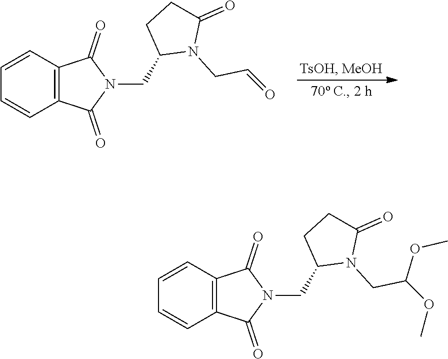 [SMILES: O=CCN1C(=O)CC[C@H]1CN1C(=O)c2ccccc2C1=O ; COC(CN1C(=O)CC[C@H]1CN1C(=O)c2ccccc2C1=O)OC]

A solution of 2-[(2S)-2-[(1,3-dioxo-3a,7a-dihydroisoindol-2-yl)methyl]-5-oxopyrrolidin-1-yl]acetaldehyde (4.1 g, 14.2 mmol, 1.0 equiv.) and TsOH (245 mg, 1.4 mmol, 0.1 equiv.) in MeOH (100 mL) was stirred for 2 h at 70° C. under nitrogen atmosphere. The mixture was allowed to cool to room temperature and then concentrated under reduced pressure. The residue was dissolved in DMF and then purified by reverse flash chromatography to afford the title compound (3.1 g, 65%) as light brown oil. MS (ES, m/z): [M+Na]⁺=355.2.

### Step 5: (5S)-5-(aminomethyl)-1-(2,2-dimethoxyethyl)pyrrolidin-2-one

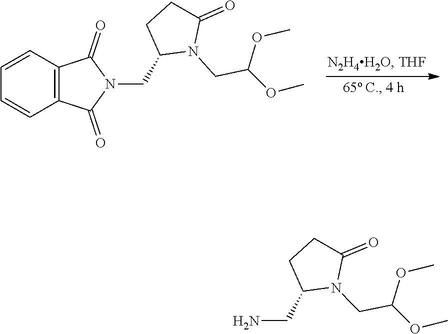 [SMILES: COC(CN1C(=O)CC[C@H]1CN1C(=O)c2ccccc2C1=O)OC ; COC(CN1C(=O)CC[C@H]1CN)OC]

To a stirred solution of 2-[[(2S)-1-(2,2-dimethoxyethyl)-5-oxopyrrolidin-2-yl]methyl]-3a,7a-dihydroisoindole-1,3-dione (3.10 g, 9.3 mmol, 1.0 equiv.) in THF (60 mL) was added hydrazine (1.49 g, 29.8 mmol, 3.2 equiv.) at room temperature. The resulting mixture was stirred for 4 h at 65° C. The mixture was cooled at rt and then filtered. The filter cake was washed with THF and the filtrate was concentrated under reduced pressure and dissolved in water. The resulting mixture was washed with EtOAc and the aqueous solution was freeze-dried to afford the title compound (1.5 g, 80%) as a light yellow oil. ¹HNMR (300 MHz, CDCl₃, ppm) δ 4.55 (dd, J=5.7, 5.0 Hz, 1H), 3.80-3.62 (m, 2H), 3.41 (s, 6H), 3.16-3.03 (m, 1H), 2.97-2.77 (m, 2H), 2.56-2.27 (m, 2H), 2.22-2.08 (m, 1H), 1.94-1.81 (m, 1H).

### Step 6: 3-(benzyloxy)-1-[(tert-butoxycarbonyl)amino]-4-oxopyridine-2-carboxylic acid

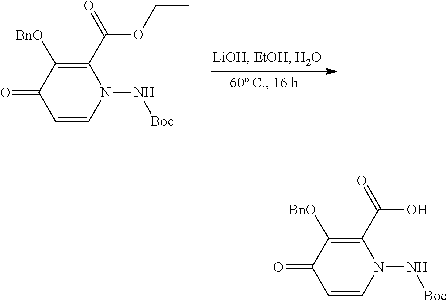 [SMILES: CCOC(=O)c1c(OCc2ccccc2)c(=O)ccn1NC(=O)OC(C)(C)C ; CC(C)(C)OC(=O)Nn1ccc(=O)c(OCc2ccccc2)c1C(=O)O]

To a stirred solution of ethyl 3-(benzyloxy)-1-[(tert-butoxycarbonyl)amino]-4-oxopyridine-2-carboxylate (3.50 g, 9.0 mmol, 1.0 equiv.) in EtOH (40 mL) and H₂O (10 mL) was added LiOH (0.86 g, 35.9 mmol, 4.0 equiv.) at room temperature. The resulting mixture was stirred for 16 h at 60° C. and then concentrated under reduced pressure. The mixture was neutralized to pH 6 with HOAc and the resulting mixture was purified by Prep-HPLC to afford the title compound (1.8 g, 55%) as off-white solid. MS (ES, m/z): [M+1]⁺=361.2.

### Step 7: 3-(benzyloxy)-1-[(tert-butoxycarbonyl)amino]-N-[[(2S)-1-(2,2-dimethoxyethyl)-5-oxopyrrolidin-2-yl]methyl]-4-oxopyridine-2-carboxamide

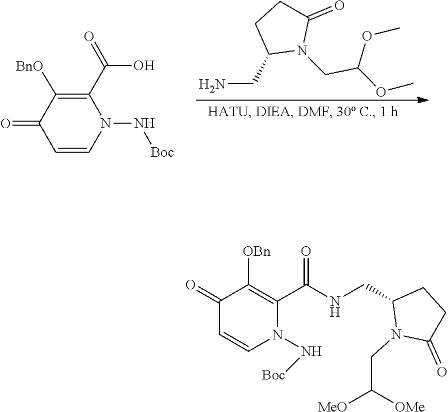 [SMILES: CC(C)(C)OC(=O)Nn1ccc(=O)c(OCc2ccccc2)c1C(=O)O ; COC(CN1C(=O)CC[C@H]1CN)OC ; COC(CN1C(=O)CC[C@H]1CNC(=O)c1c(OCc2ccccc2)c(=O)ccn1NC(=O)OC(C)(C)C)OC]

To a stirred solution of 3-(benzyloxy)-1-[(tert-butoxycarbonyl)amino]-4-oxopyridine-2-carboxylic acid (700 mg, 1.9 mmol, 1.0 equiv.) and HATU (1.1 g, 2.9 mmol, 1.5 equiv.) in DMF (10 mL) were added (5S)-5-(aminomethyl)-1-(2,2-dimethoxyethyl)-pyrrolidin-2-one (785 mg, 3.9 mmol, 2.0 equiv.) and DIEA (627 mg, 4.8 mmol, 2.5 equiv.). The resulting mixture was stirred for 2 h at room temperature. The reaction solution was purified by reverse flash chromatography to give (550 mg, 52%) of the title compound as an off-white solid. MS (ES, m/z): [M+1]⁺=545.3.

### Step 8: (14aS)-11-(benzyloxy)-5a,6,14,14a-tetrahydro-1H,5H-pyrido[2,1-f]pyrrolo-[1′,2′:4,5]pyrazino[2,1-c][1,2,4]triazine-3,10,12(2H)-trione

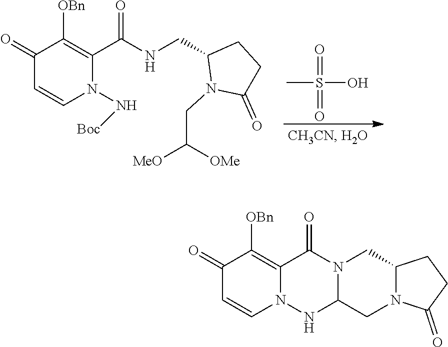

To a stirred solution of 3-(benzyloxy)-1-[(tert-butoxycarbonyl)amino]-N-[[(2S)-1-(2,2-dimethoxyethyl)-5-oxopyrrolidin-2-yl]methyl]-4-oxopyridine-2-carboxamide (550 mg, 1.0 mmol, 1.0 equiv.) in CH₃CN (9.0 mL) and H₂O (1.5 mL) was added methane sulfonic acid (291 mg, 3.0 mmol, 3.0 equiv.) dropwise at 60° C. under nitrogen atmosphere. The resulting mixture was stirred for 16 h at 60° C. under nitrogen atmosphere. The organic solvent was removed under reduced pressure and the resulting mixture was basified to pH 8 with saturated aq. Na₂CO₃ solution and then extracted with DCM. The combined organic layers were washed with brine, dried over anhydrous Na₂SO₄. After filtration, the filtrate was concentrated under reduced pressure. The residue was purified by silica gel column chromatography, eluted with DCM/MeOH (10:1) to afford the title compound (200 mg, 52%) as a light yellow solid. MS (ES, m/z): [M+1]⁺=381.1.

### Step 9: (14aS)-11-(benzyloxy)-6-(7,8-difluoro-6,11-dihydrodibenzo[b,e]thiepin-11-yl)-5a,6,14,14a-tetrahydro-1H,5H-pyrido[2,14]pyrrolo[1′,2′:4,5]pyrazino[2,1-c][1,2,4]triazine-3,10,12(2H)-trione

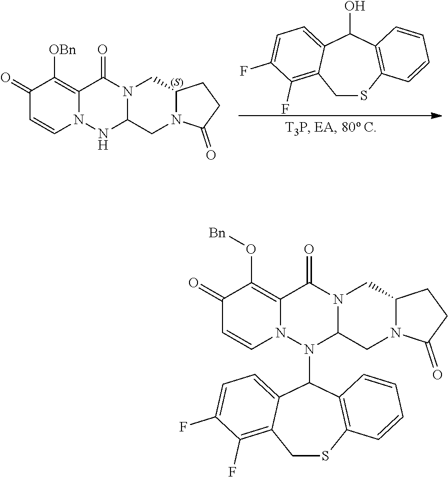

A mixture of (14aS)-11-(benzyloxy)-5a,6,14,14a-tetrahydro-1H,5H-pyrido[2,1-f]pyrrolo[1′,2′:4,5]pyrazino[2,1-c][1,2,4]triazine-3,10,12(2H)-trione (100 mg, 0.26 mmol, 1.0 equiv.) and 7,8-difluoro-6,11-dihydrodibenzo[b,e]thiepin-11-ol (77 mg, 0.2 mmol, 1.1 equiv.) in T3P (1.0 mL) and EtOAc (0.5 mL) was stirred for 16 h at 80° C. under nitrogen atmosphere. The resulting mixture was diluted with water and extracted with EtOAc. The combined organic layers were washed with brine and dried over anhydrous Na₂SO₄. After filtration, the filtrate was concentrated under reduced pressure to afford the title compound (150 mg, crude) as a light yellow solid. The crude product was used in next step directly without further purification. MS (ES, m/z): [M+1]⁺=627.1.

### Step 10: (14aS)-6-(7,8-difluoro-6,11-dihydrodibenzo[b,e]thiepin-11-yl)-11-hydroxy-5a,6,14,14a-tetrahydro-1H,5H-pyrido[2,1-f]pyrrolo[1′,2′:4,5]pyrazino[2,1-c][1,2,4]triazine-3,10,12(2H)-trione

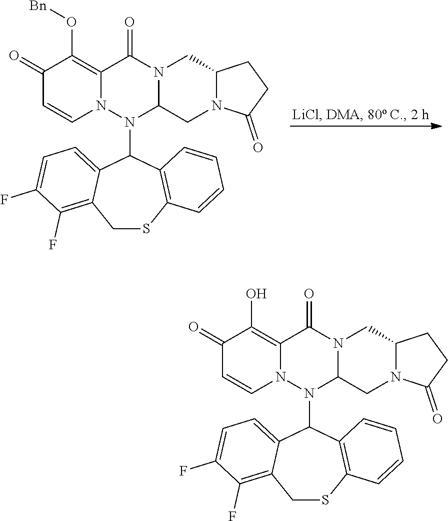

To a stirred solution of (14aS)-11-(benzyloxy)-6-(7,8-difluoro-6,11-dihydrodibenzo-[b,e]thiepin-11-yl)-5a,6,14,14a-tetrahydro-1H,5H-pyrido[2,1-f]pyrrolo[1′,2′:4,5]pyrazino[2,1-c][1,2,4]triazine-3,10,12(2H)-trione (150 mg, crude, 0.24 mmol, 1.0 equiv.) in DMA (2.0 mL) was added LiCl (112 mg, 2.6 mmol, 10.0 equiv.). The resulting mixture was stirred for 2 h at 80° C. and then purified by Prep-HPLC to afford the title compound (100 mg, 2 steps 71%) as a white solid. MS (ES, m/z): [M+1]⁺=537.1.

100 mg of the title compound was separated by Chiral-Prep-HPLC to afford four isomers, 11a, 11b, 11c and 11d.

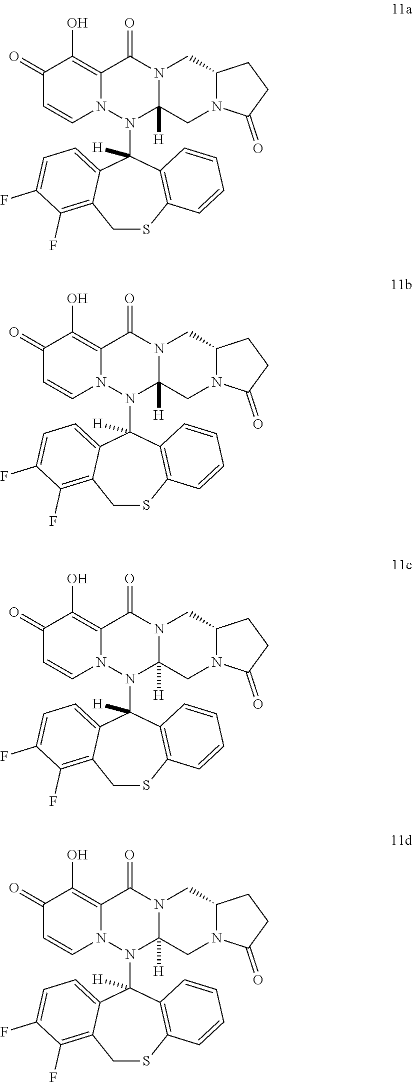 [SMILES: O=C1CC[C@H]2CN3C(=O)c4c(O)c(=O)ccn4N([C@@H]4c5ccccc5SCc5c4ccc(F)c5F)[C@H]3CN12 ; O=C1CC[C@H]2CN3C(=O)c4c(O)c(=O)ccn4N([C@H]4c5ccccc5SCc5c4ccc(F)c5F)[C@H]3CN12 ; O=C1CC[C@H]2CN3C(=O)c4c(O)c(=O)ccn4N([C@@H]4c5ccccc5SCc5c4ccc(F)c5F)[C@@H]3CN12 ; O=C1CC[C@H]2CN3C(=O)c4c(O)c(=O)ccn4N([C@H]4c5ccccc5SCc5c4ccc(F)c5F)[C@@H]3CN12]

First-eluting isomer: tR 0.83 min, (4.6 mg, 4.6%), MS (ES, m/z): [M+1]⁺=537.2 second-eluting isomer: tR 1.06 min, (8.5 mg, 8.50%), MS (ES, m/z): [M+1]⁺=537.1, third-eluting isomer: tR 1.28 min, (36 mg, 36.0%), MS (ES, m/z): [M+1]⁺=537.1. and fourth-eluting isomer: tR 1.69 min, (33 mg, 33.0%), MS (ES, m/z): [M+1]⁺=537.1.

### Example 12

### Synthesis of (14aR)-6-(7,8-difluoro-6,11-dihydrodibenzo[b,e]thiepin-11-yl)-11-hydroxy-5a,6,14,14a-tetrahydro-1H,5H-pyrido[2,1-f]pyrrolo[1′,2′:4,5]pyrazino[2,1-c][1,2,4]triazine-3,10,12(2H)-trione

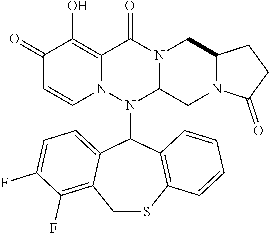

Compound 12 was synthesized by proceeding analogously as described in Example 11 above, but using (5R)-5-(hydroxymethyl)pyrrolidin-2-one instead of (5S)-5-(hydroxymethyl)-pyrrolidin-2-one in step 1. MS (ES, m/z): [M+1]⁺=537.1.

### Example 13

### Synthesis of rac-(R)-14-((S)-7,8-difluoro-6,11-dihydrodibenzo[b,e]thiepin-11-yl)-9-hydroxy-5,6,14,14a-tetrahydro-[1,2,3]triazolo[5′,1′:3,4]pyrazino[2,1-c]pyrido[2,1-f][1,2,4]triazine-8,10-dione and rac-(R)-14-((R)-7,8-difluoro-6,11-dihydrodibenzo[b,e]thiepin-11-yl)-9-hydroxy-5,6,14,14a-tetrahydro-[1,2,3]triazolo[5′,1′:3,4]pyrazino[2,1-c]pyrido[2,1-f][1,2,4]triazine-8,10-dione[rac-13a and rac-13b]

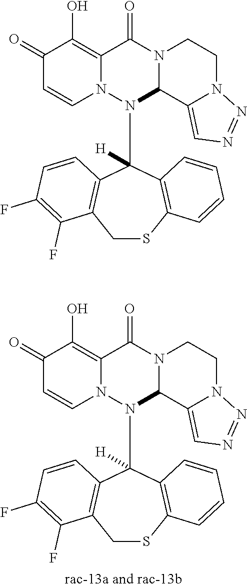 [SMILES: O=C1c2c(O)c(=O)ccn2N([C@@H]2c3ccccc3SCc3c2ccc(F)c3F)[C@@H]2c3cnnn3CCN12 ; O=C1c2c(O)c(=O)ccn2N([C@H]2c3ccccc3SCc3c2ccc(F)c3F)[C@@H]2c3cnnn3CCN12]

### Step 1: 2-(2-azidoethyl)isoindoline-1,3-dione

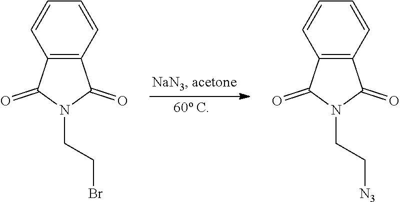 [SMILES: O=C1c2ccccc2C(=O)N1CCBr ; [N-]=[N+]=NCCN1C(=O)c2ccccc2C1=O]

A 50-mL round-bottom flask was charged with N-(2-bromoethyl)phthalimide (2.0 g, 7.87 mmol, 1.00 equiv.), acetone (20.0 mL), H₂O (10.0 mg) and sodium azide (1.54 g, 23.7 mmol, 3.0 equiv.). The reaction mixture was then stirred for 24 h at 60° C. After cooling to rt, the mixture was filtered and the filtrate was concentrated under vacuum. The residue was purified by silica gel column chromatography (ethyl acetate/petroleum ether 1/2), to afford the title compound (1.5 g, 88%). MS (ES, m/z): [M+1]⁺=245.1.

### Step 2: 2-(2-(5-(diethoxymethyl)-1H-1,2,3-triazol-1-yl)ethyl)isoindoline-1,3-dione

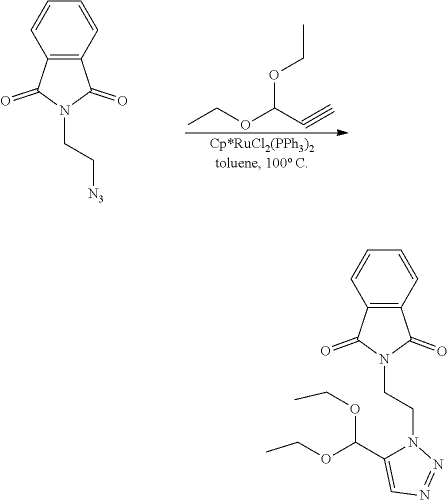

A 100-mL round-bottom flask was charged with 2-(2-azidoethyl)isoindole-1,3-dione (3.00 g, 13.8 mmol, 1.0 equiv.), 3,3-diethoxypropyne (3.56 g, 27.7 mmol, 2.0 equiv.), Cp*RuCl(PPh₃)₂ (552 mg, 0.69 mmol, 0.05 equiv.) and toluene (30 mL). The resulting solution was stirred for 6 h at 100° C. After cooling to rt, the reaction mixture was concentrated under vacuum and the residue was purified by silica gel column (ethyl acetate/petroleum ether 1/1) to afford 3.15 g (66%) of the title compound. MS (ES, m/z): [M+1]⁺=345.1.

### Step 3: 2-(5-(diethoxymethyl)-1H-1,2,3-triazol-1-yl)ethan-1-amine

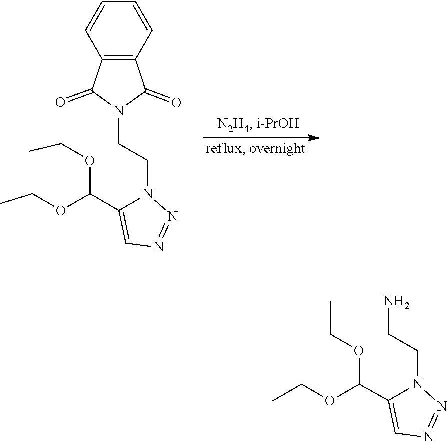 [SMILES: CCOC(OCC)c1cnnn1CCN1C(=O)c2ccccc2C1=O ; CCOC(OCC)c1cnnn1CCN]

A 50-mL round-bottom flask was charged with 2-[2-[5-(diethoxymethyl)-1,2,3-triazol-1-yl]ethyl]isoindole-1,3-dione (680 mg, 1.9 mmol, 1.0 equiv), i-PrOH (10 mL) and NH₂NH₂.H₂O (988 mg, 19.8 mmol, 10 equiv.). The reaction mixture was then stirred for 16 h at 80° C. After cooling to rt, the mixture was filtered and the filtrate was concentrated under vacuum to give 320 mg (yield 75%) of the title compound. MS (ES, m/z): [M+1]⁺=215.1.

### Step 4: tert-butyl (3-(benzyloxy)-2-((2-(5-(diethoxymethyl)-1H-1,2,3-triazol-1-yl)ethyl)carbamoyl)-4-oxopyridin-1(4H)-yl)carbamate

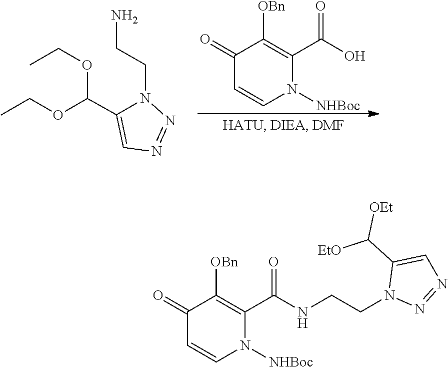 [SMILES: CCOC(OCC)c1cnnn1CCN ; CC(C)(C)OC(=O)Nn1ccc(=O)c(OCc2ccccc2)c1C(=O)O ; CCOC(OCC)c1cnnn1CCNC(=O)c1c(OCc2ccccc2)c(=O)ccn1NC(=O)OC(C)(C)C]

A solution of 2-[5-(diethoxymethyl)-1,2,3-triazol-1-yl]ethanamine (320 mg, 1.5 mmol, 1.0 equiv), 3-(benzyloxy)-1-[(tert-butoxycarbonyl)amino]-4-oxopyridine-2-carboxylic acid (538 mg, 1.5 mmol, 1.0 equiv.), HATU (681 mg, 1.8 mmol, 1.2 equiv.), and DIEA (386 mg, 3.0 mmol, 2.0 equiv) in DMF (2.0 mL, 0.027 mmol, 0.02 equiv.) was stirred for 16 h at room temperature. The resulting mixture was then diluted with water and extracted with ethyl acetate. The organic layer was dried over anhydrous sodium sulfate and concentrated under vacuum. The residue was purified by silica gel column chromatography (dichloromethane/methanol 20/1) to give 450 mg (54%) of the title compound. MS (ES, m/z): [M+1]⁺=557.3.

### Step 5: 9-(benzyloxy)-5,6,14,14a-tetrahydro-[1,2,3]triazolo[5′,1′:3,4]pyrazino[2,1-c]pyrido[2,1-f][1,2,4]triazine-8,10-dione

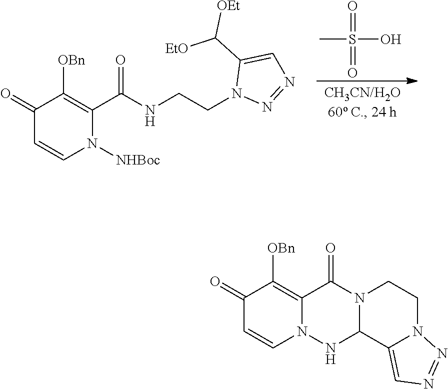

A solution of 3-(benzyloxy)-1-[(tert-butoxycarbonyl)amino]-N-[2-[5-(diethoxymethyl)-1,2,3-triazol-1-yl]ethyl]-4-oxopyridine-2-carboxamide (775 mg, 1.4 mmol, 1.0 equiv.), H₂O (2.5) and MeSO₃H (801 mg, 4.6 mmol, 3.3 equiv.) in CH₃CN (15 mL) was stirred for 24 h at 60° C. After cooling to rt, the reaction mixture was basified to pH=8 by adding TEA. The resulting mixture was concentrated and the residue was purified by silica gel column chromatography (dichloromethane/methanol, 10/1) to afford 1% mg (38%) of the title compound. MS (ES, m/z): [M+1]⁺=365.2.

### Step 6: rac-(R)-9-(benzyloxy)-14-((S)-7,8-difluoro-6,11-dihydrodibenzo[b,e]thiepin-11-yl)-5,6,14,14a-tetrahydro-[1,2,3]triazolo[5′,1′:3,4]pyrazino[2,1-c]pyrido[2,1-f][1,2,4]triazine-8,10-dione and rac-(R)-9-(benzyloxy)-14-((R)-7,8-difluoro-6,11-dihydrodibenzo[b,e]thiepin-11-yl)-5,6,14,14a-tetrahydro-[1,2,3]triazolo[5′,1′:3,4]pyrazino[2,1-c]pyrido[2,1-f][1,2,4]triazine-8,10-dione

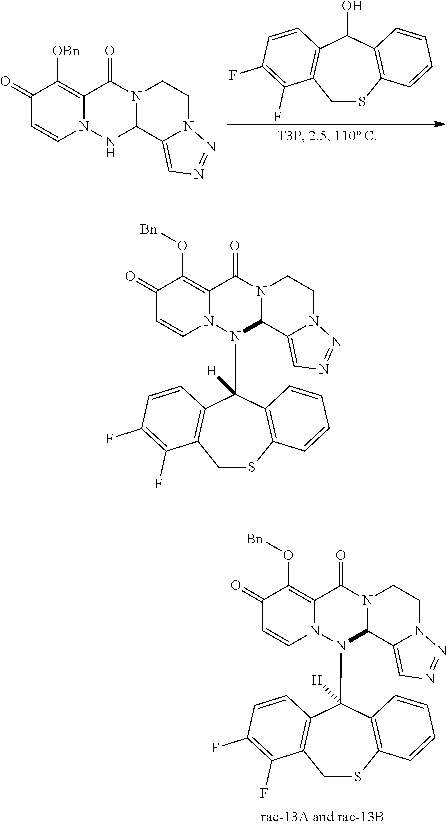 [SMILES: O=C1c2c(OCc3ccccc3)c(=O)ccn2NC2c3cnnn3CCN12 ; OC1c2ccccc2SCc2c1ccc(F)c2F ; O=C1c2c(OCc3ccccc3)c(=O)ccn2N([C@@H]2c3ccccc3SCc3c2ccc(F)c3F)[C@@H]2c3cnnn3CCN12 ; O=C1c2c(OCc3ccccc3)c(=O)ccn2N([C@H]2c3ccccc3SCc3c2ccc(F)c3F)[C@@H]2c3cnnn3CCN12]

A reaction vessel was charged with 9-(benzyloxy)-5,6,14,14a-tetrahydro-[1,2,3]-triazolo[5′,1′:3,4]pyrazino[2,1-c]pyrido[2,1-f][1,2,4]triazine-8,10-dione (100 mg, 0.27 mmol, 1.0 equiv.), 7,8-difluoro-6,11-dihydrodibenzo[b,e]thiepin-11-ol (72 mg, 0.27 mmol, 1.0 equiv.) and T3P in EtOAc solution (2.50 mL, T3P/EtOAc 2/1). The resulting solution was stirred for 2.5 h at 100° C. After cooling to rt, the reaction mixture was added water and then extracted with EtOAc. The organic layer was dried over NaSO₄ and concentrated. The residue was purified by silica gel column (dichloromethane/methanol 10/1) to give compound rac-13A and rac-13B. MS (ES, m/z): [M+1]⁺=611.1. LCMS condition: Column: Shim-pack XR-ODS, 50*3.0 mm, 2. uM; Mobile phase A: water/0.1% FA; Mobile phase B: CH₃CN/0.05% FA. One of rac-13A and rac-13B as tR 1.36 min and the other has tR 1.46 min.

### Step 7: rac-(R)-14-((S)-7,8-difluoro-6,11-dihydrodibenzo[b,e]thiepin-11-yl)-9-hydroxy-5,6,14,14a-tetrahydro-[1,2,3]triazolo[5′,1′:3,4]pyrazino[2,1-c]pyrido[2,1-f][1,2,4]triazine-8,10-dione and rac-(R)-14-((R)-7,8-difluoro-6,11-dihydrodibenzo[b,e]thiepin-1 l-yl)-9-hydroxy-5,6,14,14a-tetrahydro-[1,2,3]triazolo[5′,1′:3,4]pyrazino[2,1-c]pyrido[2,1-f][1,2,4]triazine-8,10-dione[rac-13a and rac-13b]

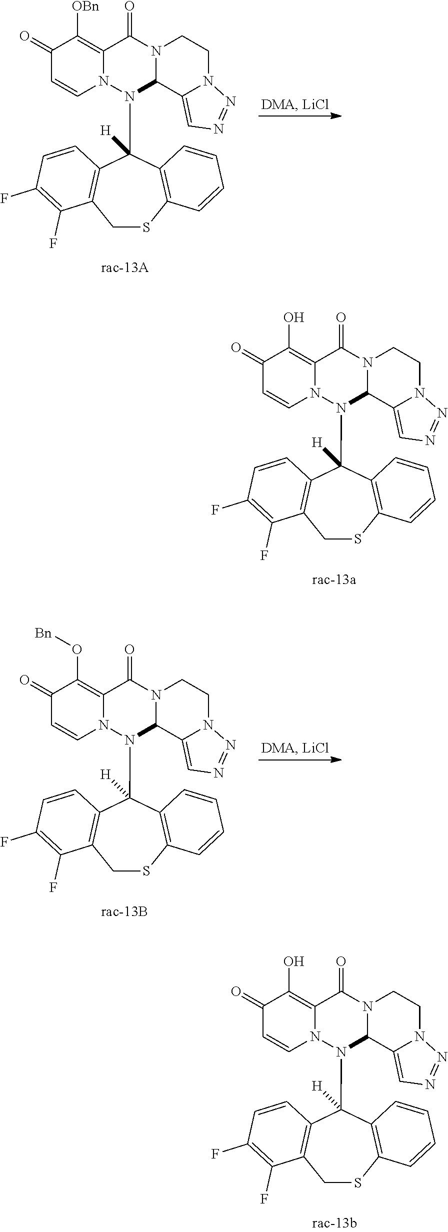 [SMILES: O=C1c2c(OCc3ccccc3)c(=O)ccn2N([C@@H]2c3ccccc3SCc3c2ccc(F)c3F)[C@@H]2c3cnnn3CCN12 ; O=C1c2c(O)c(=O)ccn2N([C@@H]2c3ccccc3SCc3c2ccc(F)c3F)[C@@H]2c3cnnn3CCN12 ; O=C1c2c(OCc3ccccc3)c(=O)ccn2N([C@H]2c3ccccc3SCc3c2ccc(F)c3F)[C@@H]2c3cnnn3CCN12 ; O=C1c2c(O)c(=O)ccn2N([C@H]2c3ccccc3SCc3c2ccc(F)c3F)[C@@H]2c3cnnn3CCN12]

A mixture of 9-(benzyloxy)-14-(7,8-difluoro-6,11-dihydrodibenzo[b,e]thiepin-1 l-yl)-5,6,14,14a-tetrahydro-[1,2,3]triazolo[5′,1′:3,4]pyrazino[2,1-c]pyrido[2,1-f][1,2,4]triazine-8,10-dione (rac-13A or rac-13B, 25.00 mg, 0.041 mmol, 1.00 equiv.), LiCl (8.68 mg, 0.205 mmol, 5.00 equiv.) and DMA (1.00 mL) was stirred for 1 h at 90° C. After cooling to rt, the reaction solution was purified by Prep-HPLC with afford 3.3 mg (15.4%) of compound rac-13a or rac-13b. MS (ES, m/z): [M+1]⁺=521.2.

A mixture of 9-(benzyloxy)-14-((S)-7,8-difluoro-6,11-dihydrodibenzo[b,e]thiepin-11-yl)-5,6,14,14a-tetrahydro-[1,2,3]triazolo[5′,1′:3,4]pyrazino[2,1-c]pyrido[2,1-f][1,2,4]triazine-8,10-dione (rac-13A or rac-13B, 17.00 mg, 0.028 mmol, 1.00 equiv), LiCl (5.90 mg, 0.139 mmol, 5.00 equiv) and DMA (1.00 mL) was stirred for 1 h at 90° C. The reaction solution was cooled and purified by Prep-HPLC to afford 2.3 mg (15.9%) of compound rac-13a or rac-13b as an off-white solid. MS (ES, m/z): [M+1]⁺=521.2.

### Example 14

### Synthesis of (((2S,4aS,14aR,14bR)-14-((S)-7,8-difluoro-6,11-dihydrodibenzo[b,e]thiepin-11-yl)-8,10-dioxo-1,3,4,5,6,8,10,14,14a,14b-decahydro-2H-2,4a-epoxypyrido[1′,2′:1,6][1,2,4]-triazino[3,4-a]isoquinolin-9-yl)oxy)methyl methyl carbonate or (((2S,4aS,14aR,14bR)-14-((R)-7,8-difluoro-6,11-dihydrodibenzo[b,e]thiepin-1 l-yl)-8,10-dioxo-1,3,4,5,6,8,10,14,14a,14b-decahydro-2H-2,4a-epoxypyrido[1′,2′:1,6][1,2,4]-triazino[3,4-a]isoquinolin-9-yl)oxy)methyl methyl carbonate

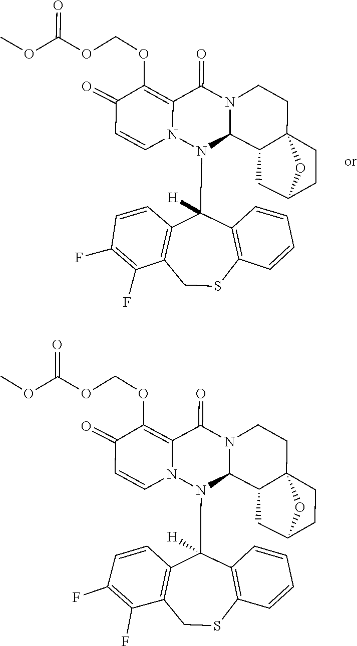 [SMILES: COC(=O)OCOc1c2n(ccc1=O)N([C@@H]1c3ccccc3SCc3c1ccc(F)c3F)[C@@H]1[C@H]3C[C@@H]4CC[C@@]3(CCN1C2=O)O4 ; COC(=O)OCOc1c2n(ccc1=O)N([C@H]1c3ccccc3SCc3c1ccc(F)c3F)[C@@H]1[C@H]3C[C@@H]4CC[C@@]3(CCN1C2=O)O4]

To a stirred mixture of (2S,4aS,14aR,14bR)-14-((S)-7,8-difluoro-6,11-dihydrodibenzo-[b,e]thiepin-11-yl)-9-hydroxy-1,3,4,5,6,14,14a,14b-octahydro-2H-2,4a-epoxypyrido[1′,2′:1,6]-[1,2,4]triazino[3,4-a]isoquinoline-8,10-dione or (2S,4aS,14aR,14bR)-14-((R)-7,8-difluoro-6,11-dihydrodibenzo-[b,e]thiepin-11-yl)-9-hydroxy-1,3,4,5,6,14,14a,14b-octahydro-2H-2,4a-epoxypyrido[1′,2′:1,6]-[1,2,4]triazino[3,4-a]isoquinoline-8,10-dione (10 mg, 0.018 mmol, 1.0 equiv.), K₂CO₃ (5.0 mg, 0.036 mmol, 2.0 equiv.) and KI (6.0 mg, 0.036 mmol, 2.0 equiv.) in DMA (0.20 mL) was added chloromethyl methyl carbonate (4.5 mg, 0.036 mmol, 2.0 equiv.) in portions at room temperature under N₂ atmosphere. The resulting mixture was stirred for 16 h at 80 degrees C. The crude product was purified by Prep-HPLC to afford the title compound (2.0 mg, 17%) as a light yellow solid. MS (ES, m/z): [M+1]⁺=638.3.

### Example 15

### Synthesis of (2S,4aS,14aR,14bR)-14-((S)-7,8-difluoro-6,11-dihydrodibenzo[b,e]thiepin-11-yl)-8,10-dioxo-1,3,4,5,6,8,10,14,14a,14b-decahydro-2H-2,4a-epoxypyrido[1′,2′:1,6]-[1,2,4]triazino[3,4-a]isoquinolin-9-yl 3-methoxypropanoate or (2S,4aS,14aR,14bR)-14-((R)-7,8-difluoro-6,11-dihydrodibenzo[b,e]thiepin-11-yl)-8,10-dioxo-1,3,4,5,6,8,10,14,14a,14b-decahydro-2H-2,4a-epoxypyrido[1′,2′: 1,6]-[1,2,4]triazino[3,4-a]isoquinolin-9-yl 3-methoxypropanoate

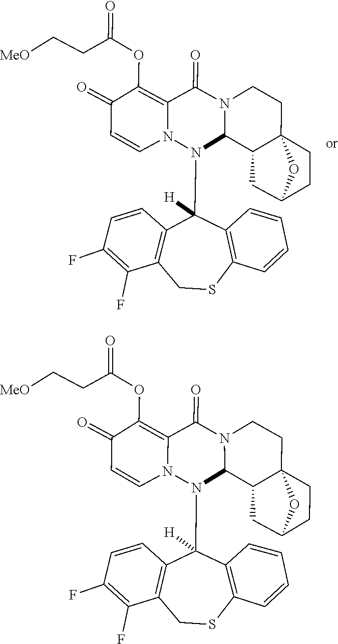

To a stirred mixture of (2S,4aS,14aR,14bR)-14-((S)-7,8-difluoro-6,11-dihydrodibenzo[b,e]-thiepin-11-yl)-9-hydroxy-1,3,4,5,6,14,14a,14b-octahydro-2H-2,4a-epoxypyrido[1′,2′:1,6]-[1,2,4]triazino[3,4-a]isoquinoline-8,10-dione or (2S,4aS,14aR,14bR)-14-((S)-7,8-difluoro-6,11-dihydrodibenzo[b,e]-thiepin-11-yl)-9-hydroxy-1,3,4,5,6,14,14a,14b-octahydro-2H-2,4a-epoxypyrido[1′,2′:1,6]-[1,2,4]triazino[3,4-a]isoquinoline-8,10-dione (20 mg, 0.036 mmol, 1.0 equiv.) and DMAP (2.22 mg, 0.018 mmol, 0.5 equiv.), and TEA (18 mg, 0.18 mmol, 5.0 equiv.) in DCM (0.5 mL) was added 3-methoxypropanoyl chloride (13 mg, 0.11 mmol, 3.0 equiv.) dropwise at 0° C. under nitrogen atmosphere and the resulting mixture was stirred for 16 h at room temperature. The crude product was purified by Prep-HPLC to afford the title compound (5 mg, 21% yield). MS (ES, m/z): [M+1]⁺=636.2.

### Example 16

### Synthesis of (((2S,4aS,14aR,14bR)-14-((S)-10-fluoro-6,11-dihydrodibenzo[b,e]thiepin-11-yl)-8,10-dioxo-1,3,4,5,6,8,10,14,14a,14b-decahydro-2H-2,4a-epoxypyrido[1′,2′: 1,6][1,2,4]triazino[3,4-a]isoquinolin-9-yl)oxy)methyl methyl carbonate

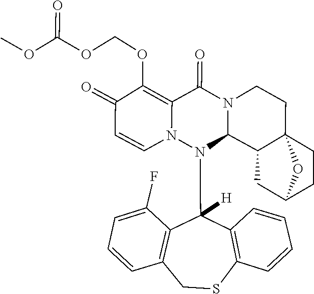 [SMILES: COC(=O)OCOc1c2n(ccc1=O)N([C@@H]1c3ccccc3SCc3cccc(F)c31)[C@@H]1[C@H]3C[C@@H]4CC[C@@]3(CCN1C2=O)O4]

To a stirred mixture of (2S,4aS,14aR,14bR)-14-((S)-10-fluoro-6,11-dihydrodibenzo-[b,e]thiepin-11-yl)-9-hydroxy-1,3,4,5,6,14,14a,14b-octahydro-2H-2,4a-epoxypyrido-[1′,2′:1,6][1,2,4]triazino[3,4-a]isoquinoline-8,10-dione (15 mg, 0.028 mmol, 1.0 equiv.), Ag₂CO₃ (23 mg, 0.085 mmol, 3 equiv.) and KI (14 mg, 0.085 mmol, 3.0 equiv.) in DMA (0.1 mL) was added chloromethyl methyl carbonate (10 mg, 0.085 mmol, 3.0 equiv.) in one portion at room temperature and the resulting mixture was stirred for 16 h at 45° C. The reaction solution was purified by Prep-HPLC to afford the title compound (1.8 mg, 10%) as a white solid. MS (ES, m/z): [M+1]⁺=620.3.

### Example 17

### Synthesis of (((2S,4aS,14aR,14bR)-14-((S)-8,9-difluoro-6,11-dihydrodibenzo[b,e]thiepin-11-yl)-8,10-dioxo-1,3,4,5,6,8,10,14,14a,14b-decahydro-2H-2,4a-epoxypyrido[1′,2′:1,6]-[1,2,4]triazino[3,4-a]isoquinolin-9-yl)oxy)methyl methyl carbonate or (((2S,4aS,14aR,14bR)-14-((R)-8,9-difluoro-6,11-dihydrodibenzo[b,e]thiepin-11-yl)-8,10-dioxo-1,3,4,5,6,8,10,14,14a,14b-decahydro-2H-2,4a-epoxypyrido[1′,2′:1,6]-[1,2,4]triazino[3,4-a]isoquinolin-9-yl)oxy)methyl methyl carbonate

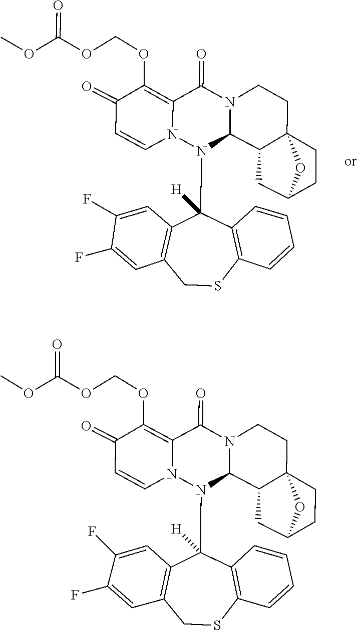 [SMILES: COC(=O)OCOc1c2n(ccc1=O)N([C@H]1c3cc(F)c(F)cc3CSc3ccccc31)[C@@H]1[C@H]3C[C@@H]4CC[C@@]3(CCN1C2=O)O4 ; COC(=O)OCOc1c2n(ccc1=O)N([C@@H]1c3cc(F)c(F)cc3CSc3ccccc31)[C@@H]1[C@H]3C[C@@H]4CC[C@@]3(CCN1C2=O)O4]

To a stirred mixture of (2S,4aS,14aR,14bR)-14-((S)-8,9-difluoro-6,11-dihydrodibenzo-[b,e]thiepin-11-yl)-9-hydroxy-1,3,4,5,6,14,14a,14b-octahydro-2H-2,4a-epoxypyrido-[1′,2′:1,6][1,2,4]triazino[3,4-a]isoquinoline-8,10-dione or (2S,4aS,14aR,14bR)-14-((R)-8,9-difluoro-6,11-dihydrodibenzo-[b,e]thiepin-11-yl)-9-hydroxy-1,3,4,5,6,14,14a,14b-octahydro-2H-2,4a-epoxypyrido-[1′,2′:1,6][1,2,4]triazino[3,4-a]isoquinoline-8,10-dione (5.0 mg, 0.01 mmol, 1.0 equiv.), Ag₂CO₃ (7.5 mg, 0.027 mmol, 3.0 equiv.) and KI (4.5 mg, 0.027 mmol, 3.0 equiv.) in DMA (0.1 mL) was added chloromethyl methyl carbonate (3.4 mg, 0.027 mmol, 3.0 equiv.) in one portion at room temperature and the resulting mixture was stirred for 16 h at 45° C. The reaction solution was purified by Prep-HPLC to afford the title compound (1.5 mg) as a white solid. MS (ES, m/z): [M+1]⁺=638.2.

### Biological Assays

### Biological Example 1

The activity of a compound against influenza viruses was assessed by the following in vitro cytopathic effect (CPE) assay.

MDCK cells were seeded into 384-well plates at a density of 2×10³ cells per well and then cultured at 37° C. and 5% CO₂ overnight. Compounds of Formula (I) were serially diluted and transferred into assay plate by Echo 555 Liquid Handler. The cells were infected with Influenza virus A/PR/8/34 (H1N1) at a concentration of 1×90% tissue culture infective doses (TCID90) per well. The final DMSO concentration was 0.5%. The cells were cultured at 37° C. and 5% CO₂ for additional 5 days. Cell viability was measured with CCK8 according to the manufacturer's instructions using microplate Spectrophotometer.

The antiviral activity (Inhibition %) of compounds was calculated using the formula below.

Inhibition (%)=(Raw datacpd−AverageVC)/(AverageCC−AverageVC)*100

The inhibitory efficacy of compounds of Formula (I) is provided in Table 3 below. ND means that EC₅₀ was not determined.

TABLE 3

| Cpd #/Structure from (Cpd. Table 1) | H₁N₁ CPE EC₅₀ (nM) |
|---|---|
| 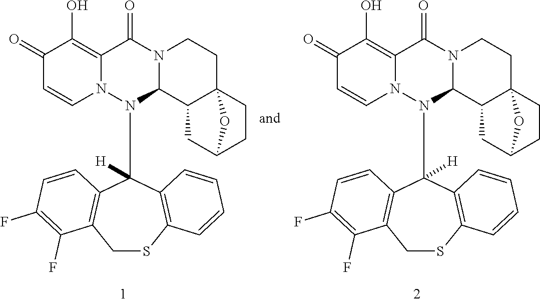 [SMILES: O=C1c2c(O)c(=O)ccn2N([C@@H]2c3ccccc3SCc3c2ccc(F)c3F)[C@@H]2[C@H]3C[C@@H]4CC[C@@]3(CCN12)O4 ; O=C1c2c(O)c(=O)ccn2N([C@H]2c3ccccc3SCc3c2ccc(F)c3F)[C@@H]2[C@H]3C[C@@H]4CC[C@@]3(CCN12)O4] | one of compounds 1 and 2 is 1.2 and the other of compounds 1 and 2 is 75 |
| 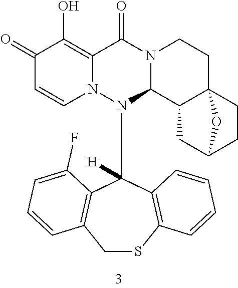 [SMILES: O=C1c2c(O)c(=O)ccn2N([C@@H]2c3ccccc3SCc3cccc(F)c32)[C@@H]2[C@H]3C[C@@H]4CC[C@@]3(CCN12)O4] | 2.1 |
| 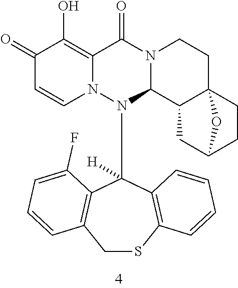 [SMILES: O=C1c2c(O)c(=O)ccn2N([C@H]2c3ccccc3SCc3cccc(F)c32)[C@@H]2[C@H]3C[C@@H]4CC[C@@]3(CCN12)O4] | 52 |
| 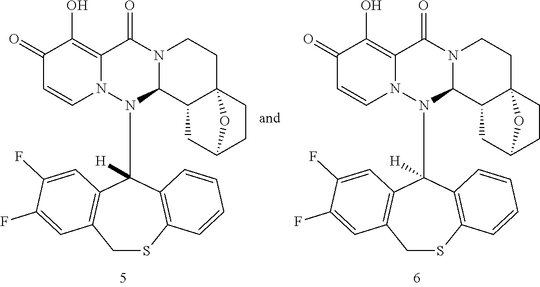 [SMILES: O=C1c2c(O)c(=O)ccn2N([C@H]2c3cc(F)c(F)cc3CSc3ccccc32)[C@@H]2[C@H]3C[C@@H]4CC[C@@]3(CCN12)O4 ; O=C1c2c(O)c(=O)ccn2N([C@@H]2c3cc(F)c(F)cc3CSc3ccccc32)[C@@H]2[C@H]3C[C@@H]4CC[C@@]3(CCN12)O4] | one of compounds 5 and 6 is 6.6 |
| 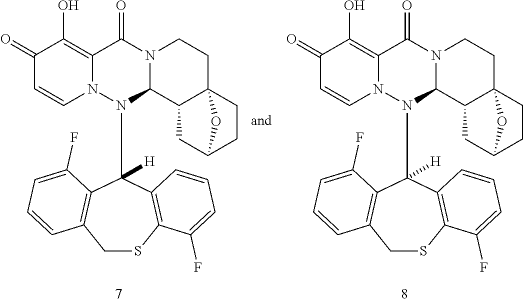 [SMILES: O=C1c2c(O)c(=O)ccn2N([C@@H]2c3cccc(F)c3SCc3cccc(F)c32)[C@@H]2[C@H]3C[C@@H]4CC[C@@]3(CCN12)O4 ; O=C1c2c(O)c(=O)ccn2N([C@H]2c3cccc(F)c3SCc3cccc(F)c32)[C@@H]2[C@H]3C[C@@H]4CC[C@@]3(CCN12)O4] | one of compounds 7 and 8 is 10.6 and the other compounds 7 and 8 is 16 |
| 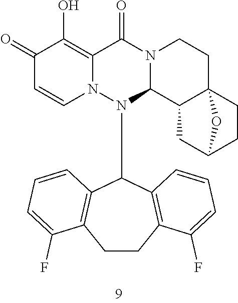 [SMILES: O=C1c2c(O)c(=O)ccn2N(C2c3cccc(F)c3CCc3c(F)cccc32)[C@@H]2[C@H]3C[C@@H]4CC[C@@]3(CCN12)O4] | 20 |
| 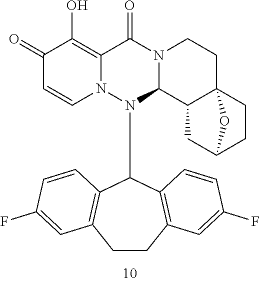 [SMILES: O=C1c2c(O)c(=O)ccn2N(C2c3ccc(F)cc3CCc3cc(F)ccc32)[C@@H]2[C@H]3C[C@@H]4CC[C@@]3(CCN12)O4] | 3.6 |
| 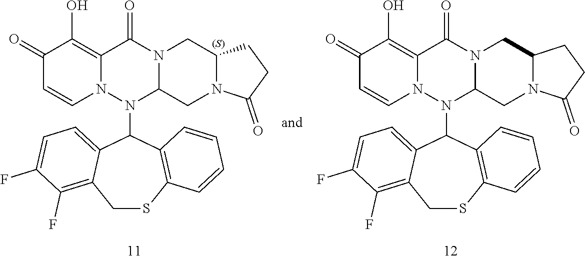 | one of compounds 11 and 12 is 53 |
| 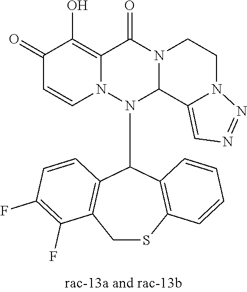 [SMILES: O=C1c2c(O)c(=O)ccn2N(C2c3ccccc3SCc3c2ccc(F)c3F)C2c3cnnn3CCN12] | one of compounds rac-13a and rac-13b is 59 |

### Biological Example 2

### Comparative PK Data for Compound 3 vs. Baloxavir Acid

Tables 4, 5, and 6 provide comparative oral exposure in mice, rat and dog of compound 3 following administration of its prodrug compound 16 vs. Baloxavir acid, depicted below:

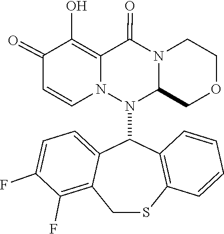 [SMILES: O=C1c2c(O)c(=O)ccn2N([C@@H]2c3ccccc3SCc3c2ccc(F)c3F)[C@@H]2COCCN12]

following administration of its prodrug Xofluza®, depicted below:

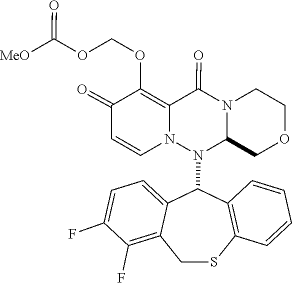 [SMILES: COC(=O)OCOc1c2n(ccc1=O)N([C@@H]1c3ccccc3SCc3c1ccc(F)c3F)[C@@H]1COCCN1C2=O]

The studies were conducted as described below.

### PK Study Protocol

Male Balb/c mice, male SD rats, male beagle dogs were used in the study. The animals were fasted for at least 12 h prior to dosing and were fed 2 h post-dose. Water was supplied ad libitum during study. In each study, three animals were included, and mice were orally dosed 10 mg/kg, rats were dosed 5 mg/kg, and dogs were dosed 2 mg/kg of compound 16 or Xofluza®, each suspended in 0.5% HPMC in water. Blood samples were taken at 0.25, 0.5, 1, 2, 4, 8 and 24 h post PO dose. The collected blood samples were then centrifuged at 3500 rpm for 10 min at 4° C. to obtain plasma. The plasma samples are transferred to polypropylene tubes and immediately stored at approximately −80° C. until analysis.

Concentration of compound 3 and Baloxavir were determined in plasma samples. Non-validated LC-MS/MS methods were used for the study.

The plasma concentration-time data from each animal was analyzed using Phoenix WinNonlin (Version 7.0, Certara). Non-compartment models were used for data analysis. PK parameters, Cₘₐₓ, AUC etc. for compound 3 in this Application and Baloxavir acid were calculated.

TABLE 4
Mice PK comparison: 10 mg/kg oral dosing.

| | Concentration of | Concentration of |
|---|---|---|
| | Compound 3 | Baloxavir |
| | following | acid following |
| | administration of | administration |
| | Compound 16 | of Xofluza ® |
| Cmax (ng/mL) | 241 | 18 |
| AUCₗₐₛₜ (ng * hr/mL) | 564 | 74 |

TABLE 5
Rat PK comparison: 5 mg/kg oral dosing.

| | Concentration of | Concentration of |
|---|---|---|
| | Compound 3 | Baloxavir |
| | following | acid following |
| | administration of | administration of |
| | Compound 16 | Xofluza ® |
| Cmax (ng/mL) | 324 | 25 |
| AUCₗₐₛₜ (ng * hr/mL) | 633 | 173 |

TABLE 6
Dog PK comparison: 2 mg/kg oral dosing.

| | Concentration of | Concentration of |
|---|---|---|
| | Compound 3 | Baloxavir |
| | following | acid following |
| | administration of | administration |
| | Compound 16 | of Xofluza ® |
| Cmax (ng/mL) | 126 | 21 |
| AUCₗₐₛₜ | 525 | 233 |
| (ng * hr/mL) | | |

The Cₘₐₓ of compound 3 is at least 6 fold higher than Baloxavir acid and AUC of compound 3 is at least 2 fold higher than Baloxavir.

### FORMULATION EXAMPLES

The following are representative pharmaceutical formulations containing a compound of the present disclosure.

### Tablet Formulation

The following ingredients are mixed intimately and pressed into single scored tablets.

| Ingredient | Quantity per tablet (mg) |
|---|---|
| compound of this disclosure | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| Ingredient | Quantity per capsule (mg) |
|---|---|
| compound of this disclosure | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal spray solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 μl of spray for each application.

While the claimed subject matter has been described in terms of various embodiments, the skilled artisan will appreciate that various modifications, substitutions, omissions, and changes may be made without departing from the spirit thereof. Accordingly, it is intended that the scope of the claimed subject matter is limited solely by the scope of the following claims, including equivalents thereof.

What is claimed:

1. A compound of Formula (IA): 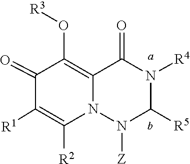
wherein:
W is hydrogen, halo, alkyl, cyano, carboxy, alkoxycarbonyl, aminocarbonyl,
alkylaminocarbonyl, dialkylaminocarbonyl, hydroxy, haloalkoxy, or alkyl substituted with one or two groups independently selected from halo, hydroxy, alkoxy, amino, alkylamino, and dialkylamino;
R² is hydrogen, halo, alkyl, haloalkoxy, hydroxy, or alkyl substituted with one, two, or three groups independently selected from halo, CN, hydroxy, alkoxy, amino, alkylamino, and dialkylamino;
R³ is hydrogen, —C(O)R⁶, —C(O)—O—R⁷, —C(R⁸R⁹)—O—C(O)R¹⁰, —C(R¹¹R¹²)—O—C(O)—OR¹³,
—P(═O)(OR¹⁴)(OR¹⁵), —(CR¹⁶R¹⁷)—O—P(═O)(OR¹⁸)(OR¹⁹), —C(O)—N(R²⁰R²¹), or —C(R²²R²¹)—O—C(O)N(R²⁴R²⁵) where R⁶, R⁷, R¹⁰, R¹³, R¹⁴, R¹⁵, R¹⁸, R¹⁹, R²⁰, R²¹, R²⁴ and R²⁵ are independently hydrogen, alkyl, phenyl, pyridyl, cycloalkyl, and a 3-6 membered heterocyclic ring wherein alkyl, phenyl, pyridyl, cycloalkyl, and a 3-6 membered heterocyclic ring are independently optionally substituted with one or two substituents independently selected from halo, cyano, hydroxy, amino, alkyl, carboxy, alkoxycarbonyl, phenyl, alkoxy, haloalkyl, and haloalkoxy;
and R⁸, R⁹, R¹¹, R¹², R¹⁶, R¹⁷, R²², and R²³ are independently hydrogen or alkyl:
R⁴ and R⁵ together with the atoms to which they are attached form a ring of formula (a), (b), (c), or (d): 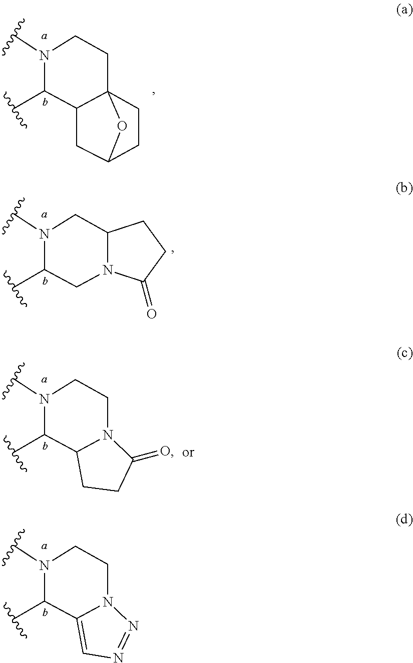 [SMILES: *C1C2CC3CCC2(CCN1*)O3 ; *C1CN2C(=O)CCC2CN1* ; *C1C2CCC(=O)N2CCN1* ; *C1c2cnnn2CCN1*]
wherein ring of formula (a), (b), (c), or (d) can optionally be substituted with one or two substituents independently selected from alkyl, alkoxy, hydroxy, halo, haloalkyl, haloalkoxy, and cyano; and
Z is:
(i) a ring of formula (i): 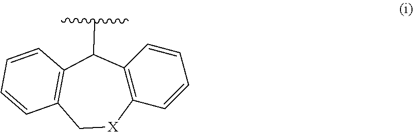
where:
wherein X is CH₂, S, S(O), S(O)₂, or O; and ring of formula (i) is substituted with one, two, three, or four substituents independently selected from hydrogen, halo, alkyl, haloalkyl, alkoxy, haloalkoxy, alkynyl, and cyano; or
(ii) a ring of formula (ii): 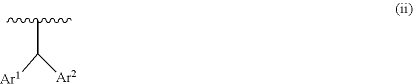
wherein Ar¹ and Ar² are independently selected from phenyl and a 5-6 membered heteroaryl ring containing 1-3 heteroatoms independently selected from N, O and S wherein each of the Ar¹ and Ar² is independently optionally substituted with one, two, or three substituents independently selected from halo, alkyl, haloalkyl, alkoxy, haloalkoxy, alkynyl, and cyan; or
a pharmaceutically acceptable salt thereof.

2. The compound of claim 1, or a pharmaceutically acceptable salt thereof, according to a structure of formula (II): 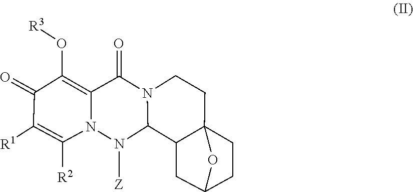
wherein:
R¹ is hydrogen, halo, alkyl, cyano, carboxy, alkoxycarbonyl, aminocarbonyl, alkylaminocarbonyl, dialkylaminocarbonyl, or alkyl substituted with one or two substituents independently selected from halo, hydroxy, alkoxy, amino, alkylamino, and dialkylamino; and
R² is hydrogen, halo, alkyl, haloalkoxy, or alkyl substituted with one, two, or three substituents independently selected from halo, CN, hydroxy, alkoxy, amino, alkylamino, and dialkylamino.

3. The compound of claim 1 or 2, or a pharmaceutically acceptable salt thereof, according to a structure of formula (IIa):

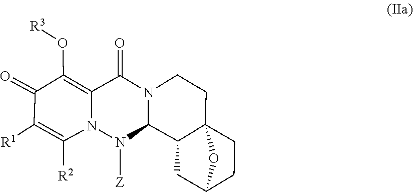

4. The compound of claim 1, or a pharmaceutically acceptable salt thereof, according to a structure of formula (III): 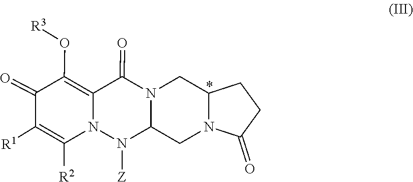
wherein:
R¹ is hydrogen, halo, alkyl, cyano, carboxy, alkoxycarbonyl, aminocarbonyl, alkylaminocarbonyl, dialkylaminocarbonyl, or alkyl substituted with one or two substituents independently selected from halo, hydroxy, alkoxy, amino, alkylamino, and dialkylamino; and
R² is hydrogen, halo, alkyl, haloalkoxy, or alkyl substituted with one, two, or substituents independently selected from halo, CN, hydroxy, alkoxy, amino, alkylamino, and dialkylamino.

5. The compound of claim 4, or a pharmaceutically acceptable salt thereof, wherein the stereochemistry at *C is (S).

6. The compound of claim 4, or a pharmaceutically acceptable salt thereof, wherein the stereochemistry at *C is (R).

7. The compound of claim 1, or a pharmaceutically acceptable salt thereof, wherein the compound of Formula (I) is according to the structure of formula (IV): 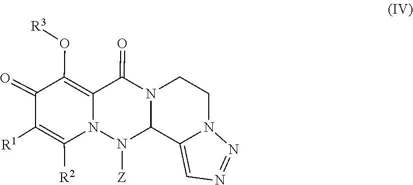
wherein:
R¹ is hydrogen, halo, alkyl, cyano, carboxy, alkoxycarbonyl, aminocarbonyl, alkylaminocarbonyl, dialkylaminocarbonyl, or alkyl substituted with one or two substituents independently selected from halo, hydroxy, alkoxy, amino, alkylamino, and dialkylamino; and
R² is hydrogen, halo, alkyl, haloalkoxy, or alkyl substituted with one, two, or three substituents independently selected from halo, CN, hydroxy, alkoxy, amino, alkylamino, and dialkylamino.

8. The compound of any one of claims 1 to 7, or a pharmaceutically acceptable salt thereof, wherein Z is a ring of formula (i).

9. The compound of any one of claims 1 to 8, or a pharmaceutically acceptable salt thereof, wherein X is S.

10. The compound of any one of claims 1 to 9, or a pharmaceutically acceptable salt thereof, wherein Z is a ring of formula:

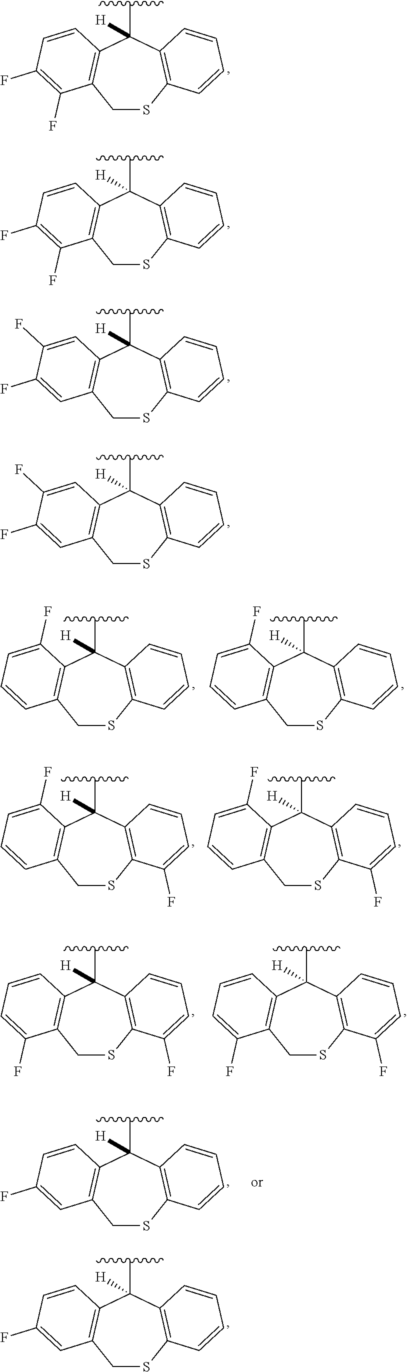

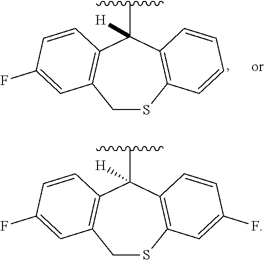

11. The compound of any one of claims 1 to 8, or a pharmaceutically acceptable salt thereof, wherein Z is a ring of formula:

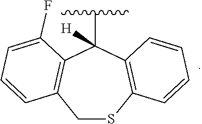 [SMILES: *[C@@H]1c2ccccc2SCc2cccc(F)c21]

12. The compound of any one of claims 2 to 11, or a pharmaceutically acceptable salt thereof, wherein R¹ and R² are independently hydrogen, methyl, fluoro, or trifluoromethyl.

13. The compound of any one of claims 1 and 8 to 11 or a pharmaceutically acceptable salt thereof, wherein R¹ and R² are independently hydrogen, methyl, fluoro, hydroxy, trifluoromethyl, or trifluoromethoxy.

14. The compound of any one of claims 1 to 11, or a pharmaceutically acceptable salt thereof, wherein R¹ and R² are each hydrogen.

15. The compound of any one of claims 1 to 14, or a pharmaceutically acceptable salt thereof, wherein R³ is hydrogen.

16. The compound of any one of claims 1 to 14, or a pharmaceutically acceptable salt thereof, wherein R³ is —C(O)R⁶ where R⁶ is alkyl optionally substituted with alkoxy.

17. The compound of claim 16, or a pharmaceutically acceptable salt thereof, wherein R⁶ is methyl, ethyl, isopropyl, -2-methylpropyl, 2-methoxyethyl, 2-methoxy-2-methylethyl, or 2-methoxy-2-methylpropyl.

18. The compound of any one of claims 1 to 14, or a pharmaceutically acceptable salt thereof, wherein R³ is —C(O)—O—R⁷ where R⁷ is alkyl.

19. The compound of claim 18, or a pharmaceutically acceptable salt thereof, wherein R⁷ is methyl, ethyl, isopropyl, 2-methylpropyl, 2-methoxyethyl, 2-methoxy-2-methylethyl, or 2-methoxy-2-methylpropyl.

20. The compound of any one of claims 1 to 14, or a pharmaceutically acceptable salt thereof, wherein R³ is —CH(R⁹)—O—C(O)R¹⁰ wherein R⁹ is hydrogen or alkyl and R¹⁰ is alkyl.

21. The compound of claim 20, or a pharmaceutically acceptable salt thereof, wherein R³ is —CH₂—OC(O)-methyl, or —CH₂—OC(O)-tert-butyl.

22. The compound of any one of claims 1 to 14, or a pharmaceutically acceptable salt thereof, wherein R³ is —CH(R¹²)—O—C(O)—OR¹³ where R¹² is hydrogen or alkyl and R¹³ is alkyl optionally substituted with alkoxy.

23. The compound of claim 22, or a pharmaceutically acceptable salt thereof, wherein R³ is —CH₂—OC(O)O-methyl, —CH(CH₃)—OC(O)O-methyl, —CH₂—OC(O)O-ethyl, —CH₂—OC(O)O-isopropyl, or —CH₂—OC(O)O-(2-methoxyethyl).

24. The compound of claim 1 selected from:
(2S,4aS,14aR,14bR)-14-((S)-7,8-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((R)-7,8-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((S)-10-fluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((R)-10-fluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((S)-8,9-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((R)-8,9-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((S)-4,10-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((R)-4,10-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-(1,9-difluoro-10,11-dihydro-5H-dibenzo[a,d][7]annulen-5-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-(2,8-difluoro-10,11-dihydro-5H-dibenzo[a,d][7]annulen-5-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(14aS)-6-(7,8-difluoro-6,11-dihydrodibenzo[b,e]thiepin-11-yl)-11-hydroxy-5a,6,14,14a-tetrahydro-1H,5H-pyrido[2,1-f]-pyrrolo[1′,2′:4,5]pyrazino[2,1-c][1,2,4]triazine-3,10,12(2H)-trione;
(14aR)-6-(7,8-difluoro-6,11-dihydrodibenzol[b,e]thiepin-11-yl)-11-hydroxy-5a,6,14,14a-tetrahydro-1H,5H-pyrido[2,1-f]-pyrrolo[1′,2′:4,5]pyrazino[2,1-c][1,2,4]triazine-3,10,12(2H)-trione;
rac-(R)-14-((S)-7,8-difluoro-6,11-dihydrodibenzo[b,e]thiepin-11-yl)-9-hydroxy-5,6,14,14a-tetrahydro-[1,2,3]triazolo-[5′,1′:3,4]pyrazino[2,1-c]pyrido[2,1-f][1,2,4]triazine-8,10-dione;
rac-(R)-14-((R)-7,8-difluoro-6,11-dihydrodibenzo[b,e]-thiepin-11-yl)-9-hydroxy-5,6,14,14a-tetrahydro-[1,2,3]triazolo-[5′,1′:3,4]pyrazino[2,1-c]pyrido[2,1-f][1,2,4]triazine-8,10-dione;
(((2S,4aS,14aR,14bR)-14-(7,8-difluoro-6,11-dihydro-dibenzo[b,e]thiepin-11-yl)-8,10-dioxo-1,3,4,5,6,8,10,14,14a,14b-decahydro-2H-2,4a-epoxypyrido-[1′,2′:1,6][1,2,4]triazino[3,4-a]isoquinolin-9-yl)-oxy)methyl methyl carbonate;
(2S,4aS,14aR,14bR)-14-(7,8-difluoro-6,11-dihydro-dibenzo[b,e]thiepin-11-yl)-8,10-dioxo-1,3,4,5,6,8,10,14,14a,14b-decahydro-2H-2,4a-epoxypyrido-[1′,2′:1,6][1,2,4]triazino[3,4-a]isoquinolin-9-yl 3-methoxypropanoate;
(((2S,4aS,14aR,14bR)-14-((S)-10-fluoro-6,11-dihydro-dibenzo[b,e]thiepin-11-yl)-8,10-dioxo-1,3,4,5,6,8,10,14,14a,14b-decahydro-2H-2,4a-epoxypyrido-[1′,2′:1,6][1,2,4]triazino[3,4-a]isoquinolin-9-yl)oxy)methyl methyl carbonate;
(((2S,4aS,14aR,14bR)-14-((S)-10-8,9-difluoro-6,11-dihydrodibenzo-[b,e]thiepin-11-yl)-8,10-dioxo-1,3,4,5,6,8,10,14,14a,14b-decahydro-2H-2,4a-epoxypyrido[1′,2′:1,6][1,2,4]triazino[3,4-a]isoquinolin-9-yl)oxy)methyl methyl carbonate;
(((2S,4aS,14aR,14bR)-14-((R)-8,9-difluoro-6,11-dihydrodibenzo-[b,e]thiepin-11-yl)-8,10-dioxo-1,3,4,5,6,8,10,14,14a, 14b-decahydro-2H-2,4a-epoxypyrido[1′,2′:1,6][1,2,4]triazino[3,4-a]isoquinolin-9-yl)oxy)methyl methyl carbonate; and
rac-(R)-14-((R)-7,8-difluoro-6,11-dihydrodibenzo[b,e]thiepin-11-yl)-9-hydroxy-5,6,14,14a-tetrahydro-[1,2,3]triazolo-[5′,1′:3,4]pyrazino[2,1-c]pyrido[2,1-f][1,2,4]triazine-8,10-dione; or
a pharmaceutically acceptable salt thereof.

25. The compound of claim 2 selected from:
(2S,4aS,14aR,14bR)-14-((S)-7,8-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((R)-7,8-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((S)-10-fluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((R)-10-fluoro-6,11-dihydro-dibenzo[b,e]thiepin-11-yl)-9-hydroxy-1,3,4,5,6,14,14a,14b-octahydro-2H-2,4a-epoxypyrido[1′,2′: 1,6][1,2,4]triazino[3,4-a]-isoquinoline-8,10-dione;
(2S,4aS,14aR,14bR)-14-((S)-8,9-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((R)-8,9-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((S)-4,10-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-((R)-4,10-difluoro-6,11-dihydro-dibenzo[b,e]thiepin-11-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-(1,9-difluoro-10,11-dihydro-5H-dibenzo[a,d][7]annulen-5-yl)-9-hydroxy-1,3,4,5,6,14,14a,14b-octahydro-2H-2,4a-epoxypyrido[1′,2′:1,6][1,2,4]triazino[3,4-a]-isoquinoline-8,10-dione;
(2S,4aS,14aR,14bR)-14-(2,8-difluoro-10,11-dihydro-5H-dibenzo[a,d][7]annulen-5-yl)-9-hydroxy-1,3,4,5,6,14,14a, 14b-octahydro-2H-2,4a-epoxypyrido[1′,2′:1,6][1,2,4]triazino[3,4-a]-isoquinoline-8,10-dione;
(((2S,4aS,14aR,14bR)-14-(7,8-difluoro-6,11-dihydro-dibenzo[b,e]thiepin-11-yl)-8,10-dioxo-1,3,4,5,6,8,10,14,14a,14b-decahydro-2H-2,4a-epoxypyrido-[1′,2′:1,6][1,2,4]triazino[3,4-a]isoquinolin-9-yl)-oxy)methyl methyl carbonate;
(2S,4aS,14aR,14bR)-14-(7,8-difluoro-6,11-dihydro-dibenzo[b,e]thiepin-11-yl)-8,10-dioxo-1,3,4,5,6,8,10,14,14a,14b-decahydro-2H-2,4a-epoxypyrido-[1′,2′:1,6][1,2,4]triazino[3,4-a]isoquinolin-9-yl 3-methoxypropanoate;
(((2S,4aS,14aR,14bR)-14-((S)-10-fluoro-6,11-dihydro-dibenzo[b,e]thiepin-11-yl)-8,10-dioxo-1,3,4,5,6,8,10,14,14a,14b-decahydro-2H-2,4a-epoxypyrido-[1′,2′:1,6][1,2,4]triazino[3,4-a]isoquinolin-9-yl)oxy)methyl methyl carbonate;
(((2S,4aS,14aR,14bR)-14-((S)-8,9-difluoro-6,11-dihydrodibenzo-[b,e]thiepin-11-yl)-8,10-dioxo-1,3,4,5,6,8,10,14,14a, 14b-decahydro-2H-2,4a-epoxypyrido[1′,2′:1,6][1,2,4]triazino[3,4-a]isoquinolin-9-yl)oxy)methyl methyl carbonate; and
(((2S,4aS,14aR,14bR)-14-((R)-8,9-difluoro-6,11-dihydrodibenzo-[b,e]thiepin-11-yl)-8,10-dioxo-1,3,4,5,6,8,10,14,14a,14b-decahydro-2H-2,4a-epoxypyrido[1′,2′:1,6][1,2,4]triazino[3,4-a]isoquinolin 9-yl)oxy)methyl methyl carbonate; or
a pharmaceutically acceptable salt thereof.

26. A pharmaceutical composition comprising a compound of any one of claims 1 to 25, or a pharmaceutically acceptable salt thereof; and a pharmaceutically acceptable excipient.

27. A method of treating a disease, caused by a virus having cap-dependent endonuclease, in a patient, comprising administering to the patient in need thereof a compound of any one of claims 1 to 25, or a pharmaceutically acceptable salt thereof, or a pharmaceutical composition of claim 26.

28. A method of treating a disease caused by Influenza A, Influenza B, and/or Influenza C viruses, in a patient, comprising administering to the patient in need thereof a compound of any one of claims 1 to 25, or a pharmaceutically acceptable salt thereof, or a pharmaceutical composition of claim 26.

29. The method of claim 28, wherein is the virus is Influenza A.

30. The method of claim 28, wherein the virus is Influenza B.

31. The method of any one of claims 27 to 30, wherein the compound of any one of claims 1 to 25, or a pharmaceutically acceptable salt thereof, or a pharmaceutical composition of claim 26 is administered with at least one additional therapeutic co-agent.

32. The method of claim 31, wherein the co-agent is an antiviral agent.

33. The method of claim 32, wherein the antiviral agent treats one or more influenza A and/or B infection(s).
